(19) United States
(12) Patent Application Publication
(10) Pub. No.: US 2025/0049776 A1
(43) Pub. Date: Feb. 13, 2025

(54) AUTOPHAGY INDUCING COMPOUNDS AND USES THEREOF, IN PARTICULAR FOR A SYSTEMIC TREATMENT OF DISEASES AND CONDITIONS

(71) Applicants: Samsara Therapeutics Inc., Lewes, DE (US); Institut National de la Santé et de la Recherche Médicale (INSERM), Paris (FR); UNIVERSITÉ DE PARIS CITÉ, Paris (FR); Sorbonne Université, Paris (FR); Assistance Publique-Hopitaux De Paris (APHP), Paris (FR)

(72) Inventors: Peter Hamley, Oxford (GB); Warren Galloway, Kronberg im Taunus (DE); Oliver Kepp, Paris (FR); Guido Kroemer, Paris (FR)

(21) Appl. No.: 18/711,529

(22) PCT Filed: Nov. 17, 2022
(86) PCT No.: PCT/EP2022/082355

§ 371 Date: May 17, 2024

(30) Foreign Application Priority Data
Nov. 17, 2021 (EP) 21208821.5

(51) Int. Cl. A61K 31/454; A61K 31/4155; A61K 31/4245; A61K 31/427; A61K 31/4439; A61K 45/06; C07D 401/14; C07D 403/04; C07D 405/14; C07D 409/04; C07D 409/14; C07D 417/14

(52) U.S. Cl. CPC A61K 31/454; A61K 31/4155; A61K 31/4245; A61K 31/427; A61K 31/4439; A61K 45/06; C07D 401/14; C07D 403/04; C07D 405/14; C07D 409/04; C07D 409/14; C07D 417/14

(57) ABSTRACT

The invention relates to pharmaceutical compositions and methods of treating autophagy related diseases and disorders, in particular for a systemic treatment thereof. The present invention relates to compounds according to Formula (I) or salts, solvates and/or hydrates thereof, wherein said compounds induce and/or stimulate the process of autophagy, as well as uses of the compounds in the treatment and prevention of autophagy related diseases and disorders. Examples are cancer, age-related diseases, and viral infection that can be effectively treated with compounds that are effective when provided systemically.

The invention relates to pharmaceutical compositions and methods of treating autophagy related diseases and disorders, in particular for a systemic treatment thereof. The present invention relates to compounds according to Formula (I) or salts, solvates and/or hydrates thereof, wherein said compounds induce and/or stimulate the process of autophagy, as well as uses of the compounds in the treatment and prevention of autophagy related diseases and disorders. Examples are cancer, age-related diseases, and viral infection that can be effectively treated with compounds that are effective when provided systemically.

### BACKGROUND OF THE INVENTION

Autophagy is the process of removing unnecessary organelles and proteins from cells that are lost or lost function, it helps maintain cell homeostasis and is a cell survival mechanism.

The cell-autonomous antimicrobial defense functions of autophagy, demonstrated initially in the case of streptococci and Mycobacterium tuberculosis have been extended to a wide variety of microbes with a caveat that most highly adapted pathogens have evolved specific protective mechanisms against autophagic elimination of microbes. Other studies have uncovered orderly intersections between autophagy and innate and adaptive immunity, T cell development, differentiation and homeostasis, and inflammatory responses. Thus, autophagy plays a large role in various diseases such as cancer, inflammatory disease, degenerative neurological disease, and immune disease.

Autophagy is a cell survival mechanism that is induced in stressed cells Towers and Thorburn (in: Therapeutic Targeting of Autophagy. EBioMedicine. 2016; 14:15-23. doi:10.1016/j.ebiom.2016.10.034) disclose that autophagy is widely accepted as cytoprotective against neurodegenerative diseases and a variety of clinical interventions are moving forward to increase autophagy as a therapeutic intervention. Autophagy has both positive and negative roles in cancer and this has led to controversy over whether or how autophagy manipulation should be attempted in cancer therapy. Nevertheless, cancer is the disease where most current activity in trying to manipulate autophagy for therapy is taking place and dozens of clinical trials are using autophagy inhibition with Chloroquine or Hydroxychloroquine in combination with other drugs for the treatment of multiple neoplasms. They review recent literature implicating autophagy in neurodegenerative diseases and cancer and highlight some of the opportunities, controversies and potential pitfalls of therapeutically targeting autophagy.

Mulcahy Levy and Thorburn (in: Autophagy in cancer: moving from understanding mechanism to improving therapy responses in patients. Cell Death Differ 27, 843-857 (2020). https://doi.org/10.1038/s41418-019-0474-7) disclose that autophagy allows for cellular material to be delivered to lysosomes for degradation resulting in basal or stress-induced turnover of cell components that provide energy and macromolecular precursors. These activities are thought to be particularly important in cancer where both tumor-promoting and tumor-inhibiting functions of autophagy have been described. Autophagy has also been intricately linked to apoptosis and programmed cell death, and understanding these interactions is becoming increasingly important in improving cancer therapy and patient outcomes. In the review, they consider how recent discoveries about how autophagy manipulation elicits its effects on cancer cell behavior can be leveraged to improve therapeutic responses.

Grainger et al. (1995, Nature Medicine 1: 1067-1073) and Reckless et al. (1997, Circulation 95: 1542-1548) have demonstrated that tamoxifen, a potent inducer of autophagy, inhibited atherosclerosis in mice models by suppressing the diet-induced formation of lipid lesions in the aorta by lowering of low-density lipoprotein (LDL) cholesterol.

CN 102516239A discloses aromatic thiazole micro-molecular organic compounds with the structural formula represented by Formula (I), or hydrates thereof or pharmaceutically acceptable salts thereof. The compounds of the invention or compositions containing the compounds can be used for sun protection, anti-ultraviolet and skin damage protection as a cosmetic additive and for inhibition of the cell apoptosis caused by excess ultraviolet irradiation and the expression of cyclooxygenase COX2.

US 2004-0116425A1 discloses related compounds useful in the treatment of diseases associated with prenylation of proteins and pharmaceutically acceptable salts thereof, to pharmaceutical compositions comprising same, and to methods for inhibiting protein prenylation in an organism using the same.

WO 2009-103432A2 extremely broadly relates to molecular probes of the formula (I) L1-R1-L-A-X as defined herein that allow for the observation of the catalytic activity of a selected caspase, cathepsin, MMP and carboxypeptidase in in vitro assays, in cells or in multicellular organisms.

In WO 2010-147653A1 compounds are extremely broadly disclosed for treating ophthalmic conditions related to mislocalization of opsin proteins, the misfolding of mutant opsin proteins and the production of toxic visual cycle products that accumulate in the eye.

WO 2017-216579A1 relates to a heterocyclic compound 1,1′-(((propane-2,2-diylbis(4,1-phenylene))bis(oxy))bis(ethane-2,1-diyl))dipyrrolidine and its medical uses, for example as an autophagy inducer.

The development of more selective autophagy inducers is needed if they are to become medicinally useful in the treatment and/or prevention of diseases where autophagy plays a role.

It is an object of the present invention to provide effective agents that can be used for the prevention and treatment of conditions and diseases that can be treated/prevented by inducing and/or stimulating of autophagy, in particular cancer, age-related diseases, and infections. Other objects and advantages will become apparent to the person of skill when studying the present description of the present invention.

In a first aspect of the present invention, the above object is solved by a compound according to Formula (I),

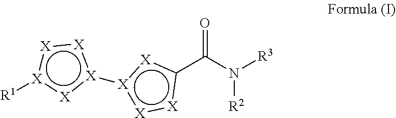

wherein
X is independently selected from chemically possible combinations of C, N, O, and S, and is optionally substituted with —CH₃, —CH₂—CH₃ or COOH,
R¹ is selected from cyclic C₅ or C₆ alkyl, optionally including chemically possible N, O, and/or S, and optionally mono- or bi-substituted with —CH₃, —NH₂, —COOH, C₁ to C₄ alkoxy, halo, trifluoromethyl, trifluoromethoxy;
R² is selected from H, CH₃, straight or branched C₂ to C₆ alkyl, optionally including chemically possible N, O, and/or S, and optionally substituted with —CH₃, —NH₂, —OH, —COOH, cyclopentyl, cyclohexyl, bicyclo[2.2.1]heptane, bicyclo[3.1.1]heptane, bicyclo[2.2.2]octane, optionally including chemically possible N, O, and/or S, and optionally substituted with —CH₃, —CH₂—CH₃, —OH, -isopropyl, —COOH, —COOCH₃, —CH₂—cyclohexyl, —NH₂,
R³ is selected from H, straight or branched C₁ to C₆ alkyl, optionally substituted with —CH₃, —OH, —CH₂—CH₃, —NH₂, —CH₂—NH₂, —CH₂—NH—CH₃, —COOH, optionally including chemically possible one or more N, O, and/or S;
C₁ to C₄ alkoxy, optionally including chemically possible one or more N, O, and/or S, and optionally mono- or bi substituted with, —NH₂, —OH, —COOH;
cyclopropyl, cyclobutyl, optionally including chemically possible one or more N, O, and/or S, and optionally mono- or bi substituted with —CH₃, —CH₂—CH₃, —NH₂, —CH₂—NH₂, —CH₂—NH—CH₃, —OH, —COOH;
with the provisio that R² and R³ are not both H or CH₃;
R² and R³ may be connected to form a 4-membered, 5-membered, 6-membered, 7-membered ring, a bicyclic ring structure of fused cyclopentyl and/or cyclohexyl, bicyclo[3.1.1]heptane, optionally including chemically possible one or more N, O, and/or S, and optionally being mono- or bi-substituted with straight or branched C₁ to C₆ alkyl, optionally substituted with —CH₃, —OH, —CH₂—CH₃, —NH₂, —NH—CH₃, —CH₂—NH₂, —CH₂—NH—CH₃, —OH, —COOH, —COOCH₃, ═O, isopropyl, sulfonamide, and optionally including chemically possible one or more N, O, and/or S;
with the provisio that R², R³, or the combination thereof comprises at least one NH or NH₂; a physiologically acceptable salt, a solvate, a hydrate, an enantiomer or a polymorph thereof.

The compounds described herein were shown surprisingly to be highly effective autophagy inducers/stimulators. The present inventors thus invented compounds for the induction and/or stimulation of autophagy in disease- and undesired conditions-related cells of a patient or subject. In the case of a compound represented by the general Formula according to the present invention, the autophagy is effectively induced and/or stimulated. Furthermore, the compounds exhibit higher systemic (peripheral) concentrations, compared to the CNS or brain, preferably a minimum of a 2:1 peripheral/brain ratio, after an administration thereof, which makes them ideal candidates for the induction and/or stimulation of autophagy in disease- and undesired conditions-related cells of a patient or subject that can be treated via a systemic administration.

It was surprisingly found that the compounds according to Formula I, as an autophagy inducer, were effective and exhibits many advantages compared to other autophagy-related treatment options. In comparison with prior art compounds as tested (including data not shown), an improvement of the present invention lies in the unexpected observation that the compounds described herein are highly effective autophagy inducers (see Examples below). Based on these results this is evidence that these compounds are active in autophagy-related diseases as disclosed herein, preferably autophagy-related diseases and conditions that can be treated via a systemic administration. Furthermore, examples for this activity are shown in the Examples below which also relate to specific activities in models of viral infections and diseases involving protein misfolding. By way of an exemplar medical application, the compounds according to Formula 170/171

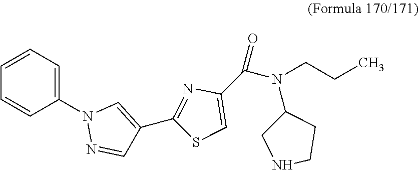 [SMILES: CCCN(C(=O)c1csc(-c2cnn(-c3ccccc3)c2)n1)C1CCNC1]

were shown to be surprisingly efficacious (see Examples below).

Preferred is a compound according to the present invention according to the following formula II,

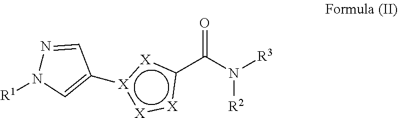

wherein X, R¹, R² and R³ are as above, a physiologically acceptable salt, a solvate, a hydrate, an enantiomer or a polymorph thereof.

Preferred is a compound according to the present invention according to the following formula III,

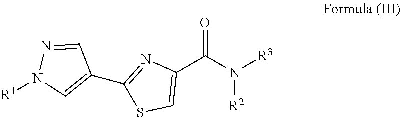

wherein R¹, R² and R³ are as above, a physiologically acceptable salt, a solvate, a hydrate, an enantiomer or a polymorph thereof.
   Preferred is a compound according to the present invention, wherein R¹ is selected from

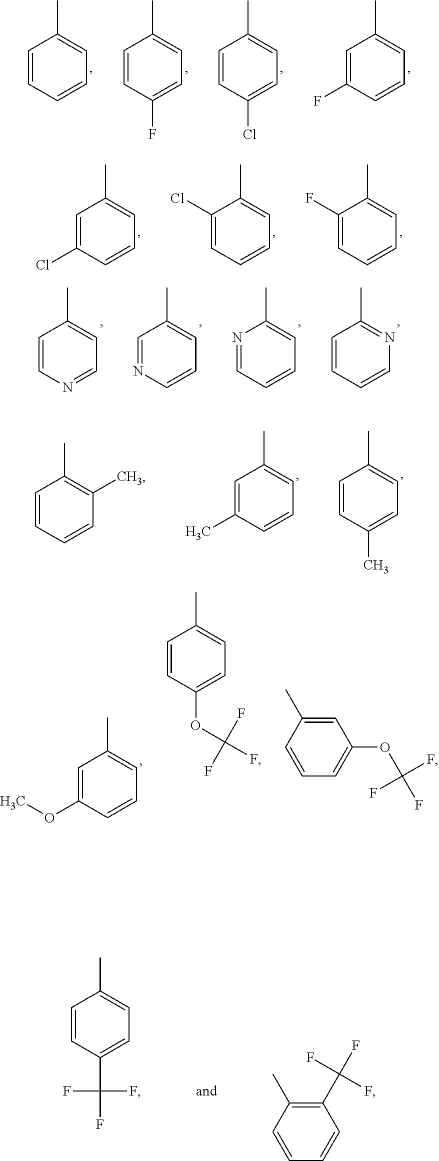

a physiologically acceptable salt, a solvate, a hydrate, an enantiomer or a polymorph thereof. Preferred is a compound according to the present invention, wherein R¹ and X are as above, R² is selected from H, —CH₃, —CH₂—CH₃, —CH₂—CH₂—CH₃, —CH₂—CH₂—NH₂, —CH₂—CH₂—CH₂—CH₂—NH₂, —CH₂—CH₂—CH₂—CH₂—CH₂—NH₂, —CH₂—CH₂—O—CH₂—CH₂—NH₂, cyclohexyl, —CH₂(CH₃)—CH₂—NH₂

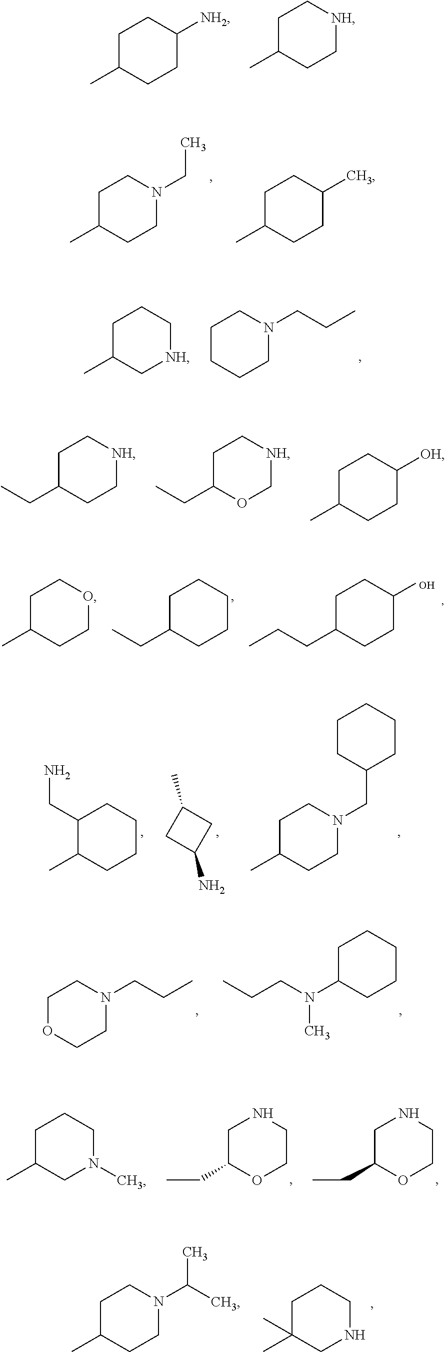

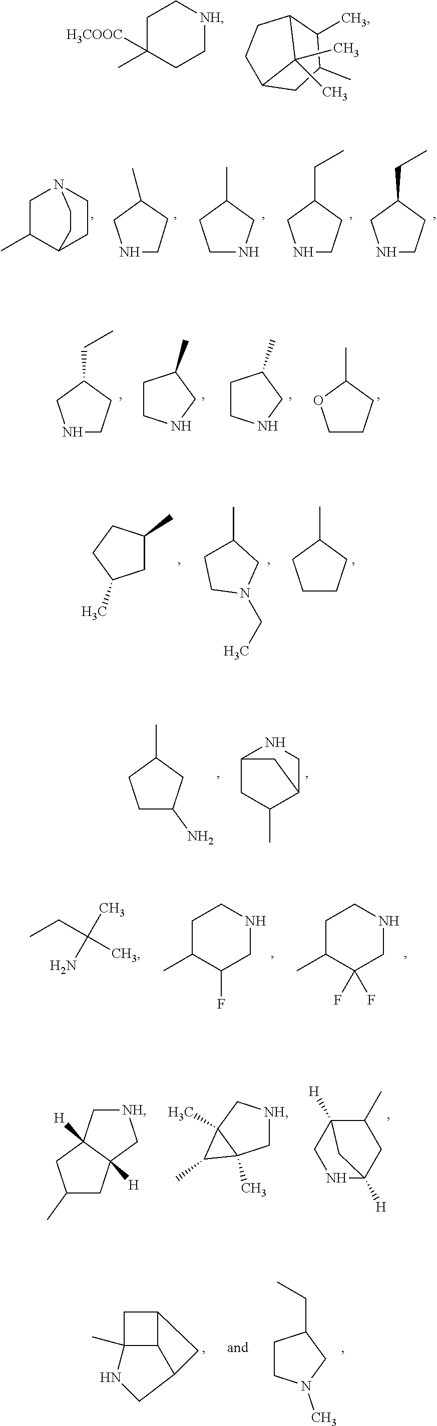

R³ is selected from H, —CH₃, —CH₂—CH₃, propyl, isopropyl, —CH₂—CH₂—OH, —CH₂—CH₂—NH₂, —CH₂—CH₂—CH₂—NH₂, —CH(NH₂)—CH₃, —CH₂—CH₂—CH₂—CH₂—CH₂—NH₂, —CH₂—CH₂—O—CH₂—CH₂—NH₂, —CH₂—CH(CH₃)₂, —CH(CH₃)₂, —CH₂—CH(NH₂)—CH₃, —CH(—CH₃)—CH₂—NH₂, —CH₂— CH₂—N(CH₃)₂,

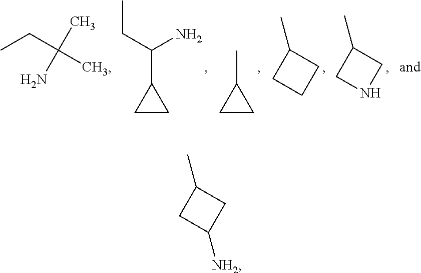

and R² and R³ may be connected to form a ring selected from

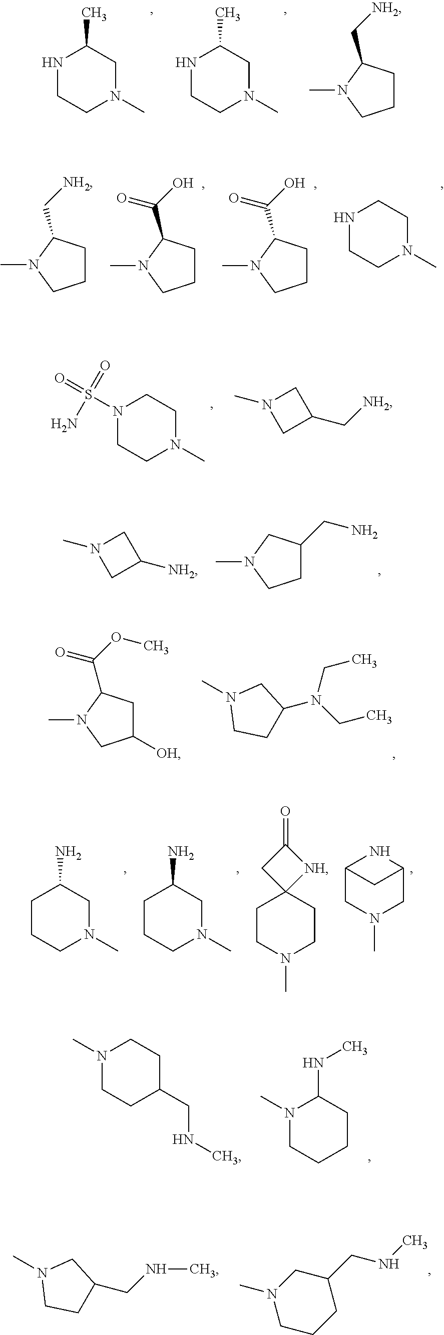

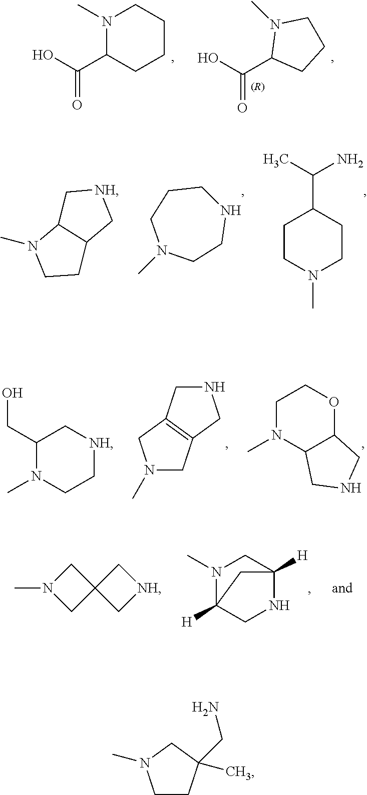

a physiologically acceptable salt, a solvate, a hydrate, an enantiomer or a polymorph thereof.
Preferred is a compound according to the present invention according to the following formula IV,

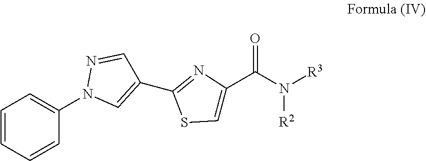

wherein R² is selected from

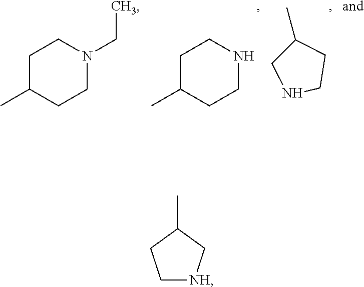

R³ is selected from H, CH₃, —CH₂—CH₂—CH₃, and isopropyl, or
R² and R³ form a ring selected from

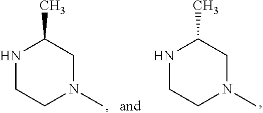

a physiologically acceptable salt, a solvate, a hydrate, an enantiomer or a polymorph thereof.

Preferred is a compound according to the present invention selected from the following:
5-(1-phenyl-1H-pyrazol-4-yl)-N-propyl-N-[(3S)-pyrrolidin-3-yl]-1H-pyrrole-2-carboxamide (Formula 93);
5-(1-phenyl-1H-pyrazol-4-yl)-N-propyl-N-[(3R)-pyrrolidin-3-yl]-1H-pyrrole-2-carboxamide (Formula 94);
2-(5-phenylthiophen-2-yl)-N-propyl-N-(pyrrolidin-3-yl)-1,3-thiazole-4-carboxamide (Formula 122);
4-(1-phenyl-1H-pyrazol-4-yl)-N-propyl-N-(pyrrolidin-3-yl)-1H-pyrrole-2-carboxamide (Formula 123);
2-(1-phenyl-1H-pyrazol-4-yl)-N-(piperidin-4-yl)-N-(propan-2-yl)-1,3-thiazole-4-carboxamide (Formula 163);
2-(1-phenyl-1H-pyrazol-4-yl)-N-propyl-N-[(3S)-pyrrolidin-3-yl]-1,3-thiazole-4-carboxamide (Formula 170);
2-(1-phenyl-1H-pyrazol-4-yl)-N-propyl-N-[(3R)-pyrrolidin-3-yl]-1,3-thiazole-4-carboxamide (Formula 171); and
   a physiologically acceptable salt, a solvate, a hydrate, an enantiomer or a polymorph thereof.

Further preferred compounds according to the present invention are disclosed in tables 2, 2a, and 2b, as below.

The term “pharmaceutically acceptable salt” refers to a pharmaceutically acceptable organic or inorganic salt of the compound of the invention. This may include addition salts of inorganic acids such as hydrochloride, hydrobromide, hydroiodide, sulphate, phosphate, diphosphate and nitrate or of organic acids such as acetate, maleate, fumarate, tartrate, succinate, citrate, lactate, methanesulphonate, p-toluenesulphonate, palmoate and stearate. Exemplary salts also include oxalate, chloride, bromide, iodide, bisulphate, acid phosphate, isonicotinate, salicylate, acid citrate, oleate, tannate, pantothenate, bitartrate, ascorbate, gentisinate, gluconate, glucuronate, saccharate, formate, benzoate, glutamate, ethanesulfonate, and benzenesulfonate salts. For other examples of pharmaceutically acceptable salts, reference can be made to Gould (1986, Int J Pharm 33: 201-217).

According to a further aspect of the invention, there is a provided a method for producing a compound according to the present invention, comprising the steps according to any one of general experimental procedures as disclosed herein, preferably methods 1 to 4.

According to a further aspect of the invention, there is a provided a pharmaceutical composition comprising a pharmaceutically effective amount of the compound according to the present invention, and a pharmaceutically or therapeutically acceptable excipient or carrier.

The term “pharmaceutically or therapeutically acceptable excipient or carrier” refers to a solid or liquid filler, diluent or encapsulating substance which does not interfere with the effectiveness or the biological activity of the active ingredients and which is not toxic to the host, which may be either humans or animals, to which it is administered. Depending upon the particular route of administration, a variety of pharmaceutically-acceptable carriers such as those well known in the art may be used. Non-limiting examples include sugars, starches, cellulose and its derivatives, malt, gelatin, talc, calcium sulfate, vegetable oils, synthetic oils, polyols, alginic acid, phosphate buffered solutions, emulsifiers, isotonic saline, and pyrogen-free water. Pharmaceutically acceptable carriers or excipients also include diluents (fillers, bulking agents, e.g. lactose, microcrystalline cellulose), disintegrants (e.g. sodium starch glycolate, croscarmellose sodium), binders (e.g. PVP, HPMC), lubricants (e.g. magnesium stearate), glidants (e.g. colloidal SiO₂), solvents/co-solvents (e.g. aqueous vehicle, Propylene glycol, glycerol), buffering agents (e.g. citrate, gluconates, lactates), preservatives (e.g. Na benzoate, parabens (Me, Pr and Bu), BKC), anti-oxidants (e.g. BHT, BHA, Ascorbic acid), wetting agents (e.g. polysorbates, sorbitan esters), thickening agents (e.g. methylcellulose or hydroxyethylcellulose), sweetening agents (e.g. sorbitol, saccharin, aspartame, acesulfame), flavoring agents (e.g. peppermint, lemon oils, butterscotch, etc.), humectants (e.g. propylene, glycol, glycerol, sorbitol). Other suitable pharmaceutically acceptable excipients are inter alia described in Remington's Pharmaceutical Sciences, 15^{th} Ed., Mack Publishing Co., New Jersey (1991) and Bauer et al., Pharmazeutische Technologic, 5^{th} Ed., Govi-Verlag Frankfurt (1997). The person skilled in the art knows suitable formulations for the compounds according to the present invention, and will readily be able to choose suitable pharmaceutically acceptable carriers or excipients, depending, e.g., on the formulation and administration route of the pharmaceutical composition.

All suitable modes of administration are contemplated according to the invention. For example, administration of the medicament may be via oral, subcutaneous, direct intravenous, slow intravenous infusion, continuous intravenous infusion, intravenous or epidural patient controlled analgesia (PCA and PCEA), intramuscular, intrathecal, epidural, intracistemal, intraperitoneal, transdermal, topical, buccal, sublingual, transmucosal, inhalation, intra-atricular, intranasal, rectal or ocular routes, abuse deterrent and abuse resistant formulations, sterile solutions suspensions and depots for parenteral use, and the like, administered as immediate release, sustained release, delayed release, controlled release, extended release and the like. The medicament may be formulated in discrete dosage units and can be prepared by any of the methods well known in the art of pharmacy. The pharmaceutical composition of the present invention can be formulated using methods known in the art to provide rapid, sustained or delayed release of the active ingredient after administration to a mammal. In general, at least one compound according to the present invention is admixed with at least one pharmaceutically acceptable carrier and/or excipient.

In addition to the aforementioned compounds of the invention, the pharmaceutical composition can contain two or more compounds according to the present invention and also other therapeutically active substances.

The dosage of the pharmaceutical composition according to the present invention can be appropriately selected according to the route of administration, the subject to be administered, the target disease and its severity, age, sex weight, individual differences and disease state. Dosage may be repeated several times a day.

According to the present invention, a mammalian subject can be preferably selected from a mouse, rat, cat, dog, rabbit, goat, sheep, horse, camel, lama, cow, monkey, a farm animal, a sport animal, and a pet, and a human.

Further provided is a compound of the invention as defined herein for use in the prevention and/or treatment of conditions and/or diseases in a mammalian subject, such as a human. A condition and/or disease suitable for treatment according to the relevant aspects of the invention is one which is characterized by defective or insufficient autophagy or which would benefit from modulation such as induction of autophagy. The compounds exhibit higher systemic (peripheral) concentrations, compared to the CNS or brain, preferably a minimum of a 2:1 peripheral/brain ratio, after an administration thereof, which makes them ideal candidates for the induction and/or stimulation of autophagy in disease- and undesired conditions-related cells of a patient or subject that can be treated via a systemic administration.

The invention further encompasses the use of a compound of the invention as an autophagy inducer. The use may be a cosmetic use and/or in vitro, for example in an in vitro assay.

U.S. Pat. No. 9,138,400, for example, relates to a cosmetic process for detoxifying the skin and/or for combating cutaneous aging, comprising the topical application on the skin of a composition that comprises at least one activator of the autophagy of cells of the skin. Eckhart L, Tschachler E, and Gruber F (in: Autophagic Control of Skin Aging. Front Cell Dev Biol. 2019; 7:143. Published 2019 Jul. 30. doi:10.3389/fcell.2019.00143) review the evidence for cell type-specific roles of autophagy in the skin and their differential contributions to aging.

Autophagy inhibition plays a key role in the pathogenesis of inherited autophagic vacuolar myopathies (including Danon disease, X-linked myopathy with excessive autophagy, and infantile autophagic vacuolar myopathy), all of which are characterized by lysosomal defects and an accumulation of autophagic vacuoles. Autophagic vacuolar myopathies and cardiomyopathies can also be secondary to treatment with autophagy-inhibiting drugs (chloroquine, hydroxychloroquine and colchicine), which are used experimentally to interrogate autophagic flux and clinically to treat malaria, rheumatological diseases, and gout.

Autophagy impairment has also been implicated in the pathogenesis of inclusion body myositis, an age-associated inflammatory myopathy that is currently refractory to any form of treatment, along with other muscular dystrophies such as tibial muscular dystrophy.

Modified basal autophagy levels are seen in rheumatoid arthritis and osteoarthritis. Other aspects of the immune response associated with dysfunctional autophagy are seen in neutrophils from patients with familial Mediterranean fever and in monocytes from patients with TNF receptor-associated periodic syndrome, both of which are autoinflammatory disorders. Moreover, autophagy regulates an important neutrophil function, the generation of neutrophil extracellular traps (NETs). The important role of autophagy in the induction of NET formation has been studied in several neutrophil—associated disorders such as gout, sepsis, and lung fibrosis. Furthermore, there is a relationship between autophagy and the secretory pathway in mammalian macrophages for the release of IL1B, demonstrating a possible alternative role of autophagy for protein trafficking. This role has also been implied in neutrophils through exposure of protein epitopes on NETs by acidified LC3-positive vacuoles in sepsis and anti-neutrophil cytoplasmic antibody associated vasculitis. Patients with chronic kidney disease also have impaired autophagy activation in leukocytes, which is closely related to their cardiac abnormalities. There is also evidence for altered autophagy in pancreatic beta cells and in adipocytes of patients with type 2 diabetes.

A crucial role for therapy-induced autophagy in cancer cells has recently emerged, in modulating the interface of cancer cells and the immune system; primarily, by affecting the nature of danger signaling (i.e., the signaling cascade that facilitates the exposure and/or release of danger signals) associated with immunogenic cell death (ICD).

Therefore, compounds according to the present invention are for use in the prevention and/or treatment of an autophagy-related disease or condition in a mammalian subject, such as a human.

By “treatment” or “treating” is meant any treatment of a disease or disorder, in a mammal, including: preventing or protecting against the disease or disorder, that is, causing, the clinical symptoms of the disease not to develop; inhibiting the disease, that is, arresting or suppressing the development of clinical symptoms; and/or relieving the disease, that is, causing the regression of clinical symptoms. By “amelioration” is meant the prevention, reduction or palliation of a state, or improvement of the state of a subject; the amelioration of a stress is the counteracting of the negative aspects of a stress. Amelioration includes, but does not require complete recovery or complete prevention of a stress.

Preferred is a compound for use according to the present invention, wherein said autophagy-related disease or condition is selected from the group consisting of systemic lupus erythematosus, cancer, liver diseases, al antitrypsin deficiency, Charcot Marie Tooth syndrome, Rett Syndrome, Sickle Cell disease, Wilson Disease, amyloidosis, Gaucher's diseases, lysosomal and glycogen storage disorders, cystic fibrosis; viral infection and diseases, human cytomegalovirus (HCMV) infection, hepatitis B, human immunodeficiency virus infection, Zika virus infection, coronavirus infection, HCoV-229E, HCoV-NL63, betacoronavirus infection, such as HCoV—OC43, SARS-CoV-1, HCoV—HKU1, MERS-CoV or SARS-CoV-2, bacterial infections, metabolic disorders, diabetes, fibrosis, wound healing disorders, Niemann-Pick type C (NPC) disease, fibrinogen storage disease (FSB), inclusion body disease (IBD), muscular dystrophy, Duchenne muscular dystrophy, Limb-girdle muscular dystrophy, myopathy, myofibrillar myopathy, hereditary myopathy, diabetic cardiomyopathy, anti-inflammatory disorders, autoimmune diseases, multiple sclerosis, rheumatoid arthritis, irritable bowel syndrome, Crohn's disease, vascular disorders, coronary artery diseases, myocardial infarction, unstable angina pectoris, atherosclerosis or vasculitis, Behcet's syndrome, giant cell arteritis, polymyalgia rheumatica, Wegener's granulomatosis, Churg-Strauss syndrome, vasculitis, Henoch-Schonlein purpura, Kawasaki disease, viral infection or replication, pox virus infection, herpes virus infection, asthma, allergic rhinitis, COPD, osteoporosis, organ transplant rejection, psoriasis, hypertrophic scarring (keloid formation), adhesion formations following general or gynecological surgery, lung fibrosis, liver fibrosis, kidney fibrosis, disorders caused by intracellular parasites, malaria, tuberculosis, neuropathic pain, post-operative phantom limb pain or postherpetic neuralgia, allergies, antigen induced recall response, immune response suppression, muscle degeneration and atrophy, frailty in aging, spinal cord injury, and diseases and conditions involving misfolded and/or nonfolded proteins that can be effectively treated with compounds that are effective when provided systemically as well as age-related forms of the diseases and conditions (such as cancer, and metabolic syndrome). Particularly preferred is Charcot Marie Tooth syndrome.

Cheon S Y, Kim H, Rubinsztein D C, and Lee J E. (in: Autophagy, Cellular Aging and Age-related Human Diseases. Exp Neurobiol 2019; 28:643-657. https://doi.org/10.5607/en.2019.28.6.643) disclose that during aging, cellular factors suggested as the cause of aging have been reported to be associated with progressively compromised autophagy. Dysfunctional autophagy may contribute to age-related diseases, such as neurodegenerative disease, cancer, and metabolic syndrome, in the elderly. Therefore, restoration of impaired autophagy to normal may help to prevent age-related disease and extend lifespan and longevity. They provide an overview of the mechanisms of autophagy underlying cellular aging and the consequent disease.

Similarly, Leidal, A. M., Levine, B. & Debnath, J. (in: Autophagy and the cell biology of age-related disease. Nat Cell Biol 20, 1338-1348 (2018). https://doi.org/10.1038/s41556-018-0235-8) review that autophagy declines with age and that impaired autophagy predisposes individuals to age-related diseases, whereas interventions that stimulate autophagy often promote longevity.

Also, Vaccaro Maria Ines, De Tata Vincenzo, and Gonzalez Claudio Daniel (in: Editorial: Autophagy in Endocrine-Metabolic Diseases Associated With Aging; Frontiers in Endocrinology, 11, 2020, pp 572, 0 10.3389/fendo.2020.00572) present a special issue containing a collection of 12 articles covering a broad range of key topics on the interplay of the different types of autophagy alterations with aging, endocrine-metabolic, and degenerative diseases.

Further preferred is the compound for use according to the present invention, wherein said prevention and/or treatment comprises a combination of at least two compounds for use according to the present invention, and/or a combination with at least one additional pharmaceutically active substance for said autophagy-related disease or condition.

Further preferred is the compound for use according to the present invention, wherein said prevention and/or treatment comprises a systemic treatment, exhibiting higher systemic (peripheral) concentrations compared to the CNS or brain.

It is to be understood that the present compound and/or a pharmaceutical composition comprising the present compound is for use to be administered to a human patient. The term “administering” means administration of a sole therapeutic agent or in combination with another therapeutic agent. It is thus envisaged that the pharmaceutical composition of the present invention is employed in co-therapy approaches, i.e. in co-administration with other medicaments or drugs and/or any other therapeutic agent which might be beneficial in the context of the methods of the present invention. Nevertheless, the other medicaments or drugs and/or any other therapeutic agent can be administered separately from the compound for use, if required, as long as they act in combination (i.e. directly and/or indirectly, preferably synergistically) with the present compound(s) (for use).

In another aspect of the compound for use according to the present invention the prevention and/or treatment further comprises detecting and/or monitoring in said subject a response of at least one autophagy-biomarker. The biomarker is preferably selected from the group consisting of BECN1, ATG8/LC3 family, including LC3A, LC3B, LC3C), LC3-II, ULK1, p62, NBR1, ATG5 and ATG7.

This monitoring usually is performed on a biologically sample taken from the mammalian subject, and comprises commonly known tests, for example antibody based, PCR based, and the like. The tests are repeated over time, and can be compared to control samples and/or samples taken earlier from the mammalian subject. The results help the attending physician to maintain or modify the course of a treatment, usually based on the severity of the clinical symptoms of the autophagy-related disease and/or condition as treated.

In another aspect thereof, the present invention provides methods for preventing and/or treating an autophagy-related disease and/or condition in a mammalian subject, such as a human, comprising administering to said mammal an effective amount of a compound or a pharmaceutical composition according to the present invention. preferably, said prevention and/or treatment comprises a systemic treatment, exhibiting higher systemic (peripheral) concentrations compared to the CNS or brain.

Preferred is the method according to the present invention, wherein said autophagy-related disease or condition is selected from the group consisting of systemic lupus erythematosus, cancer, liver diseases, al antitrypsin deficiency, Charcot Marie Tooth syndrome, Rett Syndrome, Sickle Cell disease, Wilson Disease, amyloidosis, Gaucher's diseases, lysosomal and glycogen storage disorders, cystic fibrosis; viral infection and diseases, human cytomegalovirus (HCMV) infection, hepatitis B, human immunodeficiency virus infection, Zika virus infection, coronavirus infection, HCoV-229E, HCoV-NL63, betacoronavirus infection, such as HCoV—OC43, SARS-CoV-1, HCoV—HKU1, MERS-CoV or SARS-CoV-2, bacterial infections, metabolic disorders, diabetes, fibrosis, wound healing disorders, Niemann-Pick type C (NPC) disease, fibrinogen storage disease (FSB), inclusion body disease (IBD), muscular dystrophy, Duchenne muscular dystrophy, Limb-girdle muscular dystrophy, myopathy, myofibrillar myopathy, hereditary myopathy, diabetic cardiomyopathy, anti-inflammatory disorders, autoimmune diseases, multiple sclerosis, rheumatoid arthritis, irritable bowel syndrome, Crohn's disease, vascular disorders, coronary artery diseases, myocardial infarction, unstable angina pectoris, atherosclerosis or vasculitis, Behcet's syndrome, giant cell arteritis, polymyalgia rheumatica, Wegener's granulomatosis, Churg-Strauss syndrome, vasculitis, Henoch-Schonlein purpura, Kawasaki disease, viral infection or replication, pox virus infection, herpes virus infection, asthma, allergic rhinitis, COPD, osteoporosis, organ transplant rejection, psoriasis, hypertrophic scarring (keloid formation), adhesion formations following general or gynecological surgery, lung fibrosis, liver fibrosis, kidney fibrosis, disorders caused by intracellular parasites, malaria, tuberculosis, neuropathic pain, post-operative phantom limb pain or postherpetic neuralgia, allergies, antigen induced recall response, immune response suppression, muscle degeneration and atrophy, frailty in aging, spinal cord injury, and diseases and conditions involving misfolded and/or nonfolded proteins that can be effectively treated with compounds that are effective when provided systemically. Particularly preferred is Charcot Marie Tooth syndrome.

The dosage of the pharmaceutical composition to be administered according to the present invention can be appropriately selected according to the route of administration, the subject to be administered, the target disease and its severity, age, sex weight, individual differences and disease state. Dosage may be repeated several times a day.

According to the present invention, a mammalian subject can be preferably selected from a mouse, rat, cat, dog, rabbit, goat, sheep, horse, camel, lama, cow, monkey, a farm animal, a sport animal, and a pet, and a human.

In addition to the aforementioned compounds of the invention, the pharmaceutical composition as administered can contain two or more compounds according to the present invention and also other therapeutically active substances. In the method, the compound for use can be provided and/or is administered as a suitable pharmaceutical composition as discussed above. The compounds can be administered alone or in combination with other active compounds—for example with medicaments already known for the treatment of the aforementioned conditions and/or diseases, whereby in the latter case a favorable additive, amplifying or preferably synergistically effect is noticed.

In another aspect of the method according to the present invention the prevention and/or treatment further comprises detecting and/or monitoring in said subject a response of at least one autophagy-biomarker. The biomarker is preferably selected from the group of BECN1, and the ATG8/LC3 family, including LC3A, LC3B, LC3C), LC3-II, ULK1, p62, NBR1, ATG5 and ATG7.

This monitoring usually is performed on a biologically sample taken from the mammalian subject, and comprises commonly known tests, for example antibody based, PCR based, and the like. The tests are repeated over time, and can be compared to control samples and/or samples taken earlier from the mammalian subject. The results help the attending physician to maintain or modify the course of a treatment, usually based on the severity of the clinical symptoms of the autophagy-related disease and/or condition as treated.

It was surprisingly found that the compounds according to Formula I, as an autophagy inducer, were effective and exhibits many advantages compared to other autophagy-related treatment options, particularly as a combination treatment. In comparison with prior art compounds as tested (including data not shown), an improvement of the present invention lies in the unexpected observation that the compounds described herein are highly effective autophagy inducers (see Examples below). By way of an exemplar medical application, the compounds according to Formula 170/171

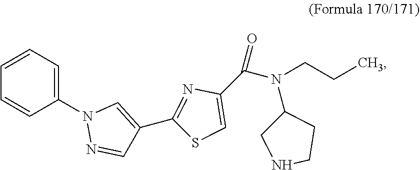 [SMILES: CCCN(C(=O)c1csc(-c2cnn(-c3ccccc3)c2)n1)C1CCNC1]

were shown to be surprisingly efficacious (see Examples below).

The present invention will now be described further in the following examples, nevertheless, without being limited thereto. For the purposes of the present invention, all references as cited herein are incorporated by reference in their entireties.

### EXAMPLES

The following examples have been performed in part using preferred compounds as disclosed. Nevertheless, it should be understood that the present invention is not in any way limited thereto, and the person of skill is readily able to adjust the conditions as described to other compounds according to the present invention.

Compounds of the present invention were synthesized from commercially available starting materials, in preferably by following the relevant general experimental procedures as given below (general experimental procedures 1-4).

### Abbreviations Used:

Boc tertiary-butoxycarbonyl
tBu tertiary butyl
DCM dichloromethane
DIPEA N,N-Diisopropylethylamine
DMF dimethylformamide
DMSO dimethylsulfoxide
dppf 1, 1′-bis(diphenylphosphino)ferrocene
eq equivalents
ESI electrospray ionisation
Hz hertz
H hours
HATU 1-[Bis(dimethylamino)methylene]-1H-1,2,3-triazolo[4,5-b]pyridinium 3-oxide
hexafluorophosphate
HPLC high performance liquid chromatography
J coupling constant in Hz
LCMS liquid chromatography-mass spectrometry
min minutes
NMR nuclear magnetic resonance
N normal (equivalents per litre)
Ph phenyl
RT retention time
rt room temperature
SFC supercritical fluid chromatography
THE tetrahydrofuran
TLC thin-layer chromatography
vol volumes
X-Phos 2-Dicyclohexylphosphin-2′,4′,6′-triisopropylbiphenyl
X-Phos Pd G2 Second generation XPhos Precatalyst (X-Phos aminobiphenyl palladium chloride precatalyst); Chloro(2-dicyclohexylphosphino-2′,4′,6′-triisopropyl-1,1′-biphenyl)[2-(2′-amino-1,1′-biphenyl)]palladium(II)

### General Experimental Procedure 1

### Schematic Overview Over Synthesis:

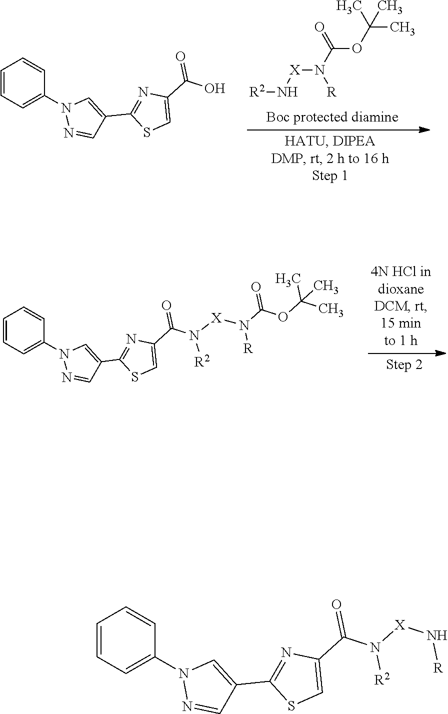

### Typical General Conditions

### Step 1—Synthesis of Boc-Protected Intermediate

To a solution of 2-(1-phenyl-1H-pyrazol-4-yl) thiazole-4-carboxylic acid (CAS. No. 137576-90-0, 1.0 eq) in DMF (10 vol) at rt, HATU (1.5 eq) and DIPEA (3.0 eq) were added. The reaction mixture was stirred for 15-20 min, then the Boc-protected amine derivative (1.0 eq) was added to reaction mixture at rt. The reaction mixture was stirred at rt for 2 h to 16 h (the progress of the reaction was monitored by TLC and LCMS analysis). After completion of the reaction, the reaction mixture was poured into cold water (10 vol) and extracted with ethyl acetate (3×5 vol). The combined organic fractions were washed with cold water 3-4 times, dried over sodium sulfate and concentrated under reduced pressure. The crude material was purified by flash column chromatography (CombiFlash® system) to provide the Boc-protected intermediate.

### Step 2—Deprotection

To a stirred solution of the Boc-protected intermediate (1.0 eq) in DCM (10 vol) at 0° C. or rt, 4N HCl in dioxane was added. The resulting reaction mixture was stirred at rt for 15 min to 1 h (the progress of the reaction was monitored by TLC and LCMS analysis). After completion of reaction, the reaction mixture was concentrated under reduced pressure and triturated (typically with diethyl ether or DCM) to provide the target compound.

### General Experimental Procedure 2

### Schematic Overview of Synthesis (X=Unspecified Heteroatom; R¹=Aryl or Heteroaryl [Unsubstituted and Substituted]):

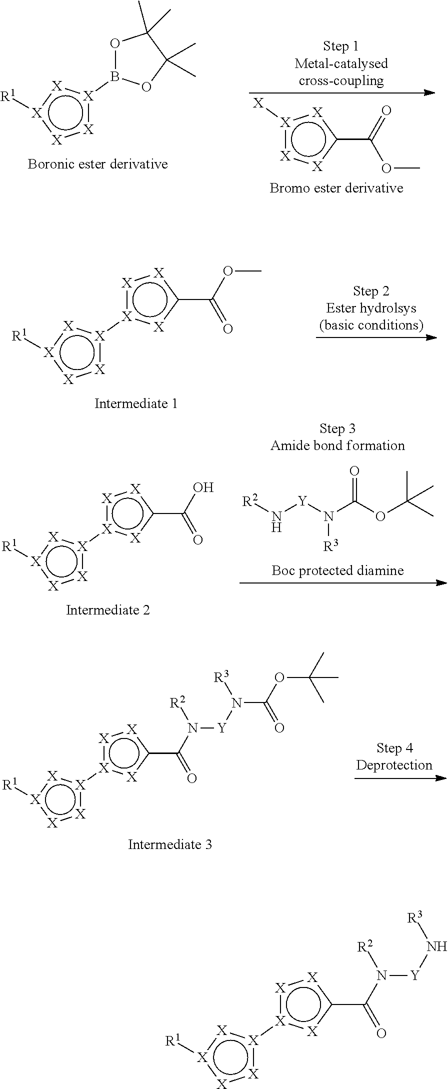

### Typical General Conditions

### Step 1—Cross-Coupling

To a stirred solution of the Boronic ester derivative (1.0 eq) and the Bromo ester derivative (1.2 eq) in a suitable solvent (DMF or 1,4-dioxane:water (9:1, 10 vol) at rt, a suitable base (potassium phosphate or potassium carbonate or sodium carbonate; 2.0 to 3.0 eq) was added. The reaction mixture was degassed with nitrogen gas for 10 min. A suitable palladium catalyst (Pd(PPh₃)₄ or PdCl₂(dppf).DCM or X-Phos Pd G2 or (t-Bu₃P)₂Pd; 0.1 eq) was added. The reaction mixture was again degassed with nitrogen gas for 10 min and then heated at 80° C.-100° C. for 3 h to 16 h (the progress of the reaction was monitored by TLC and LCMS analysis). After completion of the reaction, the reaction mixture was diluted with water (10 vol) and extracted with ethyl acetate (3×5 vol). The combined organic fractions were dried over anhydrous sodium sulfate and concentrated under reduced pressure. The crude material was purified by flash column chromatography (CombiFlash® system) to provide Intermediate 1.

### Step 2—Ester Hydrolysis

To a stirred solution of Intermediate 1 (1.0 eq) in a suitable solvent (typically THF (10 vol) or MeOH (10 vol) or EtOH (10 vol)) at rt, a suitable base (typically LiOH (8.0 eq) in water (10 vol) or NaOH (5.0 eq) in water (10 vol)) was added. The reaction mixture was stirred at rt for 2 h to 16 h (the progress of the reaction was monitored by TLC and LCMS analysis). After completion of reaction, the reaction mixture was distilled out and extracted with ethyl acetate (2×5 vol). The aqueous layer was acidified (typically with 1N HCl or a saturated solution of citric acid) and extracted with ethyl acetate (3×10 vol). The combined organic fractions were dried over sodium sulfate and concentrated under reduced pressure to provide Intermediate 2.

### Step 3—Formation of Boc-Protected Amide

To a stirred solution of Intermediate 2 in DMF (10 vol) at room temperature, HATU (1.5 eq) and DIPEA (3.0 eq) were added. The reaction mixture was stirred at rt for 15-30 min, then the Boc-protected amine (1.0 eq) was added into reaction mixture. The reaction mixture was stirred at rt (the progress of the reaction was monitored by TLC and LCMS analysis). After completion of reaction (typically ˜4h), the reaction mixture was poured into cold water (10 vol) and extracted with ethyl acetate (3×10 vol). The combined organic fractions were washed with cold water 3-4 times, dried over sodium sulfate and concentrated under reduced pressure. The crude material was purified by flash column chromatography (CombiFlash® system) to provide Intermediate 3.

### Step 4—Deprotection

To a stirred solution of Intermediate 3 (1.0 eq) in DCM (10 vol) at 0° C., 4N HCl in Dioxane (5 vol) was added. The reaction mixture was stirred at rt for 1 h to 4 h (the progress of the reaction was monitored by TLC and LCMS analysis). After completion of reaction, the reaction mixture was concentrated under reduced pressure to provide crude material, which was triturated with diethyl ether to provide the target compound.

### General Experimental Procedure 3

### Schematic Overview of Synthesis (R¹=Aryl or Heteroaryl [Unsubstituted and Substituted] or

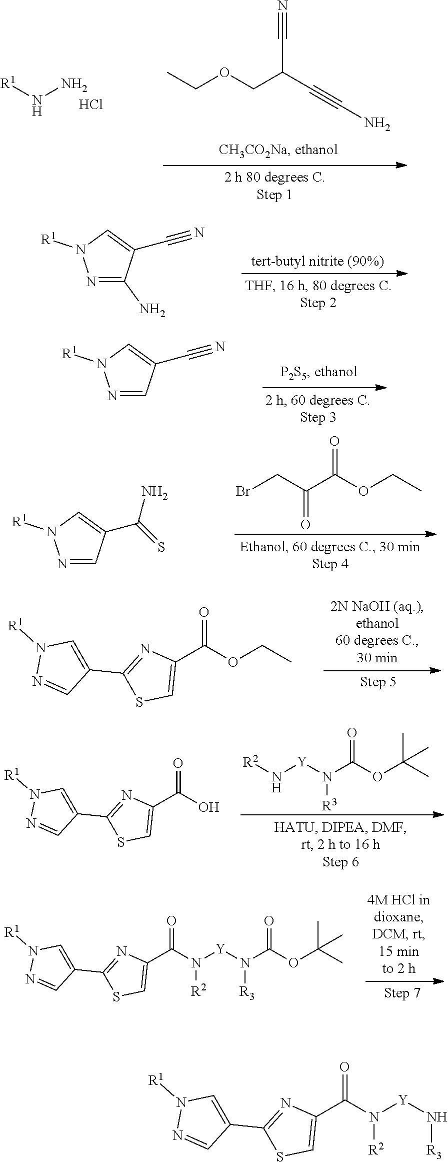

### Typical General Conditions

### Step-1: Synthesis of 5-Amino-1H-Pyrazole-4-Carbonitrile Intermediate

To a stirred solution of the appropriate hydrazine hydrochloride (20.69 mmol) in ethanol (50 mL) at rt, sodium acetate (3.39 g, 41.39 mmol) and 2-(ethoxymethylene)malononitrile (2.52 g, 20.69 mmol) were added. The reaction mixture was refluxed at 80° C. for 2h (the progress of reaction was monitored by TLC and LCMS analysis). After completion of the reaction, the reaction mixture was concentrated under reduced pressure. The residual material was washed with cold water (50 mL) and extracted with ethyl acetate (3×100 mL). The combined organic fractions were dried over anhydrous sodium sulfate and concentrated under reduced pressure to provide the crude 5-amino-1-aryl-1H-pyrazole-4-carbonitrile.

### Step-2: Synthesis of 1H-Pyrazole-4-Carbonitrile Intermediate:

To a stirred solution of the crude 5-amino-1-aryl-1H-pyrazole-4-carbonitrile (18.24 mmol) in THF (50 mL) at rt, tert-butyl nitrite(90%)(4.13 g,36.49 mmol) was added. The reaction mixture was refluxed at 80° C. for 16 h (the progress of the reaction was monitored by TLC and LCMS analysis). After completion of reaction, the reaction mixture was concentrated under reduced pressure. The residual material was purified by flash column chromatography to provide the 1H-pyrazole-4-carbonitrile.

### Step-3: Synthesis of 1H-Pyrazole-4-Carbothioamide Intermediate

To a stirred solution of the 1H-pyrazole-4-carbonitrile (15.5 mmol) in ethanol (37 mL) at rt, phosphorus pentasulfide (6.93 g, 31.1 mmol) was added. The reaction mixture was refluxed at 60° C. for 2 h (the progress of the reaction was monitored by TLC and LCMS analysis). After completion of the reaction, the reaction mixture was distilled under vacuum. The crude reaction mixture was washed with sodium bicarbonate solution (100 mL) until neutralization of pH and extracted with ethyl acetate (3×150 mL). The combined organic fractions were dried over sodium sulfate, concentrated under reduced pressure and triturated with diethyl ether to afford the crude 1H-pyrazole-4-carbothioamide.

### Step—4: Synthesis of Ethyl 2-Aryl-1H-Pyrazol-4-Yl)Thiazole-4-Carboxylate

To a stirred solution of the 1H-pyrazole-4-carbothioamide (14.70 mmol) in ethanol (50 mL) at rt, ethyl 3-bromo-2-oxopropanoate (3.14 g, 16.17 mmol) was added. The reaction mixture was refluxed at 60° C. for 30 min (the progress of the reaction was monitored by TLC and LCMS analysis). After completion of the reaction, the reaction mixture was distilled under vacuum to provide the ethyl 2-aryl-1H-pyrazol-4-yl)thiazole-4-carboxylate.

### Step—5: Synthesis of 2-Aryl-1H-Pyrazol-4-Yl)Thiazole-4-Carboxylic Acid

To a stirred solution of the ethyl 2-aryl-1H-pyrazol-4-yl)thiazole-4-carboxylate (14.4 mmol) in ethanol (60 mL) at rt, 2 N NaOH solution (21.62 mL) was added. The reaction mixture was heated at 60° C. for 30 minutes (the progress of the reaction was monitored by TLC and LCMS analysis). After completion of the reaction, the reaction mixture was poured into water (50 mL) and extracted with ethyl acetate (3×50 mL). The aqueous layer was separated out and acidified with 2 M HCl solution to generated a precipitate, which was isolated by filtration and dried under reduced pressure to provide the 2-aryl-1H-pyrazol-4-yl)thiazole-4-carboxylic acid.

### Step—6: Synthesis of Final Thiazole-4-Carboxamide Compound or N-Boc Protected Thiazole-4-Carboxamide Intermediate

To a stirred solution of 2-aryl-1H-pyrazol-4-yl)thiazole-4-carboxylic acid (0.58 mmol) in DMF (4 mL) at rt, HATU (0.33 g, 0.87 mmol) and DIPEA (0.21 g, 1.7 mmol) were added. The reaction mixture was stirred for 30 min and then the required amine (0.58 mmol) was added at rt. The reaction mixture was stirred at rt for 4h (the progress of the reaction was monitored by TLC and LCMS analysis). After completion of the reaction, the reaction mixture was poured into cold water (15 mL) and extracted with ethyl acetate (3×50 mL). The combined organic fractions were washed with cold water 3-4 times, dried over sodium sulfate and concentrated under reduced pressure. The crude material was purified by flash column chromatography) to provide the product as a solid.

### Step−7: Deprotection of Thiazole-4-Carboxamide to Obtain Final Product

To a stirred solution of the N-Boc protected intermediate from Step 6 above (0.35 mmol) in DCM (2 mL, 10V) at 0° C., 4M HCl in Dioxane (1 mL, 5V) was added. The reaction mixture was stirred at room temperature for 1h (the progress of the reaction was monitored by TLC and LCMS analysis). After completion of reaction, the reaction mixture was concentrated under reduced pressure, and the crude material triturated with diethyl ether to provide the target thiazole-4-carboxamide hydrochloride as a solid.

### General Experimental Procedure 4

### Schematic Overview of Synthesis

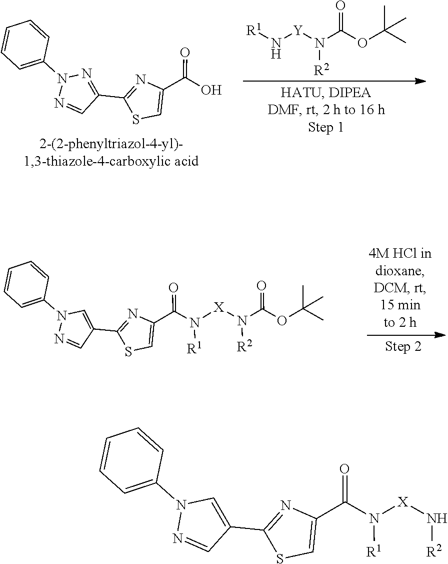

### General Experimental Procedure 5

### Schematic Overview of Synthesis (R¹=Aryl or Heteroaryl [Unsubstituted and Substituted]):

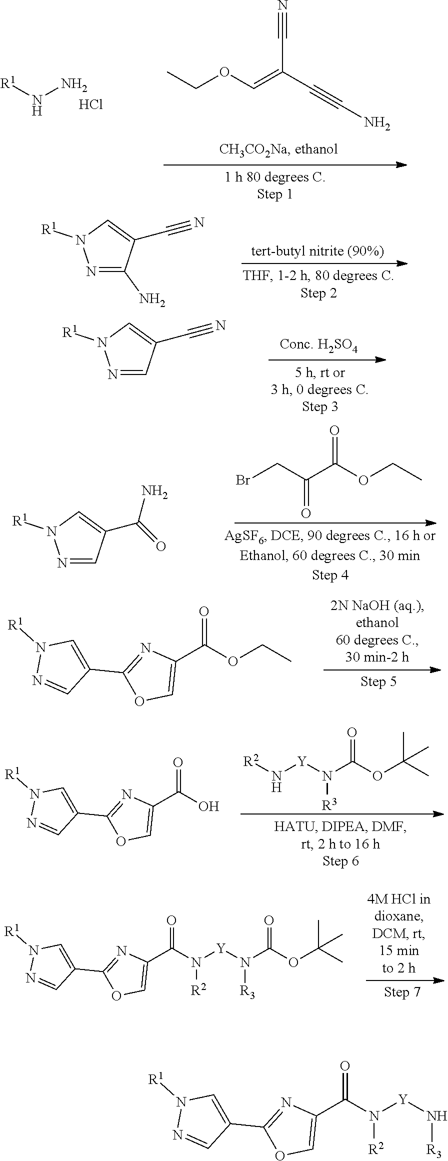

### General Experimental Procedure 6

### Schematic Overview of Synthesis (R¹=Alkyl):

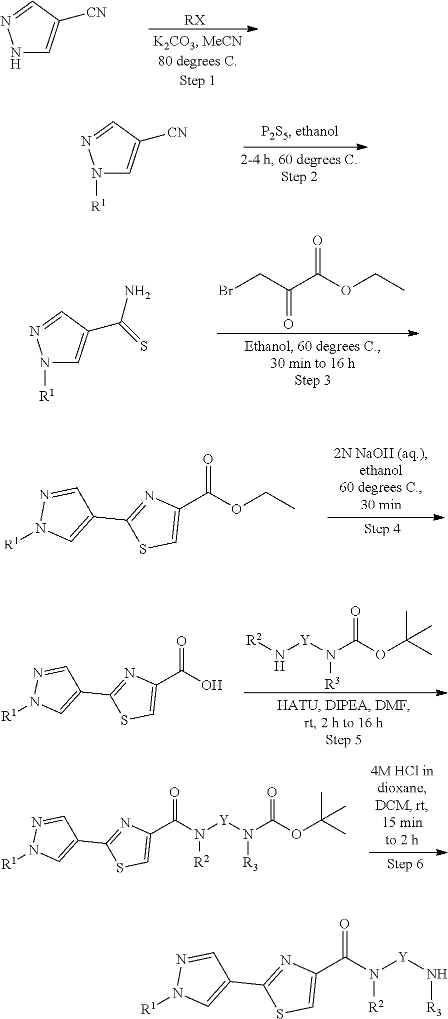

Overview of synthetic route towards Compound 337

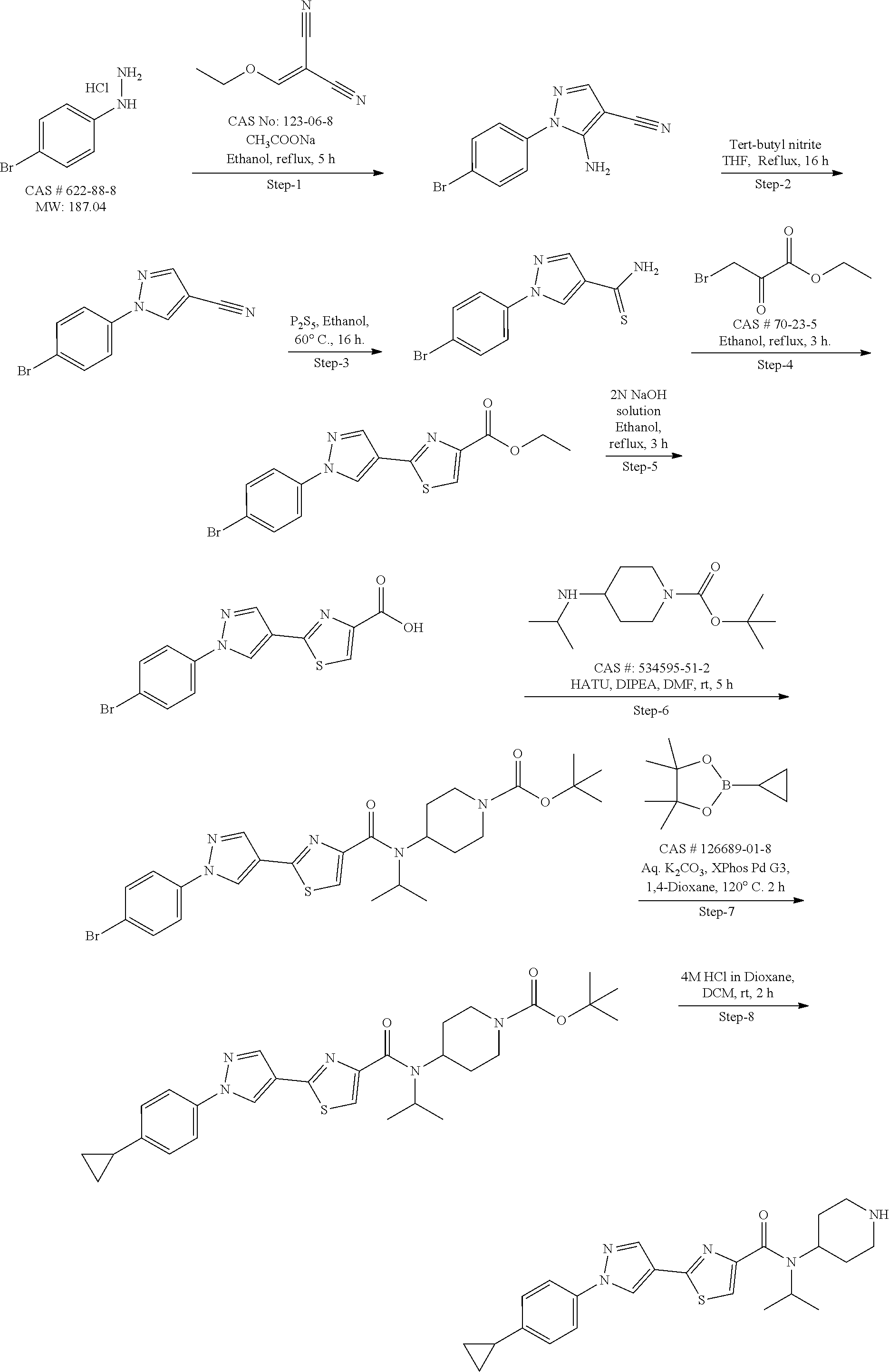

Overview of synthetic route towards Compound 342

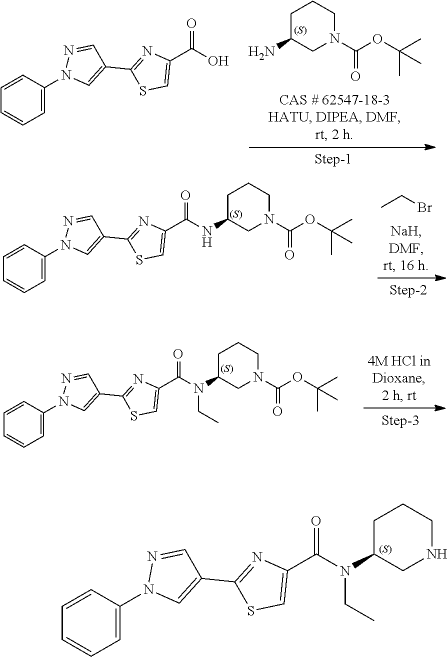

### Overview of Synthetic Route Towards Compound 343

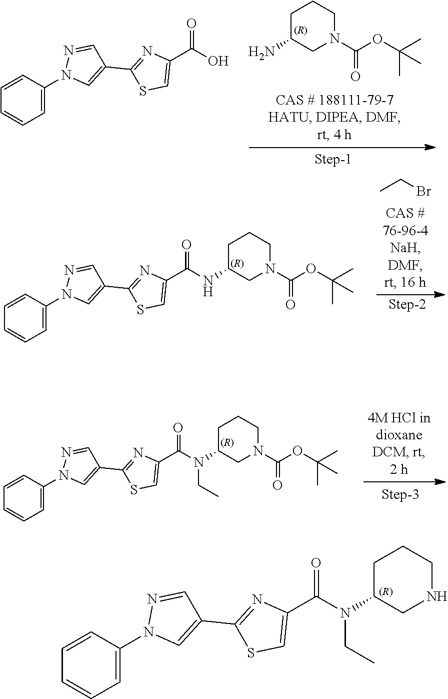

### Full Synthetic Details for a Representative Set of Compounds

### 1. Synthesis of N-isopropyl-2-(1-phenyl-1H-pyrazol-4-yl)-N-(piperidin-4-yl)thiazole-4-carboxamide hydrochloride (Compound 163)

### Overall Synthetic Scheme

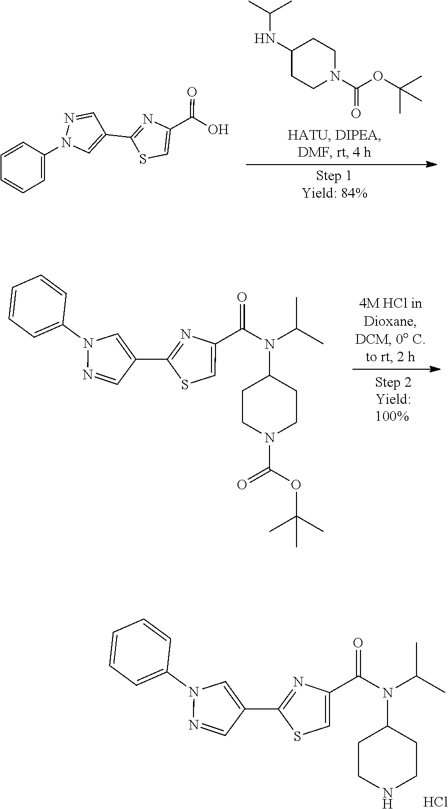 [SMILES: O=C(O)c1csc(-c2cnn(-c3ccccc3)c2)n1 ; CC(C)NC1CCN(C(=O)OC(C)(C)C)CC1 ; CC(C)N(C(=O)c1csc(-c2cnn(-c3ccccc3)c2)n1)C1CCN(C(=O)OC(C)(C)C)CC1 ; CC(C)N(C(=O)c1csc(-c2cnn(-c3ccccc3)c2)n1)C1CCNCC1 ; Cl]

### Synthetic Procedure—Prepared According to General Experimental Procedure 2.

In step 1 2-(1-phenyl-1H-pyrazol-4-yl)thiazole-4-carboxylic acid (1.5 g, 5.52 mmol) and 4-(isopropylamino)piperidine-1-carboxylate (1.6 g, 6.63 mmol) were used. The solution of 2-(1-phenyl-1H-pyrazol-4-yl)thiazole-4-carboxylic acid (1.5 g, 5.52 mmol), HATU (3.1 g, 8.29 mmol) and DIPEA (2.1 g, 16.58 mmol) in in DMF (15 mL) was stirred at rt for 15 min prior to the addition of 4-(isopropylamino)piperidine-1-carboxylate (1.6 g, 6.63 mmol). The reaction mixture was stirred at rt for 4 h. The crude material was purified by flash column chromatography (CombiFlash® system; 40-45% ethyl acetate/hexane) to provide tert-butyl 4-(N-isopropyl-2-(1-phenyl-1H-pyrazol-4-yl)thiazole-4-carboxamido)piperidine-1-carboxylate (2.3 g, 84% yield) as an off-white solid.

LCMS [ESI, M+1]: 440.1[M−56] (RT: 2.400 min, Purity: 95.09%),

¹H NMR (400 MHz, d₆-DMSO): δ 9.18 (s, 1H), 8.25 (s, 1H), 7.95 (d, J=7.8 Hz, 2H), 7.84 (d, J=9.3 Hz, 1H), 7.55 (t, J=7.9 Hz, 2H), 7.38 (t, J=7.4 Hz, 1H), 4.08-3.95 (m, 1H), 3.87 (s, 2H), 2.78 (d, J=25.5 Hz, 2H), 2.72-2.64 (m, 2H), 1.89-1.58 (m, 2H), 1.48 (d, J=14.9 Hz, 1H), 1.39 (d, J=13.9 Hz, 9H), 1.30-1.10 (m, 6H).

In step 2, 4—(N-isopropyl-2-(1-phenyl-1H-pyrazol-4-yl)thiazole-4-carboxamido)piperidine-1-carboxylate (2.3 g, 4.64 mmol) in DCM (23 mL) was used. The solution was cooled to 0° C. prior to the addition of 4M HCl in Dioxane (11.5 mL, 5V). The reaction mixture was stirred for 2h. The crude material was triturated with diethyl ether to provide N-isopropyl-2-(1-phenyl-1H-pyrazol-4-yl)-N-(piperidin-4-yl)thiazole-4-carboxamide hydrochloride (Compound 163: 2.0 g, 100% yield) as a white solid.

LCMS [ESI, M+1]: 396.2 (RT: 1.399 min, Purity: 95.09%),

HPLC Purity: RT: 4.799 min, Purity: 95.39%,

¹H NMR (400 MHz, D₂O): δ 8.55 (s, 1H), 8.09 (s, 1H), 7.58 (d, J=7.1 Hz, 3H), 7.45 (t, J=7.7 Hz, 2H), 7.34 (t, J=7.5 Hz, 1H), 3.97 (m, 1H), 3.57 (m, 1H), 3.47 (d, J=11.5 Hz, 2H), 3.05 (d, J=11.9 Hz, 1H), 2.87 (d, J=10.9 Hz, 2H), 1.92 (d, J=12.5 Hz, 2H), 1.41 (s, 1H), 1.16 (d, J=6.2 Hz, 6H).

### 2. Synthesis of (R)-5-(1-phenyl-1H-pyrazol-4-yl)-N-propyl-N-(pyrrolidin-3-yl)-1H-pyrrole-2-carboxamide hydrochloride (Compound 94)

### Overall Synthetic Scheme

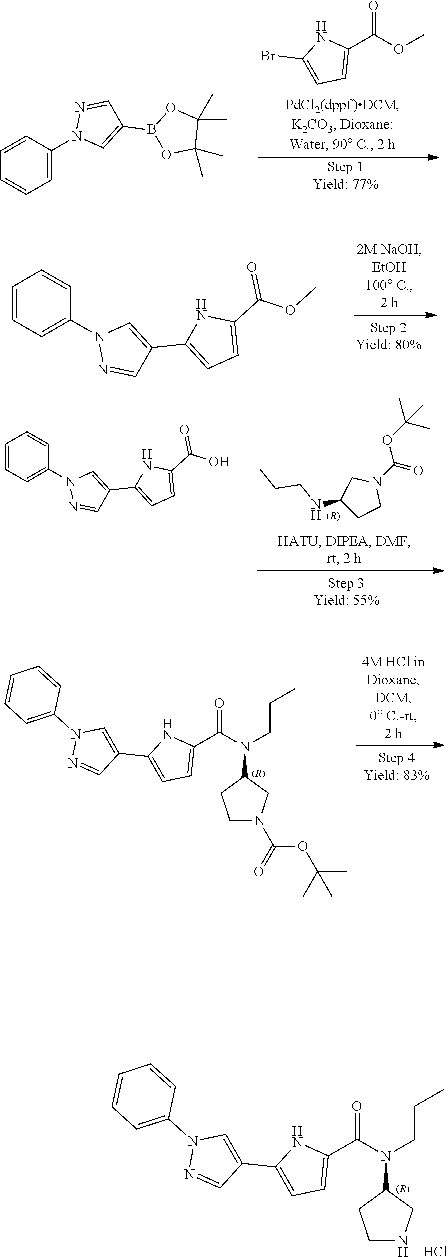

### Synthetic Procedure—Prepared by General Experimental Procedure 3.

In step 1, 1-phenyl-4-(4,4,5,5-tetramethyl-1,3,2-dioxaborolan-2-yl)-1H-pyrazole (3.1 g, 11.81 mmol) and K₂CO₃ (3.39 g, 24.5 mmol) in water (20 mL) were added to a stirred solution of methyl 5-bromo-1H-pyrrole-2-carboxylate (2.0 g, 9.80 mmol) in dioxane (15 mL) at room temperature. PdCl₂(dppf)DCM complex (0.8 g, 0.98 mmol) was used as the palladium catalyst. The reaction mixture was stirred at 90° C. for 2 h. The crude material was purified by flash column chromatography (CombiFlash® system; 20-30% ethyl acetate/hexane] to provide methyl 5-(1-phenyl-1H-pyrazol-4-yl)-1H-pyrrole-2-carboxylate (2.0 g, 77%) as a beige solid. LCMS [ESI, M+1]: 267.9 (RT: 1.896 min, Purity: 83.25%),

In step 2 2M NaOH (20 mL) was added to a stirred solution of methyl 5-(1-phenyl-1H-pyrazol-4-yl)-1H-pyrrole-2-carboxylate (2.0 g, 7.48 mmol) in EtOH (20 mL) at room temperature. The reaction mixture was heated at 100° C. for 2 h. The following purification procedure was used: after completion of reaction, the reaction mixture was concentrated under reduced pressure. The residue was dissolved in water (50 mL) and extracted with diethyl ether (3×100 mL). The aqueous layer was slowly acidified with a saturated solution of citric acid up to pH ˜4-5. A solid was precipitate out, which was isolated by filtration and dried under reduced pressure to provide 5-(1-phenyl-1H-pyrazol-4-yl)-1H-pyrrole-2-carboxylic acid (1.5 g, 80%) as an off-white solid.

LCMS [ESI, M−18]: 254.2 (RT: 1.641 min, Purity: 88.07%),

HPLC Purity: RT: 6.453 min, Purity: 88.13%

¹H NMR (400 MHz, d₆-DMSO): δ 12.25 (s, 1H), 11.93 (d, J=8.0 Hz, 1H), 8.95 (s, 1H), 8.25 (s, 1H), 7.80 (d, J=7.7 Hz, 2H), 7.54 (t, J=8.0 Hz, 2H), 7.34 (t, J=7.4 Hz, 1H), 6.79 (dd, J=3.6, 2.4 Hz, 1H), 6.46 (dd, J=3.6, 2.5 Hz, 1H).

In step 3 the mixture of 5-(1-phenyl-1H-pyrazol-4-yl)-1H-pyrrole-2-carboxylic acid (1.0 g, 3.95 mmol), HATU (2.25 g, 5.92 mmol) and DIPEA (1.55 g, 11.85 mmol) in DMF (15 mL) was stirred at rt for 30 min. After addition of tert-butyl (R)-3-(propylamino)pyrrolidine-1-carboxylate (0.90 g, 3.95 mmol) the reaction mixture was stirred at rt for 2 h. The crude material was purified by flash column chromatography (CombiFlash® system; 0-10% ethyl acetate/hexane) to provide tert-butyl (R)-3-(5-(1-phenyl-1H-pyrazol-4-yl)-N-propyl-1H-pyrrole-2-carboxamido)pyrrolidine-1-carboxylate (1.0 g, 55%) as a pale yellow oil.

LCMS [ESI, M−56]: 408.1 (RT: 2.369 min, Purity: 94.70%).

In step 4 tert-butyl (R)-3-(5-(1-phenyl-1H-pyrazol-4-yl)-N-propyl-1H-pyrrole-2-carboxamido) pyrrolidine-1-carboxylate (1.0 g, 2.15 mmol) was used. The reaction mixture was stirred at rt for 3 h. The crude material, was triturated with diethyl ether to provide (R)-5-(1-phenyl-1H-pyrazol-4-yl)-N-propyl-N-(pyrrolidin-3-yl)-1H-pyrrole-2-carboxamide hydrochloride (Compound 94: 0.650 g, 83%) as a light greenish solid.

LCMS [ESI, M+1]: 364.4 (RT: 1.460 min, Purity: 98.31%),

HPLC Purity: RT: 5.085 min, Purity: 97.74%

Chiral HPLC Purity: RT: 11.97 min, Purity: 98.79%

¹H NMR (400 MHz, D₂O): δ 8.17 (s, 1H), 7.88 (s, 1H), 7.54-7.41 (m, 4H), 7.32 (t, J=7.3 Hz, 1H), 6.45 (d, J=3.6 Hz, 1H), 6.27 (d, J=3.6 Hz, 1H), 4.35 (m, 1H), 3.70 (t, J=9.0 Hz, 1H), 3.44 (dt, J=22.0, 13.3 Hz, 4H), 3.19 (dd, J=20.6, 10.3 Hz, 1H), 2.46 (d, J=8.8 Hz, 1H), 2.19 (m, 1H), 1.71-1.47 (m, 2H), 0.84 (t, J=7.3 Hz, 3H).

### 3. Synthesis of (R)-2-(1-phenyl-1H-pyrazol-4-yl)-N-propyl-N-(pyrrolidin-3-yl)thiazole-4-carboxamide hydrochloride (Compound 171) and (S)-2-(1-phenyl-1H-pyrazol-4-yl)-N-propyl-N-(pyrrolidin-3-yl)thiazole-4-carboxamide hydrochloride (Compound 170)

### Overall Synthetic Scheme

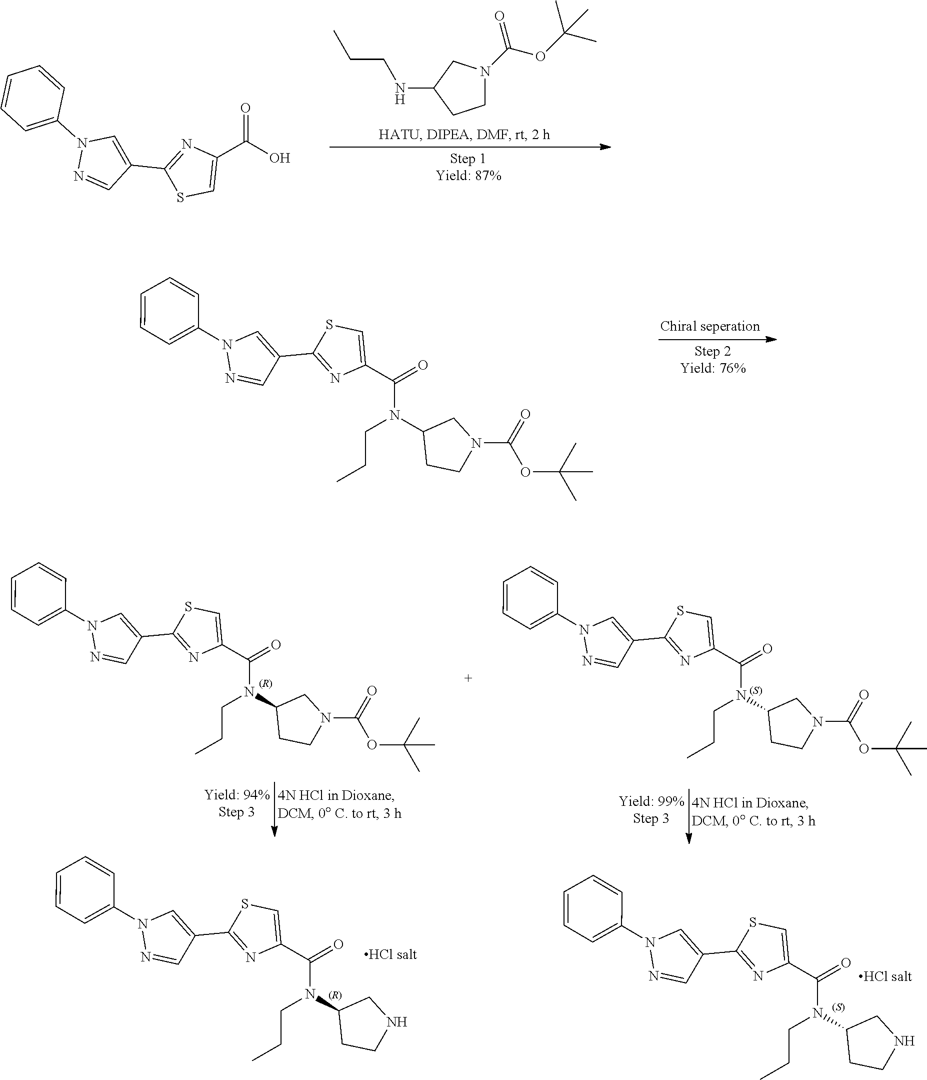

### Step 1—Synthesis of tert-butyl 3-(2-(1-phenyl-1H-pyrazol-4-yl)-N-propylthiazole-4-carboxamid o)pyrrolidine-1-carboxylate

To a stirred solution of 2-(1-phenyl-1H-pyrazol-4-yl)thiazole-4-carboxylic acid (4.0 g, 14.74 mmol) in DMF (4 mL, 10 vol) at rt. HATU (8.4 g, 22.11 mmol) and DIPEA (5.7 g, 44.23 mmol) were added. The reaction mixture was stirred for 5 min. Then tert-butyl 3-(propylamino)pyrrolidine-1-carboxylate (3.36 g, 14.74 mmol) was added to the reaction mixture at rt. The reaction mixture was stirred at rt for 2h (the progress of the reaction was monitored by TLC and LCMS analysis). After completion of the reaction, the reaction mixture was poured into cold water (80 ml) and extracted with ethyl acetate (3×40 ml). The combined organic fractions were washed with cold water 3-4 times, dried over sodium sulphate and concentrated under reduced pressure. The crude material which was purified by column chromatography (0-20% ethyl acetate/Hexane) to provide tert-butyl 3-(2-(1-phenyl-1H-pyrazol-4-yl)-N-propylthiazole-4-carboxamido)pyrrolidine-1-carboxylate(6.2 g, 87% yield) as an off-white solid.

LCMS [ESI, M+1]: 426.1[M−56] (RT: 2.349 min, Purity: 96.95%),

Chiral HPLC: R-Isomer-RT: 6.91 min, Purity: 49.30%,

S-Isomer-RT: 8.70 min, Purity: 49.21%,

¹H NMR (400 MHz, d₆-DMSO): δ 9.19 (s, 1H), 8.25 (d, J=4.5 Hz, 1H), 8.03 (s, 1H), 7.94 (d, J=7.7 Hz, 2H), 7.54 (t, J=7.8 Hz, 2H), 7.38 (t, J=7.4 Hz, 1H), 4.57 (s, 1H), 3.64 (s, 1H), 3.26 (d, J=11.8 Hz, 3H), 2.07 (s, 2H), 1.56 (s, 3H), 1.39 (s, 9H), 0.88 (s, 2H), 0.78 (d, J=7.5 Hz, 2H).

### Step 2—Synthesis of tert-butyl (R)-3-(2-(1-phenyl-1H-pyrazol-4-yl)-N-propylthiazole-4-carboxa mido)pyrrolidine-1-carboxylate and tert-butyl (S)-3-(2-(1-phenyl-1H-pyrazol-4-yl)-N-propylthiazole-4-carboxamido)pyrrolidine-1-carboxylate

Racemic tert-butyl 3-(2-(1-phenyl-1H-pyrazol-4-yl)-N-propylthiazole-4-carboxamido)pyrrolidine-1-carboxylate was purified by chiral SFC purification to isolate tert-butyl(R)-3-(2-(1-phenyl-1H-pyrazol-4-yl)-N-propylthiazole-4-carboxamido)pyrrolidine-1-carboxylate (2.4 g) and tert-butyl (S)-3-(2-(1-phenyl-1H-pyrazol-4-yl)-N-propyl thiazole-4-carboxa mido)pyrrolidine-1-carboxylate (2.3 g).

Data for tert-butyl(R)-3-(2-(1-phenyl-1H-pyrazol-4-yl)-N-propylthiazole-4-carboxamido)pyrrolidine-1-carboxylate:

LCMS [ESI, M+1]: 382.1[M−100] (RT: 2.402 min, Purity: 100%),

HPLC Purity: R-Isomer-RT: 8.97 min, Purity: 100%,

Chiral HPLC Purity: R-Isomer-RT:6.87 min, 100%,

¹H NMR (400 MHz, CD₃OD): δ 8.86 (s, 1H), 8.21 (s, 1H), 7.92 (s, 1H), 7.86 (d, J=7.1 Hz, 2H), 7.54 (t, J=7.9 Hz, 2H), 7.40 (t, J=7.4 Hz, 1H), 4.74 (s, 1H), 3.56 (dd, J=97.5, 62.6 Hz, 6H), 2.22 (s, 2H), 1.86-1.66 (m, 2H), 1.48 (s, 9H), 0.96 (s, 3H).

Data for tert-butyl (S)-3-(2-(1-phenyl-1H-pyrazol-4-yl)-N-propyl thiazole-4-carboxa mido)pyrrolidine-1-carboxylate:

LCMS [ESI, M+1]: 425.1[M−56] (RT: 2.402 min, Purity: 100%),

HPLC Purity: S-Isomer-RT: 8.974 min, Purity: 98.02%,

Chiral HPLC Purity: S-Isomer-RT:8.69 min, 97.46%,

¹H NMR (400 MHz, CD₃OD): δ 8.86 (s, 1H), 8.21 (s, 1H), 7.91 (d, J=7.5 Hz, 1H), 7.86 (d, J=7.2 Hz, 2H), 7.54 (t, J=7.9 Hz, 2H), 7.40 (t, J=7.3 Hz, 1H), 4.74 (s, 1H), 3.57 (dd, J=88.5, 57.6 Hz, 6H), 2.22 (s, 2H1), 1.74 (d, J=7.1 Hz, 2H), 1.48 (s, 9H), 0.95 (s, 3H1).

### Step 3a—Synthesis of (R)-2-(1-phenyl-1H-pyrazol-4-yl)-N-propyl-N-(pyrrolidin-3-yl)thiazole-4-carbox amide hydrochloride (Compound 171)

To a stirred solution of tert-butyl (R)-3-(2-(1-phenyl-1H-pyrazol-4-yl)-N-propylthiazole-4-carboxamido)pyrrolidine-1-carboxylate (2.4 g, 4.98 mmol) in DCM (24 ml, 10 vol) at 0° C., 4N HCl in Dioxane (4.8 ml, 2 vol) was added to the reaction mixture. The reaction mixture was stirred at rt for 3h (the progress of the reaction was monitored by TLC and LCMS analysis). After completion of the reaction, the reaction mixture was concentrated under reduced pressure to provide crude material which was purified by trituration with diethyl ether to provide (R)-2-(1-phenyl-1H-pyrazol-4-yl)-N-propyl-N-(pyrrolidin-3-yl)thiazole-4-carboxamide hydrochloride (Compound 171: 1.8 g, 94% yield) as an off-white solid.

LCMS [ESI, M+1]: 382.1 (RT: 1.417 min, Purity: 99.86%),

HPLC: RT: 4.726 min, Purity: 99.77%,

Chiral HPLC: RT: 6.00 min, Purity: 100%,

¹H NMR (400 MHz, CD₃OD): δ 8.88 (s, 1H), 8.20 (s, 1H), 8.02 (s, 1H), 7.86 (d, J=8.0 Hz, 2H), 7.55 (t, J=7.9 Hz, 2H), 7.41 (t, J=7.4 Hz, 1H), 4.36 (s, 1H), 3.77 (d, J=25.4 Hz, 1H), 3.67 (s, 4H), 3.57-3.44 (m, 1H), 3.29-3.19 (m, 1H), 2.49 (d, J=57.8 Hz, 2H), 1.82 (dd, J=14.8, 7.3 Hz, 2H), 0.93 (t, J=6.4 Hz, 3H).

### Step 3b—Synthesis of (S)-2-(1-phenyl-JH-pyrazol-4-yl)-N-propyl-N-(pyrrolidin-3-yl)thiazole-4-carboxamide hydrochloride (Compound 170)

To a stirred solution of tert-butyl (R)-3-(2-(1-phenyl-1H-pyrazol-4-yl)-N-propylthiazole-4-carboxamido)pyrrolidine-1-carboxylate (2.3 g, 4.77 mmol) in DCM (23 ml, 10 vol) at 0° C., 4N HCl in dioxane (4.6 ml, 2 vol) was added. The reaction mixture was stirred at rt for 3h (the progress of the reaction was monitored by TLC and LCMS analysis). After completion of the reaction, the reaction mixture was concentrated under reduced pressure to provide crude material which was purified by trituration with diethyl ether to provide (S)-2-(1-phenyl-1H-pyrazol-4-yl)-N-propyl-N-(pyrrolidin-3-yl)thiazole-4-carboxamide hydrochloride (Compound 170: 1.8 g, 99% yield) as an off-white solid.

LCMS [ESI, M+1]: 382.1 (RT: 1.421 min, Purity: 98.92%),

HPLC: RT: 4.720 min, Purity: 98.21%,

Chiral HPLC: RT: 6.80 min, Purity: 99.45%,

¹H NMR (400 MHz, CD₃OD): δ 8.87 (s, 1H), 8.20 (s, 1H), 8.02 (s, 1H), 7.86 (d, J=7.8 Hz, 2H), 7.55 (t, J=7.9 Hz, 2H), 7.41 (t, J=7.4 Hz, 1H), 4.36 (s, 1H), 3.77 (d, J=24.0 Hz, 1H), 3.67 (s, 3H), 3.59-3.46 (m, 1H), 3.28-3.19 (m, 1H), 2.56 (s, 1H), 2.42 (s, 1H), 1.82 (dd, J=14.9, 7.4 Hz, 2H), 0.93 (s, 3H).

### 4. Synthesis of N-isopropyl-N-(piperidin-4-yl)-2-(1-(2-(trifluoromethyl)phenyl)-1H-pyrazol-4-yl)thiazole-4-carboxamide hydrochloride (Compound 47)

### Overall Synthetic Scheme

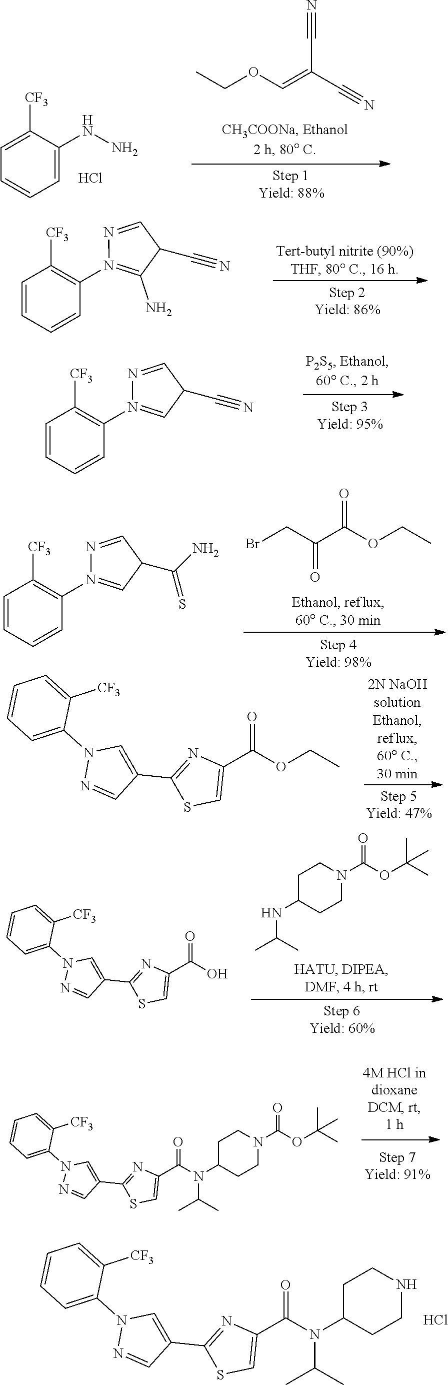

### Step-1: Synthesis of 5-amino-1-(2-(trifluoromethyl)phenyl)-1H-pyrazole-4-carbonitrile

To a stirred solution of (2-(trifluoromethyl)phenyl)hydrazine hydrochloride (4.4 g, 20.69 mmol) in ethanol (50 mL) at rt, sodium acetate (3.39 g, 41.39 mmol) and 2-(ethoxymethylene)malononitrile (2.52 g, 20.69 mmol) were added. The reaction mixture was refluxed at 80° C. for 2h (the progress of reaction was monitored by TLC and LCMS analysis). After completion of the reaction, the reaction mixture was concentrated under reduced pressure. The residual material was washed with cold water (50 mL) and extracted with ethyl acetate (3×100 mL). The combined organic fractions were dried over anhydrous sodium sulfate and concentrated under reduced pressure to provide 5-amino-1-(2-(trifluoromethyl)phenyl)-1H-pyrazole-4-carbonitrile (5.4 g, 88% yield) as a yellow solid.

LCMS [ESI, M+1]: 253.30 (RT: 1.454 min, Purity: 73.75%)

¹H NMR (400 MHz, d₆-DMSO): δ 7.85 (d, J=7.7 Hz, 1H), 7.81-7.72 (m, 1H), 7.72-7.64 (m, 2H), 7.46 (d, J=7.7 Hz, 1H), 6.59 (s, 2H).

### Step-2: Synthesis of 1-(2-(trifluoromethyl)phenyl)-1H-pyrazole-4-carbonitrile

To a stirred solution of 5-amino-1-(2-(trifluoromethyl)phenyl)-1H-pyrazole-4-carbonitrile (4.6 g, 18.24 mmol) in THF (50 mL) at rt, tert-butyl nitrite(90%)(4.13 g, 36.49 mmol) was added. The reaction mixture was refluxed at 80° C. for 16 h (the progress of the reaction was monitored by TLC and LCMS analysis). After completion of reaction, the reaction mixture was concentrated under reduced pressure. The residual material was purified by flash column chromatography (CombiFlash® system; 0-5% ethyl acetate in hexane) to provide 1-(2-(trifluoromethyl)phenyl)-1H-pyrazole-4-carbonitrile (3.7 g, 86% yield) as a yellow solid.

LCMS [ESI, M−1]:236.2(RT: 1.826 min, Purity: 89.90%)

¹H NMR (400 MHz, d₆-DMSO): δ 8.93 (s, 1H), 8.30 (s, 1H), 7.92 (d, J=7.8 Hz, 1H), 7.83 (t, J=7.4 Hz, 1H), 7.74 (t, J=7.7 Hz, 1H), 7.65 (d, J=7.8 Hz, 1H).

### Step-3: Synthesis of 1-(2-(trifluoromethyl)phenyl)-1H-pyrazole-4-carbothioamide

To a stirred solution of (1-(2-(trifluoromethyl)phenyl)-1H-pyrazole-4-carbonitrile (3.7 g, 15.5 mmol) in ethanol (37 mL) at rt, phosphorus pentasulfide (6.93 g, 31.1 mmol) was added. The reaction mixture was refluxed at 60° C. for 2 h (the progress of the reaction was monitored by TLC and LCMS analysis). After completion of the reaction, the reaction mixture was distilled under vacuum. The crude reaction mixture was washed with sodium bicarbonate solution (100 mL) until neutralization of pH and extracted with ethyl acetate (3×150 mL). The combined organic fractions were dried over sodium sulfate, concentrated under reduced pressure and triturated with diethyl ether to afford 1-(2-(trifluoromethyl)phenyl)-1H-pyrazole-4-carbothioamide (4.0 g, 95% yield) as a yellow solid.

LCMS [ESI, M+1]: 272.29(RT: 1.606 min, Purity: 80.03%)

¹H NMR (400 MHz, d₆-DMSO): δ 9.45 (s, 1H), 9.21 (s, 1H), 8.41 (s, 1H), 8.10 (s, 1H), 7.90 (t, J=8.3 Hz, 1H), 7.79 (t, J=7.7 Hz, 1H), 7.69 (t, J=7.8 Hz, 1H), 7.65-7.57 (m, 1H).

### Step—4: Synthesis of ethyl 2-(1-(2-(trifluoromethyl)phenyl)-1H-pyrazol-4-yl)thiazole-4-carboxylate

To a stirred solution of 1-(2-(trifluoromethyl)phenyl)-1H-pyrazole-4-carbothioamide (4.0 g, 14.70 mmol) in ethanol (50 mL) at rt, ethyl 3-bromo-2-oxopropanoate (3.14 g, 16.17 mmol) was added. The reaction mixture was refluxed at 60° C. for 30 min (the progress of the reaction was monitored by TLC and LCMS analysis). After completion of the reaction, the reaction mixture was distilled under vacuum and triturated with diethyl ether to provide ethyl 2-(1-(2-(trifluoromethyl)phenyl)-1H-pyrazol-4-yl)thiazole-4-carboxylate (5.3 g, 98% yield) as a yellow solid.

LCMS [ESI, M+1]: 367.9(RT: 2.015 min, Purity: 72.00%)

¹H NMR (400 MHz, d₆-DMSO): δ 8.75 (s, 1H), 8.39 (d, J=13.9 Hz, 1H), 8.23 (s, 1H), 7.91 (d, J=7.8 Hz, 1H), 7.85-7.75 (m, 1H), 7.72 (d, J=7.3 Hz, 1H), 7.67 (d, J=7.8 Hz, 1H), 4.24 (dd, J=14.2, 7.1 Hz, 2H), 1.28-1.19 (t, J=7.2 Hz, 3H).

### Step-5: Synthesis of 2-(1-(2-(trifluoromethyl)phenyl)-1H-pyrazol-4-yl)thiazole-4-carboxylic Acid

To a stirred solution of ethyl 2-(1-(2-(trifluoromethyl)phenyl)-1H-pyrazol-4-yl)thiazole-4-carboxylate (5.3 g, 14.4 mmol) in ethanol (60 mL) at rt, 2 N NaOH solution (21.62 mL) was added. The reaction mixture was heated at 60° C. for 30 minutes (the progress of the reaction was monitored by TLC and LCMS analysis). After completion of the reaction, the reaction mixture was poured into water (50 mL) and extracted with ethyl acetate (3×50 mL). The aqueous layer was separated out and acidified with 2 M HCl solution to generated a precipitate, which was isolated by filtration and dried under reduced pressure to provide 2-(1-(2-(trifluoromethyl)phenyl)-1H-pyrazol-4-yl)thiazole-4-carboxylic acid (2.3 g, 47% yield) as a brown solid.

LCMS [ESI, M+1]: 339.85 (RT: 1.613 min, Purity: 99.13%)

HPLC: 6.280 min, 99.63%

¹H NMR (400 MHz, d₆-DMSO): δ 13.01 (s, 1H), 8.69 (d, J=21.4 Hz, 1H), 8.44-8.28 (m, 1H), 8.18 (d, J=21.6 Hz, 1H), 7.91 (d, J=7.7 Hz, 1H), 7.81 (t, J=7.5 Hz, 1H), 7.67 (dt, J=30.1, 15.0 Hz, 2H).

### Step-6: Synthesis of tert-butyl-4-(N-isopropyl-2-(1-(2-(trifluoromethyl)phenyl)-1H-pyrazol-4-yl) thiazole-4-carboxamido)piperidine-1-carboxylate

To a stirred solution of 2-(1-(2-(trifluoromethyl)phenyl)-1H-pyrazol-4-yl)thiazole-4-carboxylic acid (0.20 g, 0.58 mmol) in DMF (4 mL) at rt, HATU (0.33 g, 0.87 mmol) and DIPEA (0.21 g, 1.7 mmol) were added. The reaction mixture was stirred for 30 min and then tert-butyl 4-(isopropylamino)piperidine-1-carboxylate (0.142 g, 0.58 mmol) was added at rt. The reaction mixture was stirred at rt for 4h (the progress of the reaction was monitored by TLC and LCMS analysis). After completion of the reaction, the reaction mixture was poured into cold water (15 mL) and extracted with ethyl acetate (3×50 mL). The combined organic fractions were washed with cold water 3-4 times, dried over sodium sulfate and concentrated under reduced pressure. The crude material was purified by flash column chromatography (CombiFlash® system; 18-22% ethyl acetate in Hexane) to provie tert-butyl4-(N-isopropyl-2-(1-(2-(trifluoromethyl)phenyl)-1H-pyrazol-4-yl)thiazole-4-carboxamido)piperidine-1-carboxylate (0.2 g, 60% yield) as a white solid.

LCMS [ESI, M−56]: 507.98(RT: 2.388 min, Purity: 97.26%)

### Step-7: Synthesis of N-isopropyl-N-(piperidin-4-yl)-2-(1-(2-(trifluoromethyl)phenyl)-1H-pyrazol-4-yl)thiazole-4-carboxamide hydrochloride (Compound 47)

To a stirred solution of tert-butyl4-(N-isopropyl-2-(1-(2-(trifluoromethyl)phenyl)-1H-pyrazol-4-yl)thiazole-4-carboxamid o)piperidine-1-carboxylate (0.20 g, 0.35 mmol) in DCM (2 mL, 10V) at 0° C., 4M HCl in Dioxane (1 mL, 5V) was added. The reaction mixture was stirred at room temperature for 1h (the progress of the reaction was monitored by TLC and LCMS analysis). After completion of reaction, the reaction mixture was concentrated under reduced pressure, and the crude material triturated with diethyl ether to provide (N-isopropyl-N-(piperidin-4-yl)-2-(1-(2-(trifluoromethyl)phenyl)-1H-pyrazol-4-yl)thiazole-4-carboxamide hydro chloride (Compound 47: 0.150 g, 91% yield), as an off-white sticky solid.

LCMS [ESI, M+1]: 464.07(RT: 1.411 min, Purity: 98.37%)

HPLC: RT: 4.758 min, Purity: 97.90%

¹H NMR (400 MHz, CD₃OD): δ 8.53 (s, 1H1), 8.25 (s, 1H1), 7.96 (d, J=7.8 Hz, 1H1), 7.91-7.73 (m, 3H), 7.68 (d, J=7.8 Hz, 1H1), 4.26 (s, 1H), 3.66 (d, J=17.7 Hz, 1H1), 3.51 (d, J=10.5 Hz, 2H1), 3.24-2.99 (in, 3H1), 1.96 (s, 2H1), 1.55 (s, 1H), 1.25 (d, J=74.4 Hz, 6H1).

TABLE 1
Compounds 5-210 with chemical name, LCMS data and experimental procedure

| | | | Observed | | Exp. |
|---|---|---|---|---|---|
| Comp. | | | MW in | Salt | Proc. |
| No. | Chemical Name | MW | LCMS | Form | No. |
| 5 | 5-(1-phenyl-1H-pyrazol-4-yl)-N- | 377 | 378 | HCl | 3 |
| | (piperidin-4-yl)-N-(propan-2-yl)-1H- | | | salt | |
| | pyrrole-2-carboxamide | | | | |
| 6 | 2-[1-(4-fluorophenyl)-1H- | 413 | 414 | HCl | |
| | pyrazol-4-yl]-N-(piperidin-4-yl)- | | | salt | |
| | N-(propan-2-yl)-1,3-thiazole-4- | | | | |
| | carboxamide | | | | |
| 8 | N-propyl-2-[1-(pyridin-3-yl)-1H- | 382 | 383 | HCl | 4 |
| | pyrazol-4-yl]-N-[(3R)-pyrrolidin-3-yl]- | | | salt | |
| | 1,3-thiazole-4-carboxamide | | | | |
| 9 | N-propyl-2-[1-(pyridin-4-yl)-1H- | 382 | 383 | HCl | 4 |
| | pyrazol-4-yl]-N-[(3S)-pyrrolidin-3-yl]- | | | salt | |
| | 1,3-thiazole-4-carboxamide | | | | |
| 11 | 1-[(2R)-1-[5-(1-phenyl-1H- | 336 | 337 | HCl | 3 |
| | pyrazol-4-yl)furan-2- | | | salt | |
| | carbonyl]pyrrolidin-2- | | | | |
| | yl]methanamine | | | | |
| 12 | N-(2-aminoethyl)-2-(2-phenyl- | 314 | 315 | HCl | 3 |
| | 1,3-oxazol-5-yl)-1,3-thiazole-4- | | | Salt | |
| | carboxamide | | | | |
| 14 | 1-[(2R)-1-[2-(2-phenyl-1,3- | 354 | 355 | HCl | 3 |
| | oxazol-5-yl)-1,3-thiazole-4- | | | Salt | |
| | carbonyl]pyrrolidin-2-yl]methanamine | | | | |
| 16 | 4-(1-phenyl-1H-pyrazol-4-yl)-N- | 352 | 353 | HCl | 3 |
| | {[(3R)-pyrrolidin-3- | | | Salt | |
| | yl]methyl}thiophene-2-carboxamide | | | | |
| 17 | N-(5-aminopentyl)-4-(1-phenyl-1H- | 354 | 355 | HCl | 3 |
| | pyrazol-4-yl)thiophene-2- | | | Salt | |
| | carboxamide | | | | |
| 18 | N-(2-aminoethyl)-4-(1-phenyl- | 312 | 313 | HCl | 3 |
| | 1H-pyrazol-4-yl)thiophene-2- | | | Salt | |
| | carboxamide | | | | |
| 21 | N-(5-aminopentyl)-5-(1-phenyl-1H- | 337 | 338 | HCl | 3 |
| | pyrazol-4-yl)-1H-pyrrole-2- | | | salt | |
| | carboxamide | | | | |
| 22 | N-propyl-2-[1-(pyridin-3-yl)-1H- | 382 | 383 | HCl | 4 |
| | pyrazol-4-yl]-N-[(3S)-pyrrolidin-3-yl]- | | | salt | |
| | 1,3-thiazole-4-carboxamide | | | | |
| 23 | N-propyl-2-[1-(pyridin-4-yl)-1H- | 382 | 383 | HCl | 4 |
| | pyrazol-4-yl]-N-[(3R)-pyrrolidin-3-yl]- | | | salt | |
| | 1,3-thiazole-4-carboxamide | | | | |
| 25 | N-propyl-N-[(3S)-pyrrolidin-3-yl]-2- | 465 | 1.71 | HCl | 4 |
| | {1-[3- | | | salt | |
| | (trifluoromethoxy)phenyl]-1H- | | | | |
| | pyrazol-4-yl}-1,3-thiazole-4- | | | | |
| | carboxamide | | | | |
| 26 | N-propyl-N-[(3R)-pyrrolidin-3- yl]-2- | 465 | 1.72 | HCl | 4 |
| | {1-[3- | | | salt | |
| | (trifluoromethoxy)phenyl]-1H-pyrazol- | | | | |
| | 4-yl}-1,3-thiazole-4-carboxamide | | | | |
| 28 | 2-[1-(3-methylphenyl)-1H- | 395 | 396 | HCl | 4 |
| | pyrazol-4-yl]-N-propyl-N-[(3S)- | | | Salt | |
| | pyrrolidin-3-yl]-1,3-thiazole-4- | | | | |
| | carboxamide | | | | |
| 29 | 2-[1-(3-methylphenyl)-1H- | 395 | 396 | HCl | 4 |
| | pyrazol-4-yl]-N-propyl-N-[(3R)- | | | Salt | |
| | pyrrolidin-3-yl]-1,3-thiazole-4- | | | | |
| | carboxamide | | | | |
| 31 | 2-[1-(3-chlorophenyl)-1H- | 415 | 416 | HCl | 4 |
| | pyrazol-4-yl]-N-propyl-N-[(3S)- | | | salt | |
| | pyrrolidin-3-yl]-1,3-thiazole-4- | | | | |
| | carboxamide | | | | |
| 32 | 2-[1-(3-chlorophenyl)-1H- | 415 | 416 | HCl | 4 |
| | pyrazol-4-yl]-N-propyl-N-[(3R)- | | | salt | |
| | pyrrolidin-3-yl]-1,3-thiazole-4- | | | | |
| | carboxamide | | | | |
| 35 | 2-(2-phenyl-1,3-oxazol-5-yl)-N- | 382 | 383 | HCl | 3 |
| | propyl-N-[(3S)-pyrrolidin-3-yl]-1,3- | | | salt | |
| | thiazole-4-carboxamide | | | | |
| 36 | 5-(1-phenyl-1H-pyrazol-4-yl)-N- | 380 | 381 | HCl | 3 |
| | propyl-N-[(3R)-pyrrolidin-3- | | | Salt | |
| | yl]thiophene-3-carboxamide | | | | |
| 40 | 2-[1-(4-chlorophenyl)-1H- | 416 | 416 | HCl | 4 |
| | pyrazol-4-yl]-N-propyl-N-[(3S)- | | | salt | |
| | pyrrolidin-3-yl]-1,3-thiazole-4- | | | | |
| | carboxamide | | | | |
| 41 | 2-[1-(4-chlorophenyl)-1H- | 416 | 416 | HCl | 4 |
| | pyrazol-4-yl]-N-propyl-N-[(3R)- | | | salt | |
| | pyrrolidin-3-yl]-1,3-thiazole-4- | | | | |
| | carboxamide | | | | |
| 42 | 5-(1-phenyl-1H-pyrazol-4-yl)-N- | 380 | 381 | HCl | 3 |
| | propyl-N-[(3S)-pyrrolidin-3- | | | Salt | |
| | yl]thiophene-3-carboxamide | | | | |
| 43 | N-propyl-2-[1-(pyridin-2-yl)-1H- | 382 | 383 | HCl | 4 |
| | pyrazol-4-yl]-N-[(3S)-pyrrolidin- 3-yl]- | | | salt | |
| | 1,3-thiazole-4-carboxamide | | | | |
| 44 | N-propyl-2-[1-(pyridin-2-yl)-1H- | 382 | 383 | HCl | 4 |
| | pyrazol-4-yl]-N-[(3R)-pyrrolidin-3-yl]- | | | salt | |
| | 1,3-thiazole-4-carboxamide | | | | |
| 45 | N-propyl-N-[(3S)-pyrrolidin-3-yl]-2- | 466 | 466 | HCl | 4 |
| | {1-[4- | | | salt | |
| | (trifluoromethoxy)phenyl]-1H- pyrazol- | | | | |
| | 4-yl}-1,3-thiazole-4-carboxamide | | | | |
| 46 | N-propyl-N-[(3R)-pyrrolidin-3-yl]-2- | 466 | 466 | HCl | 4 |
| | {1-[4- | | | salt | |
| | (trifluoromethoxy)phenyl]-1H-pyrazol- | | | | |
| | 4-yl}-1,3-thiazole-4-carboxamide | | | | |
| 47 | N-(piperidin-4-yl)-N-(propan-2-yl)-2- | 464 | 464 | HCl | see full |
| | {1-[2- | | | salt | experimental |
| | (trifluoromethyl)phenyl]-1H- | | | | |
| | pyrazol-4-yl}-1,3-thiazole-4- | | | | |
| | carboxamide | | | | |
| 48 | N-propyl-N-[(3S)-pyrrolidin-3-yl]-2- | 450 | 450 | HCl | 4 |
| | {1-[3- | | | Salt | |
| | (trifluoromethyl)phenyl]-1H-pyrazol-4- | | | | |
| | yl}-1,3-thiazole-4-carboxamide | | | | |
| 49 | N-propyl-N-[(3R)-pyrrolidin-3-yl]-2- | 450 | 450 | HCl | 4 |
| | {1-[3- | | | Salt | |
| | (trifluoromethyl)phenyl]-1H-pyrazol-4- | | | | |
| | yl}-1,3-thiazole-4-carboxamide | | | | |
| 50 | 1-[(2R)-1-{2-[1-(3- | 383 | 384 | HCl | 4 |
| | methoxyphenyl)-1H-pyrazol-4-yl]-1,3- | | | salt | |
| | thiazole-4-carbonyl}pyrrolidin-2- | | | | |
| | yl]methanamine | | | | |
| 51 | 2-[1-(3-methoxyphenyl)-1H- | 412 | 412 | HCl | 4 |
| | pyrazol-4-yl]-N-propyl-N-[(3S)- | | | salt | |
| | pyrrolidin-3-yl]-1,3-thiazole-4- | | | | |
| | carboxamide | | | | |
| 52 | 2-[1-(3-fluorophenyl)-1H- | 399 | 400 | HCl | 4 |
| | pyrazol-4-yl]-N-propyl-N-[(3S)- | | | salt | |
| | pyrrolidin-3-yl]-1,3-thiazole-4- | | | | |
| | carboxamide | | | | |
| 53 | 2-[1-(3-fluorophenyl)-1H- | 399 | 400 | HCl | 4 |
| | pyrazol-4-yl]-N-propyl-N-[(3R)- | | | salt | |
| | pyrrolidin-3-yl]-1,3-thiazole-4- | | | | |
| | carboxamide | | | | |
| 54 | 2-[1-(4-fluorophenyl)-1H- | 399 | 400 | HCl | 4 |
| | pyrazol-4-yl]-N-propyl-N-[(3R)- | | | salt | |
| | pyrrolidin-3-yl]-1,3-thiazole-4- | | | | |
| | carboxamide | | | | |
| 56 | 2-[1-(3-methoxyphenyl)-1H- | 412 | 412 | HCl | 4 |
| | pyrazol-4-yl]-N-propyl-N-[(3R)- | | | salt | |
| | pyrrolidin-3-yl]-1,3-thiazole-4- | | | | |
| | carboxamide | | | | |
| 57 | 2-[1-(4-methylphenyl)-1H- | 410 | 410 | HCl | 4 |
| | pyrazol-4-yl]-N-(piperidin-4-yl)- | | | Salt | |
| | N-(propan-2-yl)-1,3-thiazole-4- | | | | |
| | carboxamide | | | | |
| 58 | 2-[1-(4-fluorophenyl)-1H- | 399 | 400 | HCl | 4 |
| | pyrazol-4-yl]-N-propyl-N-[(3S)- | | | salt | |
| | pyrrolidin-3-yl]-1,3-thiazole-4- | | | | |
| | carboxamide | | | | |
| 59 | 4-(1-phenyl-1H-pyrazol-4-yl)-N- | 380 | 381 | HCl | 3 |
| | propyl-N-[(3S)-pyrrolidin-3- | | | Salt | |
| | yl]thiophene-2-carboxamide | | | | |
| 61 | 2-[1-(3-methoxyphenyl)-1H- | 426 | 426 | HCl | 4 |
| | pyrazol-4-yl]-N-(piperidin-4-yl)- | | | Salt | |
| | N-(propan-2-yl)-1,3-thiazole-4- | | | | |
| | carboxamide | | | | |
| 64 | 2-[1-(4-methylphenyl)-1H- | 396 | 396 | HCl | 4 |
| | pyrazol-4-yl]-N-propyl-N-[(3S)- | | | salt | |
| | pyrrolidin-3-yl]-1,3-thiazole-4- | | | | |
| | carboxamide | | | | |
| 65 | 2-[1-(4-methylphenyl)-1H- | 396 | 396 | HCl | 4 |
| | pyrazol-4-yl]-N-propyl-N-[(3R)- | | | salt | |
| | pyrrolidin-3-yl]-1,3-thiazole-4- | | | | |
| | carboxamide | | | | |
| 66 | 2-[1-(2-chlorophenyl)-1H- | \|430 | 430 | HCl | 4 |
| | pyrazol-4-yl]-N-(piperidin-4-yl)- | | | salt | |
| | N-(propan-2-yl)-1,3-thiazole-4- | | | | |
| | carboxamide | | | | |
| 68 | 1-[(2R)-1-[5-(1-phenyl-1H- | 335 | 336 | HCl | 3 |
| | pyrazol-4-yl)-1H-pyrrole-2- | | | salt | |
| | carbonyl]pyrrolidin-2-yl]methanamine | | | | |
| 69 | 2-(5-phenylthiophen-2-yl)-N- | 398 | 398 | HCl | 3 |
| | propyl-N-[(3S)-pyrrolidin-3-yl]-1,3- | | | Salt | |
| | thiazole-4-carboxamide | | | | |
| 70 | 2-(5-phenylthiophen-2-yl)-N-propyl-N- | 398 | 398 | HCl | 3 |
| | [(3R)-pyrrolidin-3-yl]- | | | Salt | |
| | 1,3-thiazole-4-carboxamide | | | | |
| 71 | 2-(1-phenyl-1H-pyrazol-4-yl)-N- | 382 | 382 | HCl | 2 |
| | (propan-2-yl)-N-(pyrrolidin-3-yl)-1,3- | | | Salt | |
| | thiazole-4-carboxamide | | | | |
| 73 | 1-[(2R)-1-(2-{1-[2- | 421 | 422 | HCl | 4 |
| | (trifluoromethyl)phenyl]-1H-pyrazol-4- | | | salt | |
| | yl}-1,3-thiazole-4-carbonyl)pyrrolidin- | | | | |
| | 2-yl]methanamine | | | | |
| 74 | N-(piperidin-4-yl)-N-(propan-2-yl)-2- | 464 | 464 | HCl | 4 |
| | {1-[4- | | | Salt | |
| | (trifluoromethyl)phenyl]-1H-pyrazol-4- | | | | |
| | yl}-1,3-thiazole-4-carboxamide | | | | |
| 76 | 1-[(2R)-1-(2-{1-[4- | 421 | 422 | HCl | 4 |
| | (trifluoromethyl)phenyl]-1H-pyrazol-4- | | | Salt | |
| | yl}-1,3-thiazole-4-carbonyl)pyrrolidin- | | | | |
| | 2-yl]methanamine | | | | |
| 77 | N-propyl-N-[(3S)-pyrrolidin-3-yl]-2- | 450 | 450 | HCl | 4 |
| | {1-[4- | | | Salt | |
| | (trifluoromethyl)phenyl]-1H- pyrazol-4- | | | | |
| | yl}-1,3-thiazole-4-carboxamide | | | | |
| 78 | N-propyl-N-[(3R)-pyrrolidin-3-yl]-2- | 450 | 450 | HCl | 4 |
| | {1-[4-(trifluoromethyl)phenyl]-1H- | | | Salt | |
| | pyrazol-4-yl}-1,3-thiazole-4- | | | | |
| | carboxamide | | | | |
| 80 | 1-[(2R)-1-{2-[1-(2- | 388 | 388 | HCl | 4 |
| | chlorophenyl)-1H-pyrazol-4-yl]-1,3- | | | salt | |
| | thiazole-4-carbonyl}pyrrolidin-2- | | | | |
| | yl]methanamine | | | | |
| 81 | 4-(1-phenyl-1H-pyrazol-4-yl)-N- | 380 | 381 | HCl | 3 |
| | propyl-N-[(3R)-pyrrolidin-3- | | | Salt | |
| | yl]thiophene-2-carboxamide | | | | |
| 82 | N-propyl-N-[(3S)-pyrrolidin-3-yl]-2- | 450 | 450 | HCl | 4 |
| | {1-[2-(trifluoromethyl)phenyl]-1H- | | | salt | |
| | pyrazol-4-yl}-1,3-thiazole-4- | | | | |
| | carboxamide | | | | |
| 83 | N-propyl-N-[(3R)-pyrrolidin-3-yl]-2- | 450 | 450 | HCl | 4 |
| | {1-[2-(trifluoromethyl)phenyl]-1H- | | | salt | |
| | pyrazol-4-yl}-1,3-thiazole-4- | | | | |
| | carboxamide | | | | |
| 84 | 2-[1-(2-methylphenyl)-1H- | 410 | 410 | HCl | 4 |
| | pyrazol-4-yl]-N-(piperidin-4-yl)- | | | Salt | |
| | N-(propan-2-yl)-1,3-thiazole-4- | | | | |
| | carboxamide | | | | |
| 86 | 1-[(2R)-1-{2-[1-(2- | 367 | 368 | HCl | 4 |
| | methylphenyl)-1H-pyrazol-4-yl]-1,3- | | | Salt | |
| | thiazole-4-carbonyl}pyrrolidin-2- | | | | |
| | yl]methanamine | | | | |
| 87 | 2-[1-(2-methylphenyl)-1H- | 396 | 396 | HCl | 4 |
| | pyrazol-4-yl]-N-propyl-N-[(3S)- | | | Salt | |
| | pyrrolidin-3-yl]-1,3-thiazole-4- | | | | |
| | carboxamide | | | | |
| 88 | 2-[1-(2-methylphenyl)-1H- | 396 | 396 | HCl | 4 |
| | pyrazol-4-yl]-N-propyl-N-[(3R)- | | | Salt | |
| | pyrrolidin-3-yl]-1,3-thiazole-4- | | | | |
| | carboxamide | | | | |
| 89 | 2-[1-(2-chlorophenyl)-1H- | 416 | 416 | HCl | 4 |
| | pyrazol-4-yl]-N-propyl-N-[(3S)- | | | salt | |
| | pyrrolidin-3-yl]-1,3-thiazole-4- | | | | |
| | carboxamide | | | | |
| 90 | 2-[1-(2-chlorophenyl)-1H- | 416 | 416 | HCl | 4 |
| | pyrazol-4-yl]-N-propyl-N-[(3R)- | | | salt | |
| | pyrrolidin-3-yl]-1,3-thiazole-4- | | | | |
| | carboxamide | | | | |
| 91 | 1-[(2R)-1-{2-[1-(2- | 371 | 372 | HCl | 4 |
| | fluorophenyl)-1H-pyrazol-4-yl]-1,3- | | | salt | |
| | thiazole-4-carbonyl}pyrrolidin-2- | | | | |
| | yl]methanamine | | | | |
| 92 | 2-[1-(2-fluorophenyl)-1H- | 399 | 400 | HCl | 4 |
| | pyrazol-4-yl]-N-propyl-N-[(3S)- | | | salt | |
| | pyrrolidin-3-yl]-1,3-thiazole-4- | | | | |
| | carboxamide | | | | |
| 93 | 5-(1-phenyl-1H-pyrazol-4-yl)-N- | 363 | 364 | HCl | 3 |
| | propyl-N-[(3S)-pyrrolidin-3-yl]-1H- | | | salt | |
| | pyrrole-2-carboxamide | | | | |
| 94 | 5-(1-phenyl-1H-pyrazol-4-yl)-N- | 363 | 364 | HCl | see full |
| | propyl-N-[(3R)-pyrrolidin-3-yl]-1H- | | | salt | experimental |
| | pyrrole-2-carboxamide | | | | |
| 95 | 2-[1-(2-fluorophenyl)-1H- | 414 | 414 | HCl | 4 |
| | pyrazol-4-yl]-N-(piperidin-4-yl)- | | | salt | |
| | N-(propan-2-yl)-1,3-thiazole-4- | | | | |
| | carboxamide | | | | |
| 97 | 2-[1-(2-fluorophenyl)-1H- | 399 | 400 | HCl | 4 |
| | pyrazol-4-yl]-N-propyl-N-[(3R)- | | | salt | |
| | pyrrolidin-3-yl]-1,3-thiazole-4- | | | | |
| | carboxamide | | | | |
| 98 | 5-(1-phenyl-1H-pyrazol-4-yl)-N- | 364 | 365 | HCl | 3 |
| | propyl-N-[(3S)-pyrrolidin-3-yl]furan-2- | | | Salt | |
| | carboxamide | | | | |
| 99 | 5-(1-phenyl-1H-pyrazol-4-yl)-N- | 364 | 365 | HCl | 3 |
| | propyl-N-[(3R)-pyrrolidin-3-yl]furan- | | | Salt | |
| | 2-carboxamide | | | | |
| 100 | 5-(1-phenyl-1H-pyrazol-4-yl)-N- | 350 | 351 | HCl | 3 |
| | [(piperidin-4-yl)methyl]furan-2- | | | Salt | |
| | carboxamide | | | | |
| 101 | N-[2-(2-aminoethoxy)ethyl]-5-(1 | 340 | 341 | HCl | 3 |
| | phenyl-1H-pyrazol-4-yl)furan-2- | | | Salt | |
| | carboxamide | | | | |
| 104 | 1-[2-(1-phenyl-1H-pyrazol-4-yl)- | 339 | 340 | HCl | 2 |
| | 1,3-thiazole-4-carbonyl]piperazine | | | Salt | |
| 106 | N-{[(2R)-morpholin-2- | 369 | 370 | HCl | 2 |
| | yl]methyl}-2-(1-phenyl-1H-pyrazol-4- | | | salt | |
| | yl)-1,3-thiazole-4-carboxamide | | | | |
| 107 | N-{[(2S)-morpholin-2- | 369 | 370 | HCl | 2 |
| | yl]methyl}-2-(1-phenyl-1H-pyrazol-4- | | | salt | |
| | yl)-1,3-thiazole-4-carboxamide | | | | |
| 108 | 2-(1-phenyl-1H-pyrazol-4-yl)-N- | 353 | 354 | HCl | 2 |
| | {[(3S)-pyrrolidin-3-yl]methyl}-1,3- | | | salt | |
| | thiazole-4-carboxamide | | | | |
| 110 | N-[2-(2-aminoethoxy)ethyl]-4-(1 | 356 | 357 | HCl | 3 |
| | phenyl-1H-pyrazol-4- | | | Salt | |
| | yl)thiophene-2-carboxamide | | | | |
| 111 | 4-(1-phenyl-1H-pyrazol-4-yl)-N- | 366 | 367 | HCl | 3 |
| | [(piperidin-4-yl)methyl]thiophene-2- | | | Salt | |
| | carboxamide | | | | |
| 112 | 2-(1-phenyl-1H-pyrazol-4-yl)-N- | 353 | 354 | HCl | 2 |
| | {[(3R)-pyrrolidin-3-yl]methyl}-1,3- | | | salt | |
| | thiazole-4-carboxamide | | | | |
| 114 | 1-[(2R)-1-[4-(1-phenyl-1H- | 352 | 353 | HCl | 3 |
| | pyrazol-4-yl)thiophene-2- | | | Salt | |
| | carbonyl]pyrrolidin-2-yl]methanamine | | | | |
| 115 | 4-(5-phenyl-1,3,4-thiadiazol-2-yl)-N- | 398 | 399 | HCl | 3 |
| | propyl-N-(pyrrolidin-3- | | | Salt | |
| | yl)thiophene-2-carboxamide | | | | |
| 116 | 2-(2-phenyl-1,3-oxazol-5-yl)-N-propyl- | 382 | 383 | HCl | 3 |
| | N-(pyrrolidin-3-yl)-1,3- | | | Salt | |
| | thiazole-4-carboxamide | | | | |
| 117 | N-[(1R,3R)-3-aminocyclopentyl] 2-(1- | 353 | 354 | HCl | 2 |
| | phenyl-1H-pyrazol-4-yl)-1,3-thiazole- | | | Salt | |
| | 4-carboxamide | | | | |
| 119 | 1-{1-[2-(1-phenyl-1H-pyrazol-4-yl)- | 339 | 340 | Methan | 2 |
| | 1,3-thiazole-4-carbonyl]azetidin-3- | | | sulfonic | |
| | yl}methanamine | | | acid | |
| | | | | salt | |
| 120 | 5-(1-phenyl-1H-pyrazol-4-yl)-N- | 363 | 364 | HCl | 3 |
| | propyl-N-(pyrrolidin-3-yl)-1H- | | | Salt | |
| | pyrrole-2-carboxamide | | | | |
| 121 | 1-{1-[2-(1-phenyl-1H-pyrazol-4-yl)- | 353 | 354 | HCl | 2 |
| | 1,3-thiazole-4-carbonyl]pyrrolidin-3- | | | Salt | |
| | yl}methanamine | | | | |
| 122 | 2-(5-phenylthiophen-2-yl)-N-propyl-N- | 398 | 398 | HCl | 3 |
| | (pyrrolidin-3-yl)-1,3- | | | Salt | |
| | thiazole-4-carboxamide | | | | |
| 123 | 4-(1-phenyl-1H-pyrazol-4-yl)-N- | 363 | 364 | HCl | 3 |
| | propyl-N-(pyrrolidin-3-yl)-1H- | | | Salt | |
| | pyrrole-2-carboxamide | | | | |
| 124 | 1-[2-(1-phenyl-1H-pyrazol-4-yl)-1,3- | 325 | 326 | HCl | 2 |
| | thiazole-4-carbonyl]azetidin- | | | Salt | |
| | 3-amine | | | | |
| 126 | 4-(5-phenyl-1,3,4-oxadiazol-2-yl)-N- | 382 | 383 | HCl | 3 |
| | propyl-N-(pyrrolidin-3- | | | salt | |
| | yl)thiophene-2-carboxamide | | | | |
| 127 | 4-(1-phenyl-1H-pyrazol-4-yl)-N- | 382 | 382 | HCl | 2 |
| | propyl-N-(pyrrolidin-3-yl)-1,3- | | | Salt | |
| | thiazole-2-carboxamide | | | | |
| 128 | N-[(2R)-2-aminopropyl]-2-(1- | 327 | 328 | HCl | 2 |
| | phenyl-1H-pyrazol-4-yl)-1,3-thiazole- | | | Salt | |
| | 4-carboxamide | | | | |
| 129 | N-[(2R)-1-aminopropan-2-yl]-2- | 327 | 328 | HCl | 2 |
| | (1-phenyl-1H-pyrazol-4-yl)-1,3- | | | Salt | |
| | thiazole-4-carboxamide | | | | |
| 130 | 5-(1-phenyl-1H-pyrazol-4-yl)-N- | 380 | 381 | HCl | 3 |
| | propyl-N-(pyrrolidin-3-yl)thiophene-3- | | | Salt | |
| | carboxamide | | | | |
| 131 | 1-[(2S)-1-[2-(1-phenyl-1H- | 353 | 354 | HCl | 2 |
| | pyrazol-4-yl)-1,3-thiazole-4- | | | Salt | |
| | carbonyl]pyrrolidin-2-yl]methanamine | | | | |
| 132 | 2-(1-phenyl-1H-pyrazol-4-yl)-N- | 353 | 354 | HCl | 2 |
| | (piperidin-3-yl)-1,3-thiazole-4- | | | Salt | |
| | carboxamide | | | | |
| 133 | N-cyclobutyl-2-(1-phenyl-1H- | 408 | 408 | HCl | 2 |
| | pyrazol-4-yl)-N-(piperidin-3-yl)-1,3- | | | Salt | |
| | thiazole-4-carboxamide | | | | |
| 135 | 2-(1-phenyl-1H-pyrazol-4-yl)-N- | 365 | 366 | HCl | 3 |
| | propyl-N-(pyrrolidin-3-yl)-1,3- | | | Salt | |
| | oxazole-4-carboxamide | | | | |
| 136 | 4-(1-phenyl-1H-pyrazol-4-yl)-N- | 364 | 365 | HCl | 3 |
| | propyl-N-(pyrrolidin-3-yl)furan- | | | Salt | |
| | 2-carboxamide | | | | |
| 137 | 1-[(3S)-1-[2-(1-phenyl-1H- | 353 | 354 | HCl | 2 |
| | pyrazol-4-yl)-1,3-thiazole-4- | | | Salt | |
| | carbonyl]pyrrolidin-3-yl]methanamine | | | | |
| 139 | N-methyl-2-(1-phenyl-1H- | 367 | 368 | HCl | 2 |
| | pyrazol-4-yl)-N-(piperidin-3-yl)-1,3- | | | Salt | |
| | thiazole-4-carboxamide | | | | |
| 140 | 5-(1-phenyl-1H-pyrazol-4-yl)-N- | 364 | 365 | HCl | 3 |
| | propyl-N-(pyrrolidin-3-yl)furan-2- | | | Salt | |
| | carboxamide | | | | |
| 141 | 4-(1-phenyl-1H-pyrazol-4-yl)-N- | 380 | 381 | HCl | 3 |
| | propyl-N-(pyrrolidin-3- | | | Salt | |
| | yl)thiophene-2-carboxamide | | | | |
| 142 | (3S)-1-[2-(1-phenyl-1H-pyrazol-4-yl)- | 353 | 354 | HCl | 2 |
| | 1,3-thiazole-4- | | | Salt | |
| | carbonyl]piperidin-3-amine | | | | |
| 143 | (3R)-1-[2-(1-phenyl-1H-pyrazol-4-yl)- | 353 | 354 | HCl | 2 |
| | 1,3-thiazole-4- | | | Salt | |
| | carbonyl]piperidin-3-amine | | | | |
| 145 | N-(2-amino-2-methylpropyl)-2-(1- | 341 | 342 | HCl | commercially |
| | phenyl-1H-pyrazol-4-yl)-1,3- | | | Salt | available |
| | thiazole-4-carboxamide | | | | |
| 146 | 2-(1-phenyl-1H-pyrazol-4-yl)-N- | 339 | 340 | HCl | 2 |
| | [(1r,3r)-3-aminocyclobutyl]-1,3- | | | Salt | |
| | thiazole-4-carboxamide | | | | |
| 149 | N-ethyl-2-(1-phenyl-1H-pyrazol-4-yl)- | 382 | 382 | HCl | 2 |
| | N-(piperidin-4-yl)-1,3-thiazole-4- | | | Salt | |
| | carboxamide | | | | |
| 150 | N-ethyl-2-(1-phenyl-1H-pyrazol-4-yl)- | 382 | 382 | HCl | 2 |
| | N-(piperidin-3-yl)-1,3-thiazole-4- | | | Salt | |
| | carboxamide | | | | |
| 152 | methyl({1-[2-(1-phenyl-1H-pyrazol-4- | 367 | 368 | Free | 2 |
| | yl)-1,3-thiazole-4-carbonyl]pyrrolidin- | | | Base | |
| | 2-yl}methyl)amine | | | | |
| 153 | 1-{3-methyl-1-[2-(1-phenyl-1H- | 382 | 382 | HCl | 2 |
| | pyrazol-4-yl)-1,3-thiazole-4- | | | Salt | |
| | carbonyl]piperidin-2-yl}methanamine | | | | |
| 154 | 1-[2-(1-phenyl-1H-pyrazol-4-yl)-1,3- | 353 | 354 | HCl | 2 |
| | thiazole-4-carbonyl]-1,4-diazepane | | | Salt | |
| 156 | 1-[(2R)-1-[2-(1-phenyl-1H-pyrazol-4- | 353 | 354 | HCl | see full |
| | yl)-1,3-thiazole-4-carbonyl]pyrrolidin- | | | Salt | experimental |
| | 2-yl]methanamine | | | | |
| 158 | {1-[2-(1-phenyl-1H-pyrazol-4-yl)-1,3- | 369 | 370 | Free | 2 |
| | thiazole-4-carbonyl]piperazin-2- | | | Base | |
| | yl}methanol | | | | |
| 159 | N-(3-aminopropyl)-2-(1-phenyl- | 327 | 328 | HCl | 2 |
| | 1H-pyrazol-4-yl)-1,3-thiazole-4- | | | Salt | |
| | carboxamide | | | | |
| 160 | N-(3-methylpiperidin-3-yl)-2-(1- | 367 | 368 | HCl | 2 |
| | phenyl-1H-pyrazol-4-yl)-1,3- | | | Salt | |
| | thiazole-4-carboxamide | | | | |
| 161 | N-(5-aminopentyl)-2-(1-phenyl- | 355 | 356 | Free | 2 |
| | 1H-pyrazol-4-yl)-1,3-thiazole-4- | | | Base | |
| | carboxamide | | | | |
| 162 | N-(2-aminoethyl)-2-(1-phenyl-1H- | 313 | 314 | HCl | 2 |
| | pyrazol-4-yl)-1,3-thiazole-4- | | | Salt | |
| | carboxamide | | | | |
| 163 | 2-(1-phenyl-1H-pyrazol-4-yl)-N- | 396 | 396 | HCl | see full |
| | (piperidin-4-yl)-N-(propan-2-yl)-1,3- | | | Salt | experimental |
| | thiazole-4-carboxamide | | | | |
| 165 | 1-{1-[2-(1-phenyl-1H-pyrazol-4-yl)- | 382 | 382 | HCl | 2 |
| | 1,3-thiazole-4-carbonyl]piperidin-4- | | | Salt | |
| | yl}ethan-1-amine | | | | |
| 167 | 1-[2-(1-phenyl-1H-pyrazol-4-yl)-1,3- | 353 | 354 | HCl | 2 |
| | thiazole-4- | | | Salt | |
| | carbonyl]piperidin-3-amine | | | | |
| 170 | 2-(1-phenyl-1H-pyrazol-4-yl)-N- | 381 | 382 | HCl | see full |
| | propyl-N-[(3S)-pyrrolidin-3-yl]-1,3- | | | Salt | experimental |
| | thiazole-4-carboxamide | | | | |
| 171 | 2-(1-phenyl-1H-pyrazol-4-yl)-N- | 381 | 382 | HCl | see full |
| | propyl-N-[(3R)-pyrrolidin-3-yl]-1,3- | | | Salt | experimental |
| | thiazole-4-carboxamide | | | | |
| 175 | N-[2-(2-aminoethoxy)ethyl]-2-(1 | 357 | 358 | HCl | 2 |
| | phenyl-1H-pyrazol-4-yl)-1,3-thiazole- | | | Salt | |
| | 4-carboxamide | | | | |
| 177 | 2-(1-phenyl-1H-pyrazol-4-yl)-N- | 367 | 368 | HCl | 2 |
| | [(piperidin-4-yl)methyl]-1,3-thiazole- | | | Salt | |
| | 4-carboxamide | | | | |
| 178 | N-[(morpholin-2-yl)methyl]-2-(1 | 369 | 370 | HCl | 2 |
| | phenyl-1H-pyrazol-4-yl)-1,3- | | | Salt | |
| | thiazole-4-carboxamide | | | | |
| 179 | 2-(1-phenyl-1H-pyrazol-4-yl)-N- | 353 | 354 | HCl | 2 |
| | [(pyrrolidin-3-yl)methyl]-1,3- | | | Salt | |
| | thiazole-4-carboxamide | | | | |
| 180 | N-(azetidin-3-yl)-2-(1-phenyl- | 325 | 326 | HCl | 2 |
| | 1H-pyrazol-4-yl)-1,3-thiazole-4- | | | Salt | |
| | carboxamide | | | | |
| 181 | methyl 4-[2-(1-phenyl-1H-pyrazol-4- | 411 | 412 | HCl | 2 |
| | yl)-1,3-thiazole-4- | | | Salt | |
| | amido]piperidine-4-carboxylate | | | | |
| 183 | 2-(1-phenyl-1H-pyrazol-4-yl)-N- | 339 | 340 | HCl | 2 |
| | [(3R)-pyrrolidin-3-yl]-1,3-thiazole-4- | | | Salt | |
| | carboxamide | | | | |
| 184 | 2-(1-phenyl-1H-pyrazol-4-yl)-N- | 339 | 340 | HCl | see full |
| | (pyrrolidin-3-yl)-1,3-thiazole-4- | | | Salt | experimental |
| | carboxamide | | | | |
| 305 | methyl({1-[2-(1-phenyl-1H- | 367.47 | 368.2 | Free Base | 1 |
| | pyrazol-4-yl)-1,3-thiazole-4- | | | | |
| | carbonyl]pyrrolidin-3- | | | | |
| | yl}methyl)amine | | | | |
| 306 | 2-[1-(3-fluorophenyl)-1H- | 413.17 | 414.2 | HCl salt | 3 |
| | pyrazol-4-yl]-N-(piperidin-4- | | | | |
| | yl)-N-(propan-2-yl)-1,3- | | | | |
| | thiazole-4-carboxamide | | | | |
| 307 | N-(piperidin-4-yl)-N-(propan-2- | 396.17 | 397.2 | HCl salt | 3 |
| | yl)-2-[1-(pyridin-2-yl)-1H- | | | | |
| | pyrazol-4-yl]-1,3-thiazole-4- | | | | |
| | carboxamide | | | | |
| 308 | N-propyl-2-[1-(pyrimidin-5-yl)- | 383.47 | 384.2 | HCl salt | 3 |
| | 1H-pyrazol-4-yl]-N-[(3R)- | | | | |
| | pyrrolidin-3-yl]-1,3-thiazole-4- | | | | |
| | carboxamide | | | | |
| 309 | N-propyl-2-[1-(pyridazin-3-yl)- | 383.47 | 384 | HCl Salt | 3 |
| | 1H-pyrazol-4-yl]-N-[(3R)- | | | | |
| | pyrrolidin-3-yl]-1,3-thiazole-4- | | | | |
| | carboxamide | | | | |
| 310 | 5-[1-(4-fluorophenyl)-1H- | 395.48 | 396.2 | HCl salt | 2 |
| | pyrazol-4-yl]-N-(piperidin-4- | | | | |
| | yl)-N-(propan-2-yl)-1H-pyrrole- | | | | |
| | 2-carboxamide | | | | |
| 311 | 2-(1-phenyl-1H-pyrazol-4-yl)- | 381.16 | 382.1 | HCl salt | 1 |
| | N-(propan-2-yl)-N-[(3S)- | | | | |
| | pyrrolidin-3-yl]-1,3-thiazole-4- | | | | |
| | carboxamide | | | | |
| 312 | 2-(1-phenyl-1H-pyrazol-4-yl)- | 381.16 | 382.2 | HCl salt | 1 |
| | N-(propan-2-yl)-N-[(3R)- | | | | |
| | pyrrolidin-3-yl]-1,3-thiazole-4- | | | | |
| | carboxamide | | | | |
| 313 | 5-(1-phenyl-1H-pyrazol-4-yl)- | 363.21 | 364.1 | HCl salt | 2 |
| | N-(propan-2-yl)-N-[(3R)- | | | | |
| | pyrrolidin-3-yl]-1H-pyrrole-2- | | | | |
| | carboxamide | | | | |
| 314 | 5-(1-phenyl-1H-pyrazol-4-yl)- | 363.47 | 364.1 | HCl Salt | 2 |
| | N-(propan-2-yl)-N-[(3S)- | | | | |
| | pyrrolidin-3-yl]-1H-pyrrole-2- | | | | |
| | carboxamide | | | | |
| 315 | N-[(1S,4S)-2- | 407.54 | 408.2 | HCl Salt | 1 |
| | azabicyclo[2.2.1]heptan-5-yl]- | | | | |
| | 2-(1-phenyl-1H-pyrazol-4-yl)- | | | | |
| | N-(propan-2-yl)-1,3-thiazole-4- | | | | |
| | carboxamide | | | | |
| 316 | N-[(1R,4R)-2- | 407.54 | 408 | HCl salt | 1 |
| | azabicyclo[2.2.1]heptan-5-yl]- | | | | |
| | 2-(1-phenyl-1H-pyrazol-4-yl)- | | | | |
| | N-(propan-2-yl)-1,3-thiazole-4- | | | | |
| | carboxamide | | | | |
| 317 | N-{3-azabicyclo[3.1.1]heptan- | 407.18 | 408.2 | HCl salt | 1 |
| | 6-yl}-2-(1-phenyl-1H-pyrazol-4- | | | | |
| | yl)-N-(propan-2-yl)-1,3- | | | | |
| | thiazole-4-carboxamide | | | | |
| 318 | N-[(1R,5S,6S)-3- | 393.51 | 394.1 | HCl salt | 1 |
| | azabicyclo[3.1.0]hexan-6-yl]-2- | | | | |
| | (1-phenyl-1H-pyrazol-4-yl)-N- | | | | |
| | (propan-2-yl)-1,3-thiazole-4- | | | | |
| | carboxamide | | | | |
| 319 | N-{2-azaspiro[3.3]heptan-6-yl}- | 407.54 | 408.1 | Methane | 1 |
| | 2-(1-phenyl-1H-pyrazol-4-yl)- | | | sulfonate | |
| | N-(propan-2-yl)-1,3-thiazole-4- | | | | |
| | carboxamide | | | | |
| 320 | N-[(3aR,6aS)- | 421.56 | 422.1 | HCl salt | 1 |
| | octahydrocyclopenta[c]pyrrol- | | | | |
| | 5-yl]-2-(1-phenyl-1H-pyrazol-4- | | | | |
| | yl)-N-(propan-2-yl)-1,3- | | | | |
| | thiazole-4-carboxamide | | | | |
| 321 | N-(piperidin-4-yl)-N-(propan-2- | 396.51 | 397 | Free base | 3 |
| | yl)-2-[1-(pyridin-3-yl)-1H- | | | | |
| | pyrazol-4-yl]-1,3-thiazole-4- | | | | |
| | carboxamide | | | | |
| 322 | N-(piperidin-4-yl)-N-(propan-2- | 396.51 | 397 | HCl salt | 3 |
| | yl)-2-[1-(pyridin-4-yl)-1H- | | | | |
| | pyrazol-4-yl]-1,3-thiazole-4- | | | | |
| | carboxamide | | | | |
| 323 | N-(piperidin-4-yl)-N-(propan-2- | 397.5 | 398.1 | Free base | 3 |
| | yl)-2-[1-(pyridazin-3-yl)-1H- | | | | |
| | pyrazol-4-yl]-1,3-thiazole-4- | | | | |
| | carboxamide | | | | |
| 324 | N-(piperidin-4-yl)-N-(propan-2- | 397.5 | 398.1 | HCl salt | 3 |
| | yl)-2-[1-(pyrazin-2-yl)-1H- | | | | |
| | pyrazol-4-yl]-1,3-thiazole-4- | | | | |
| | carboxamide | | | | |
| 325 | (1S,4S)-2-[2-(1-phenyl-1H- | 351.43 | 351.8 | HCl salt | 1 |
| | pyrazol-4-yl)-1,3-thiazole-4- | | | | |
| | carbonyl]-2,5- | | | | |
| | diazabicyclo[2.2.1]heptane | | | | |
| 326 | (1R,4R)-2-[2-(1-phenyl-1H- | 351.43 | 351.8 | HCl Salt | 1 |
| | pyrazol-4-yl)-1,3-thiazole-4- | | | | |
| | carbonyl]-2,5- | | | | |
| | diazabicyclo[2.2.1]heptane | | | | |
| 327 | N-[(1S,4S)-2- | 365.45 | 365.9 | HCl salt | 1 |
| | azabicyclo[2.2.1]heptan-5-yl]- | | | | |
| | 2-(1-phenyl-1H-pyrazol-4-yl)- | | | | |
| | 1,3-thiazole-4-carboxamide | | | | |
| 328 | N-(propan-2-yl)-2-[1- | 383.47 | 384.1 | HCl salt | 3 |
| | (pyrimidin-2-yl)-1H-pyrazol-4- | | | | |
| | yl]-N-[(3R)-pyrrolidin-3-yl]-1,3- | | | | |
| | thiazole-4-carboxamide | | | | |
| 329 | N-(piperidin-4-yl)-N-(propan-2- | 397.5 | 398 | HCl salt | 3 |
| | yl)-2-[1-(pyrimidin-2-yl)-1H- | | | | |
| | pyrazol-4-yl]-1,3-thiazole-4- | | | | |
| | carboxamide | | | | |
| 330 | N-(propan-2-yl)-2-[1- | 383.47 | 384.2 | HCl Salt | 3 |
| | (pyrimidin-5-yl)-1H-pyrazol-4- | | | | |
| | yl]-N-[(3R)-pyrrolidin-3-yl]-1,3- | | | | |
| | thiazole-4-carboxamide | | | | |
| 331 | N-(piperidin-4-yl)-N-(propan-2- | 397.5 | 398.1 | HCl Salt | 3 |
| | yl)-2-[1-(pyrimidin-5-yl)-1H- | | | | |
| | pyrazol-4-yl]-1,3-thiazole-4- | | | | |
| | carboxamide | | | | |
| 332 | N-(propan-2-yl)-2-[1- | 383.47 | 384.1 | Free base | 3 |
| | (pyridazin-3-yl)-1H-pyrazol-4- | | | | |
| | yl]-N-[(3R)-pyrrolidin-3-yl]-1,3- | | | | |
| | thiazole-4-carboxamide | | | | |
| 333 | N-(propan-2-yl)-2-[1-(pyrazin- | 383.47 | 384.1 | HCl salt | 3 |
| | 2-yl)-1H-pyrazol-4-yl]-N-[(3R)- | | | | |
| | pyrrolidin-3-yl]-1,3-thiazole-4- | | | | |
| | carboxamide | | | | |
| 334 | 4-(1-phenyl-1H-pyrazol-4-yl)- | 363.47 | 364.1 | HCl salt | 2 |
| | N-(propan-2-yl)-N-[(3R)- | | | | |
| | pyrrolidin-3-yl]-1H-pyrrole-2- | | | | |
| | carboxamide | | | | |
| 335 | 2-(1-phenyl-1H-1,2,4-triazol-3- | 396.51 | 397.1 | HCl salt | 2 |
| | yl)-N-(piperidin-4-yl)-N- | | | | |
| | (propan-2-yl)-1,3-thiazole-4- | | | | |
| | carboxamide | | | | |
| 336 | 2-(1-phenyl-1H-pyrazol-4-yl)- | 379.46 | 380.4 | HCl salt | 2 |
| | N-(piperidin-4-yl)-N-(propan-2- | | | | |
| | yl)-1,3-oxazole-4-carboxamide | | | | |
| 337 | 2-[1-(4-cyclopropylphenyl)-1H- | 435.59 | 436.1 | HCl salt | See |
| | pyrazol-4-yl]-N-(piperidin-4- | | | | Experimental |
| | yl)-N-(propan-2-yl)-1,3- | | | | details |
| | thiazole-4-carboxamide | | | | |
| 338 | 2-[1-(4-tert-butylphenyl)-1H- | 451.63 | 452 | HCl Salt | 3 |
| | pyrazol-4-yl]-N-(piperidin-4- | | | | |
| | yl)-N-(propan-2-yl)-1,3- | | | | |
| | thiazole-4-carboxamide | | | | |
| 339 | N-(piperidin-4-yl)-N-(propan-2- | 437.61 | 438 | HCl salt | 3 |
| | yl)-2-{1-[4-(propan-2- | | | | |
| | yl)phenyl]-1H-pyrazol-4-yl}- | | | | |
| | 1,3-thiazole-4-carboxamide | | | | |
| 340 | 2-(1-phenyl-1H-pyrazol-4-yl)- | 378.48 | 379 | HCl Salt | 2 |
| | N-(piperidin-4-yl)-N-(propan-2- | | | | |
| | yl)-1H-imidazole-4- | | | | |
| | carboxamide | | | | |
| 341 | 5-(1-phenyl-1H-pyrazol-4-yl)- | 378.48 | 379 | HCl Salt | 2 |
| | N-(piperidin-4-yl)-N-(propan-2- | | | | |
| | yl)-1H-imidazole-2- | | | | |
| | carboxamide | | | | |
| 342 | N-ethyl-2-(1-phenyl-1H- | 381.5 | 382 | HCl salt | See Experimental |
| | pyrazol-4-yl)-N-[(3S)-piperidin- | | | | details |
| | 3-yl]-1,3-thiazole-4- | | | | |
| | carboxamide | | | | |
| 343 | N-ethyl-2-(1-phenyl-1H- | 381.5 | 382.1 | Free base | See Experimental |
| | pyrazol-4-yl)-N-[(3R)-piperidin- | | | | details |
| | 3-yl]-1,3-thiazole-4- | | | | |
| | carboxamide | | | | |
| 344 | 5-(1-phenyl-1H-pyrazol-4-yl)- | 364.45 | 365.3 | HCl salt | 2 |
| | N-(propan-2-yl)-N-[(3S)- | | | | |
| | pyrrolidin-3-yl]-1H-imidazole- | | | | |
| | 2-carboxamide | | | | |
| 345 | 5-(1-phenyl-1H-pyrazol-4-yl)- | 364.45 | 365 | HCl salt | 2 |
| | N-(propan-2-yl)-N-[(3R)- | | | | |
| | pyrrolidin-3-yl]-1H-imidazole- | | | | |
| | 2-carboxamide | | | | |
| 346 | N-(propan-2-yl)-2-[1-(pyridin- | 382.4 | 383.2 | HCl salt | 3 |
| | 2-yl)-1H-pyrazol-4-yl]-N-[(3S)- | 9 | | | |
| | pyrrolidin-3-yl]-1,3-thiazole-4- | | | | |
| | carboxamide | | | | |
| 347 | N-methyl-2-(1-phenyl-1H- | 367.47 | 368.1 | HCl salt | 1 |
| | pyrazol-4-yl)-N-(piperidin-4- | | | | |
| | yl)-1,3-thiazole-4-carboxamide | | | | |
| 348 | N-methyl-2-(1-phenyl-1H- | 351.41 | 352.2 | HCl salt | 2 |
| | pyrazol-4-yl)-N-(piperidin-4- | | | | |
| | yl)-1,3-oxazole-4-carboxamide | | | | |
| 349 | N-ethyl-2-(1-phenyl-1H- | 365.44 | 366.2 | HCl salt | 2 |
| | pyrazol-4-yl)-N-(piperidin-4- | | | | |
| | yl)-1,3-oxazole-4-carboxamide | | | | |
| 350 | N-methyl-2-(1-phenyl-1H- | 350.4 | 350.9 | HCl salt | 2 |
| | pyrazol-4-yl)-N-(piperidin-4- | 3 | | | |
| | yl)-1H-imidazole-4- | | | | |
| | carboxamide | | | | |
| 351 | N-ethyl-2-(1-phenyl-1H- | 364.45 | 365 | HCl salt | 2 |
| | pyrazol-4-yl)-N-(piperidin-4- | | | | |
| | yl)-1H-imidazole-4- | | | | |
| | carboxamide | | | | |
| 352 | N-methyl-5-(1-phenyl-1H- | 350.43 | 351 | HCl salt | 2 |
| | pyrazol-4-yl)-N-(piperidin-4- | | | | |
| | yl)-1H-imidazole-2- | | | | |
| | carboxamide | | | | |
| 353 | N-ethyl-5-(1-phenyl-1H- | 364.45 | 365 | HCl salt | 2 |
| | pyrazol-4-yl)-N-(piperidin-4- | | | | |
| | yl)-1H-imidazole-2- | | | | |
| | carboxamide | | | | |
| 354 | N-methyl-N-(piperidin-4-yl)-2- | 368.46 | 369.1 | HCl salt | 3 |
| | [1-(pyridin-2-yl)-1H-pyrazol-4- | | | | |
| | yl]-1,3-thiazole-4-carboxamide | | | | |
| 355 | N-ethyl-N-(piperidin-4-yl)-2-[1- | 382.49 | 383.1 | HCl salt | 3 |
| | (pyridin-2-yl)-1H-pyrazol-4-yl]- | | | | |
| | 1,3-thiazole-4-carboxamide | | | | |
| 356 | N-methyl-2-(1-phenyl-1H- | 353.44 | 354.1 | HCl salt | 1 |
| | pyrazol-4-yl)-N-[(3S)- | | | | |
| | pyrrolidin-3-yl]-1,3-thiazole-4- | | | | |
| | carboxamide | | | | |
| 357 | N-ethyl-2-(1-phenyl-1H- | 367.47 | 368.2 | HCl salt | 1 |
| | pyrazol-4-yl)-N-[(3S)- | | | | |
| | pyrrolidin-3-yl]-1,3-thiazole-4- | | | | |
| | carboxamide | | | | |
| 358 | N-methyl-2-(1-phenyl-1H- | 336.4 | 336.9 | HCl salt | 2 |
| | pyrazol-4-yl)-N-[(3S)- | | | | |
| | pyrrolidin-3-yl]-1H-imidazole- | | | | |
| | 4-carboxamide | | | | |
| 359 | N-methyl-5-(1-phenyl-1H- | 336.4 | 336.9 | HCl salt | 2 |
| | pyrazol-4-yl)-N-[(3S)- | | | | |
| | pyrrolidin-3-yl]-1H-imidazole- | | | | |
| | 2-carboxamide | | | | |
| 360 | N-methyl-2-(1-phenyl-1H- | 337.15 | 338 | HCl salt | 2 |
| | pyrazol-4-yl)-N-[(3S)- | | | | |
| | pyrrolidin-3-yl]-1,3-oxazole-4- | | | | |
| | carboxamide | | | | |
| 361 | N-ethyl-2-(1-phenyl-1H- | 351.41 | 351.8 | HCl salt | 2 |
| | pyrazol-4-yl)-N-[(3S)- | | | | |
| | pyrrolidin-3-yl]-1,3-oxazole-4- | | | | |
| | carboxamide | | | | |
| 362 | N-methyl-2-(2-phenyl-2H- | 354.43 | 355.1 | HCl salt | 4 |
| | 1,2,3-triazol-4-yl)-N-[(3S)- | | | | |
| | pyrrolidin-3-yl]-1,3-thiazole-4- | | | | |
| | carboxamide | | | | |
| 363 | N-methyl-2-(2-phenyl-2H- | 354.43 | 355.1 | HCl salt | 4 |
| | 1,2,3-triazol-4-yl)-N-[(3R)- | | | | |
| | pyrrolidin-3-yl]-1,3-thiazole-4- | | | | |
| | carboxamide | | | | |
| 364 | N-ethyl-2-(2-phenyl-2H-1,2,3- | 368.46 | 369.1 | HCl salt | 4 |
| | triazol-4-yl)-N-[(3S)-pyrrolidin- | | | | |
| | 3-yl]-1,3-thiazole-4- | | | | |
| | carboxamide | | | | |
| 365 | N-ethyl-2-(2-phenyl-2H-1,2,3- | 368.46 | 369 | HCl salt | 4 |
| | triazol-4-yl)-N-[(3R)-pyrrolidin- | | | | |
| | 3-yl]-1,3-thiazole-4- | | | | |
| | carboxamide | | | | |
| 366 | N-methyl-2-[1-(pyridin-2-yl)- | 354.43 | 355.1 | HCl salt | 3 |
| | 1H-pyrazol-4-yl]-N-[(3S)- | | | | |
| | pyrrolidin-3-yl]-1,3-thiazole-4- | | | | |
| | carboxamide | | | | |
| 367 | N-ethyl-2-[1-(pyridin-2-yl)-1H- | 368.46 | 369.1 | HCl salt | 3 |
| | pyrazol-4-yl]-N-[(3S)- | | | | |
| | pyrrolidin-3-yl]-1,3-thiazole-4- | | | | |
| | carboxamide | | | | |
| 368 | N-methyl-N-(piperidin-4-yl)-2- | 351.41 | 351.9 | HCl salt | 2 |
| | [1-(pyridin-2-yl)-1H-pyrazol-4- | | | | |
| | yl]-1H-imidazole-4- | | | | |
| | carboxamide | | | | |
| 369 | N-ethyl-N-(piperidin-4-yl)-2-[1- | 365.44 | 366.3 | HCl Salt | 2 |
| | (pyridin-2-yl)-1H-pyrazol-4-yl]- | | | | |
| | 1H-imidazole-4-carboxamide | | | | |
| 370 | N-methyl-N-(piperidin-4-yl)-2- | 352.4 | 353 | HCl salt | 5 |
| | [1-(pyridin-2-yl)-1H-pyrazol-4- | | | | |
| | yl]-1,3-oxazole-4-carboxamide | | | | |
| 371 | N-ethyl-N-(piperidin-4-yl)-2-[1- | 366.43 | 367 | HCl salt | 5 |
| | (pyridin-2-yl)-1H-pyrazol-4-yl]- | | | | |
| | 1,3-oxazole-4-carboxamide | | | | |
| 372 | 2-(1-phenyl-1H-pyrazol-4-yl)- | 353.4 | 354 | HCl salt | 1 |
| | N-(piperidin-4-yl)-1,3-thiazole- | 4 | | | |
| | 4-carboxamide | | | | |
| 373 | N-(piperidin-4-yl)-2-[1- | 354.43 | 355.1 | HCl salt | 3 |
| | (pyridin-2-yl)-1H-pyrazol-4-yl]- | | | | |
| | 1,3-thiazole-4-carboxamide | | | | |
| 374 | N-(piperidin-4-yl)-N-(propan-2- | 379.47 | 379.9 | HCl salt | 2 |
| | yl)-2-[1-(pyridin-2-yl)-1H- | | | | |
| | pyrazol-4-yl]-1H-imidazole-4- | | | | |
| | carboxamide | | | | |
| 375 | N-(piperidin-4-yl)-N-(propan-2- | 380.45 | 381 | HCl salt | 2 |
| | yl)-2-[1-(pyridin-2-yl)-1H- | | | | |
| | pyrazol-4-yl]-1,3-oxazole-4- | | | | |
| | carboxamide | | | | |
| 376 | (1R,4R)-2-(2-{1-[2- | 435.1 | 436 | HCl Salt | 3 |
| | (trifluoromethoxy)phenyl]-1H- | | | | |
| | pyrazol-4-yl}-1,3-thiazole-4- | | | | |
| | carbonyl)-2,5- | | | | |
| | diazabicyclo[2.2.1]heptane | | | | |
| 377 | N-(piperidin-4-yl)-N-(propan-2- | 479.52 | 480 | HCl Salt | 3 |
| | yl)-2-{1-[2- | | | | |
| | (trifluoromethoxy)phenyl]-1H- | | | | |
| | pyrazol-4-yl}-1,3-thiazole-4- | | | | |
| | carboxamide | | | | |
| 378 | (1R,4R)-2-(2-{1-[2- | 419.36 | 420 | HCl salt | 5 |
| | (trifluoromethoxy)phenyl]-1H- | | | | |
| | pyrazol-4-yl}-1,3-oxazole-4- | | | | |
| | carbonyl)-2,5- | | | | |
| | diazabicyclo[2.2.1]heptane | | | | |
| 379 | N-(piperidin-4-yl)-N-(propan-2- | 463.46 | 464 | HCl salt | 5 |
| | yl)-2-{1-[2- | | | | |
| | (trifluoromethoxy)phenyl]-1H- | | | | |
| | pyrazol-4-yl}-1,3-oxazole-4- | | | | |
| | carboxamide | | | | |
| 380 | 2-(1-phenyl-1H-1,2,4-triazol-3- | 382.49 | 382.9 | HCl Salt | 2 |
| | yl)-N-(propan-2-yl)-N-[(3S)- | | | | |
| | pyrrolidin-3-yl]-1,3-thiazole-4- | | | | |
| | carboxamide | | | | |
| 381 | 2-(1-phenyl-1H-pyrazol-4-yl)- | 365.44 | 366.1 | HCl salt | 2 |
| | N-(propan-2-yl)-N-[(3S)- | | | | |
| | pyrrolidin-3-yl]-1,3-oxazole-4- | | | | |
| | carboxamide | | | | |
| 382 | N-(3,3-difluoropiperidin-4-yl)- | 431.51 | 432 | HCl salt | 1 |
| | 2-(1-phenyl-1H-pyrazol-4-yl)- | | | | |
| | N-(propan-2-yl)-1,3-thiazole-4- | | | | |
| | carboxamide | | | | |
| 383 | N-(3-fluoropiperidin-4-yl)-2-(1- | 413.52 | 414.1 | HCl salt | 1 |
| | phenyl-1H-pyrazol-4-yl)-N- | | | | |
| | (propan-2-yl)-1,3-thiazole-4- | | | | |
| | carboxamide | | | | |
| 384 | 4-{octahydropyrrolo[2,3- | 365.45 | 366 | HCl salt | 1 |
| | c]pyrrole-1-carbonyl}-2-(1- | | | | |
| | phenyl-1H-pyrazol-4-yl)-1,3- | | | | |
| | thiazole | | | | |
| 385 | 4-{octahydropyrrolo[3,4- | 381.45 | 382 | HCl salt | 1 |
| | b][1,4]oxazine-4-carbonyl}-2- | | | | |
| | (1-phenyl-1H-pyrazol-4-yl)-1,3- | | | | |
| | thiazole | | | | |
| 386 | 2-(2-phenyl-2H-1,2,3-triazol-4- | 396.51 | 396.9 | HCl salt | 4 |
| | yl)-N-(piperidin-4-yl)-N- | | | | |
| | (propan-2-yl)-1,3-thiazole-4- | | | | |
| | carboxamide | | | | |
| 387 | 2-[2-(1-phenyl-1H-pyrazol-4- | 351.43 | 352 | HCl salt | 1 |
| | yl)-1,3-thiazole-4-carbonyl]- | | | | |
| | 2,6-diazaspiro[3.3]heptane | | | | |
| 388 | 2-(1-phenyl-1H-pyrazol-4-yl)-4- | 363.44 | 364 | HCl salt | 1 |
| | {1H,2H,3H,4H,5H,6H- | | | | |
| | pyrrolo[3,4-c]pyrrole-2- | | | | |
| | carbonyl}-1,3-thiazole | | | | |
| 389 | 2-(1-methyl-1H-pyrazol-4-yl)- | 319.43 | 320 | HCl salt | 6 |
| | N-(propan-2-yl)-N-[(3S)- | | | | |
| | pyrrolidin-3-yl]-1,3-thiazole-4- | | | | |
| | carboxamide | | | | |
| 390 | N-(propan-2-yl)-2-[1-(propan- | 347.48 | 348 | HCl salt | 6 |
| | 2-yl)-1H-pyrazol-4-yl]-N-[(3S)- | | | | |
| | pyrrolidin-3-yl]-1,3-thiazole-4- | | | | |
| | carboxamide | | | | |
| 391 | 2-(1-tert-butyl-1H-pyrazol-4- | 361.51 | 362.1 | HCl salt | 3 |
| | yl)-N-(propan-2-yl)-N-[(3S)- | | | | |
| | pyrrolidin-3-yl]-1,3-thiazole-4- | | | | |
| | carboxamide | | | | |
| 392 | 2-(1-methyl-1H-pyrazol-4-yl)- | 333.45 | 334 | HCl salt | 6 |
| | N-(piperidin-4-yl)-N-(propan-2- | | | | |
| | yl)-1,3-thiazole-4-carboxamide | | | | |
| 393 | N-(piperidin-4-yl)-N-(propan-2- | 361.51 | 362.1 | HCl salt | 6 |
| | yl)-2-[1-(propan-2-yl)-1H- | | | | |
| | pyrazol-4-yl]-1,3-thiazole-4- | | | | |
| | carboxamide | | | | |
| 394 | 2-(1-tert-butyl-1H-pyrazol-4- | 375.54 | 376.1 | HCl salt | 3 |
| | yl)-N-(piperidin-4-yl)-N- | | | | |
| | (propan-2-yl)-1,3-thiazole-4- | | | | |
| | carboxamide | | | | |
| 395 | 2-[1-(2-chlorophenyl)-1H- | 415.94 | 416 | HCl Salt | 3 |
| | pyrazol-4-yl]-N-(propan-2-yl)- | | | | |
| | N-[(3S)-pyrrolidin-3-yl]-1,3- | | | | |
| | thiazole-4-carboxamide | | | | |
| 396 | N-(propan-2-yl)-N-[(3S)- | 465.5 | 466 | HCl Salt | 3 |
| | pyrrolidin-3-yl]-2-{1-[2- | | | | |
| | (trifluoromethoxy)phenyl]-1H- | | | | |
| | pyrazol-4-yl}-1,3-thiazole-4- | | | | |
| | carboxamide | | | | |
| 397 | 2-[1-(2-methylphenyl)-1H- | 395.53 | 396.1 | HCl Salt | 3 |
| | pyrazol-4-yl]-N-(propan-2-yl)- | | | | |
| | N-[(3S)-pyrrolidin-3-yl]-1,3- | | | | |
| | thiazole-4-carboxamide | | | | |
| 398 | 2-[1-(2-methoxyphenyl)-1H- | 411.52 | 412.1 | HCl Salt | 3 |
| | pyrazol-4-yl]-N-(propan-2-yl)- | | | | |
| | N-[(3S)-pyrrolidin-3-yl]-1,3- | | | | |
| | thiazole-4-carboxamide | | | | |

TABLE 2
Compounds according to the present invention, chemical structures, and names

| Compound/ | | |
|---|---|---|
| Formula Number | Structure | Chemical Name |
| 5 | 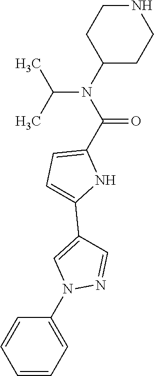 [SMILES: CC(C)N(C(=O)c1ccc(-c2cnn(-c3ccccc3)c2)[nH]1)C1CCNCC1] | 5-(1-phenyl-1H-pyrazol-4- yl)-N-(piperidin-4-yl)-N- (propan-2-yl)-1H-pyrrole-2- carboxamide |
| 6 | 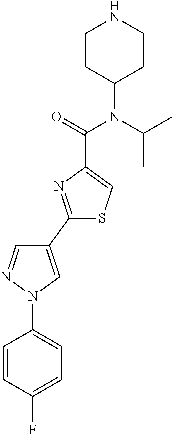 | 2-[1-(4-fluorophenyl)-1H- pyrazol-4-yl]-N-(piperidin- 4-yl)-N-(propan-2-yl)-1,3- thiazole-4-carboxamide |
| 8 | 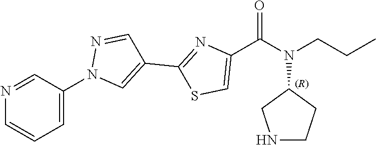 | N-propyl-2-[1-(pyridin-3- yl)-1H-pyrazol-4-yl]-N- [(3R)-pyrrolidin-3-yl]-1,3- thiazole-4-carboxamide |
| 9 | 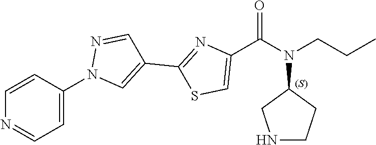 | N-propyl-2-[1-(pyridin-4- yl)-1H-pyrazol-4-yl]-N- [(3S)-pyrrolidin-3-yl]-1,3- thiazole-4-carboxamide |
| 11 | 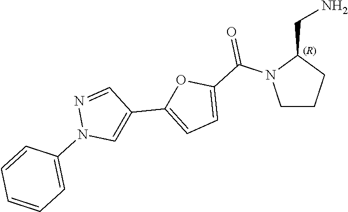 | 1-[(2R)-1-[5-(1-phenyl-1H- pyrazol-4-yl)furan-2- carbonyl]pyrrolidin-2- yl]methanamine |
| 12 | 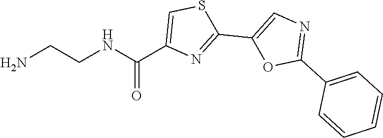 [SMILES: NCCNC(=O)c1csc(-c2cnc(-c3ccccc3)o2)n1] | N-(2-aminoethyl)-2-(2- phenyl-1,3-oxazol-5-yl)- 1,3-thiazole-4-carboxamide |
| 14 | 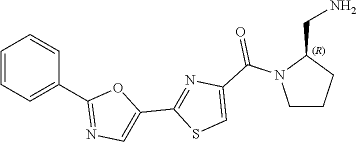 | 1-[(2R)-1-[2-(2-phenyl-1,3- oxazol-5-yl)-1,3-thiazole-4- carbonyl]pyrrolidin-2- yl]methanamine |
| 16 | 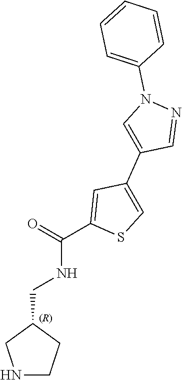 | 4-(1-phenyl-1H-pyrazol-4- yl)-N-{[(3R)-pyrrolidin-3- yl]methyl}thiophene-2- carboxamide |
| 17 | 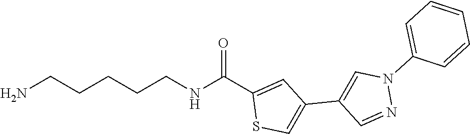 [SMILES: NCCCCCNC(=O)c1cc(-c2cnn(-c3ccccc3)c2)cs1] | N-(5-aminopentyl)-4-(1- phenyl-1H-pyrazol-4- yl)thiophene-2- carboxamide |
| 18 | 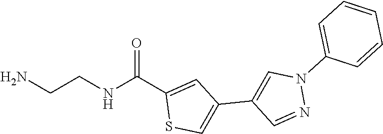 [SMILES: NCCNC(=O)c1cc(-c2cnn(-c3ccccc3)c2)cs1] | N-(2-aminoethyl)-4-(1- phenyl-1H-pyrazol-4- yl)thiophene-2- carboxamide |
| 21 | 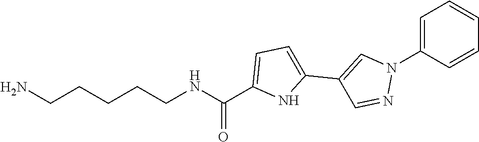 [SMILES: NCCCCCNC(=O)c1ccc(-c2cnn(-c3ccccc3)c2)[nH]1] | N-(5-aminopentyl)-5-(1- phenyl-1H-pyrazol-4-yl)- 1H-pyrrole-2-carboxamide |
| 22 | 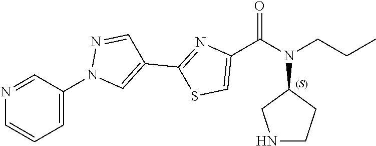 | N-propyl-2-[1-(pyridin-3- yl)-1H-pyrazol-4-yl]-N- [(3S)-pyrrolidin-3-yl]-1,3- thiazole-4-carboxamide |
| 23 | 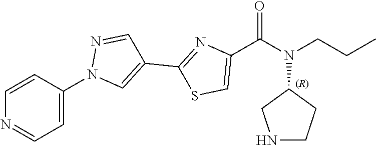 | N-propyl-2-[1-(pyridin-4- yl)-1H-pyrazol-4-yl]-N- [(3S)-pyrrolidin-3-yl]-1,3- thiazole-4-carboxamide |
| 25 | 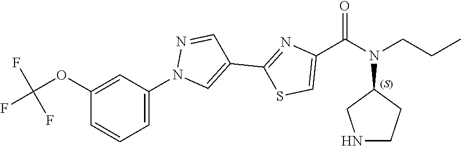 | N-propyl-N-[(3S)- pyrrolidin-3-yl]-2-{1-[3- (trifluoromethoxy)phenyl]- 1H-pyrazol-4-yl}-1,3- thiazole-4-carboxamide |
| 26 | 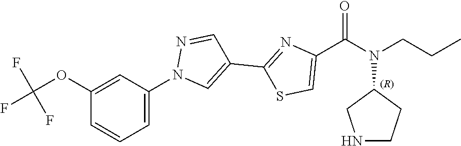 | N-propyl-N-[(3R)- pyrrolidin-3-yl]-2-{1-[3- (trifluoromethoxy)phenyl]- 1H-pyrazol-4-yl}-1,3- thiazole-4-carboxamide |
| 28 | 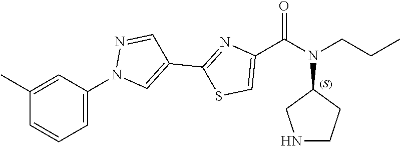 | 2-[1-(3-methylphenyl)-1H- pyrazol-4-yl]-N-propyl-N- [(3S)-pyrrolidin-3-yl]-1,3- thiazole-4-carboxamide |
| 29 | 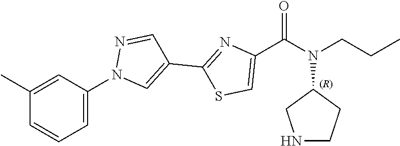 | 2-[1-(3-methylphenyl)-1H- pyrazol-4-yl]-N-propyl-N- [(3R)-pyrrolidin-3-yl]-1,3- thiazole-4-carboxamide |
| 31 | 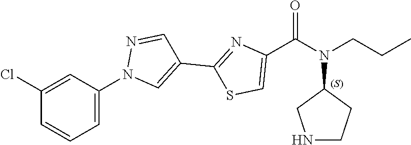 | 2-[1-(3-chlorophenyl)-1H- pyrazol-4-yl]-N-propyl-N- [(3S)-pyrrolidin-3-yl]-1,3- thiazole-4-carboxamide |
| 32 | 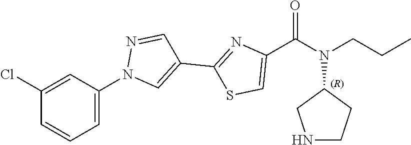 | 2-[1-(3-chlorophenyl)-1H- pyrazol-4-yl]-N-propyl-N- [(3R)-pyrrolidin-3-yl]-1,3- thiazole-4-carboxamide |
| 35 | 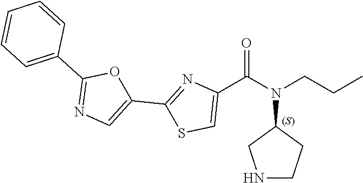 | 2-(2-phenyl-1,3-oxazol-5- yl)-N-propyl-N-[(3S)- pyrrolidin-3-yl]-1,3- thiazole-4-carboxamide |
| 36 | 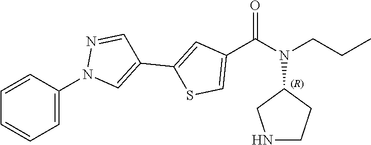 | 5-(1-phenyl-1H-pyrazol-4- yl)-N-propyl-N-[(3R)- pyrrolidin-3-yl]thiophene-3- carboxamide |
| 40 | 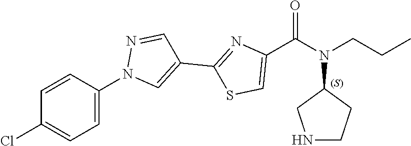 | 2-[1-(4-chlorophenyl)-1H- pyrazol-4-yl]-N-propyl-N- [(3S)-pyrrolidin-3-yl]-1,3- thiazole-4-carboxamide |
| 41 | 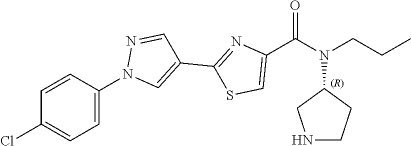 | 2-[1-(4-chlorophenyl)-1H- pyrazol-4-yl]-N-propyl-N- [(3R)-pyrrolidin-3-yl]-1,3- thiazole-4-carboxamide |
| 42 | 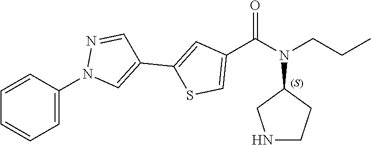 | 5-(1-phenyl-1H-pyrazol-4- yl)-N-propyl-N-[(3S)- pyrrolidin-3-yl]thiophene-3- carboxamide |
| 43 | 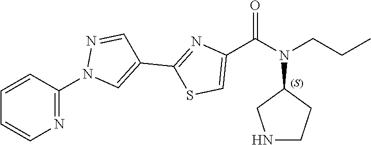 | N-propyl-2-[1-(pyridin-2- yl)-1H-pyrazol-4-yl]-N- [(3S)-pyrrolidin-3-yl]-1,3- thiazole-4-carboxamide |
| 44 | 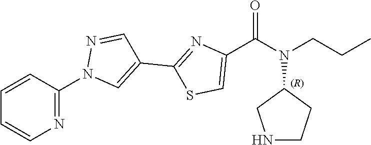 | N-propyl-2-[1-(pyridin-2- yl)-1H-pyrazol-4-yl]-N- [(3R)-pyrrolidin-3-yl]-1,3- thiazole-4-carboxamide |
| 45 | 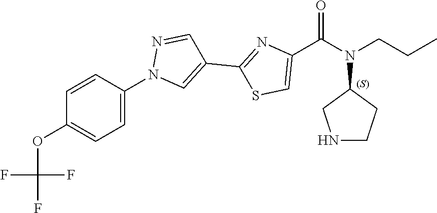 | N-propyl-N-[(3S)- pyrrolidin-3-yl]-2-{1-[4- (trifluoromethoxy)phenyl]- 1H-pyrazol-4-yl}-1,3- thiazole-4-carboxamide |
| 46 | 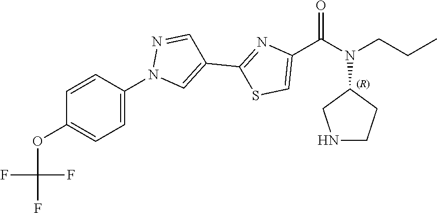 | N-propyl-N-[(3R)- pyrrolidin-3-yl]-2-{1-[4- (trifluoromethoxy)phenyl]- 1H-pyrazol-4-yl}-1,3- thiazole-4-carboxamide |
| 47 | 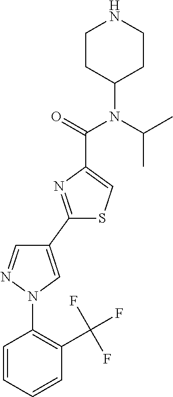 | N-(piperidin-4-yl)-N- (propan-2-yl)-2-{1-[2- (trifluoromethyl)phenyl]- 1H-pyrazol-4-yl}-1,3- thiazole-4-carboxamide |
| 48 | 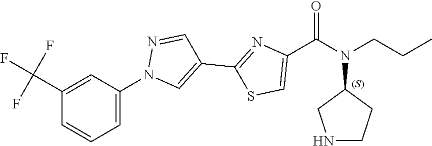 | N-propyl-N-[(3S)- pyrrolidin-3-yl]-2-{1-[3- (trifluoromethyl)phenyl]- 1H-pyrazol-4-yl}-1,3- thiazole-4-carboxamide |
| 49 | 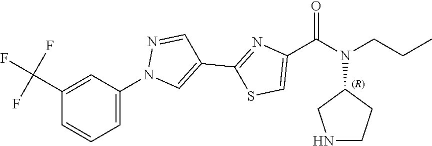 | N-propyl-N-[(3R)- pyrrolidin-3-yl]-2-{1-[3- (trifluoromethyl)phenyl]- 1H-pyrazol-4-yl}-1,3- thiazole-4-carboxamide |
| 50 | 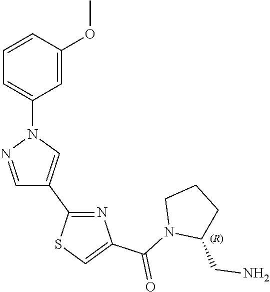 | 1-[(2R)-1-{2-[1-(3- methoxyphenyl)-1H- pyrazol-4-yl]-1,3-thiazole-4- carbonyl}pyrrolidin-2- yl]methanamine |
| 51 | 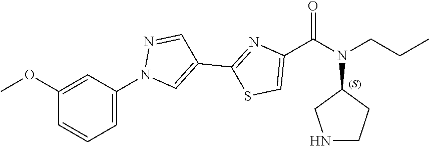 | 2-[1-(3-methoxyphenyl)- 1H-pyrazol-4-yl]-N-propyl- N-[(3S)-pyrrolidin-3-yl]- 1,3-thiazole-4-carboxamide |
| 52 | 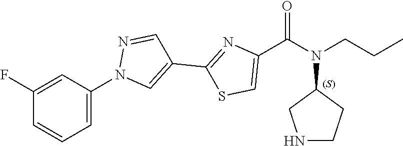 | 2-[1-(3-fluorophenyl)-1H- pyrazol-4-yl]-N-propyl-N- [(3S)-pyrrolidin-3-yl]-1,3- thiazole-4-carboxamide |
| 53 | 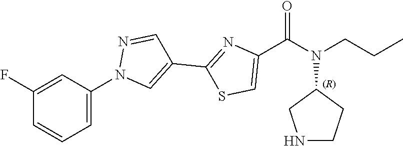 | 2-[1-(3-fluorophenyl)-1H- pyrazol-4-yl]-N-propyl-N- [(3R)-pyrrolidin-3-yl]-1,3- thiazole-4-carboxamide |
| 54 | 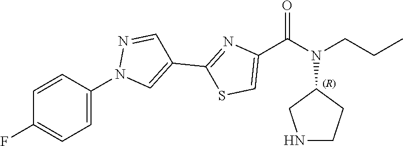 | 2-[1-(4-fluorophenyl)-1H- pyrazol-4-yl]-N-propyl-N- [(3R)-pyrrolidin-3-yl]-1,3- thiazole-4-carboxamide |
| 56 | 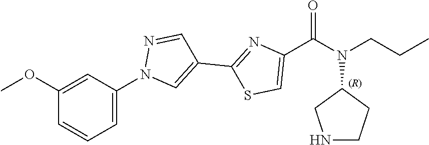 | 2-[1-(3-methoxyphenyl)- 1H-pyrazol-4-yl]-N-propyl- N-[(3R)-pyrrolidin-3-yl]- 1,3-thiazole-4-carboxamide |
| 57 | 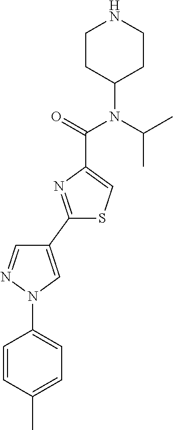 | 2-[1-(4-methylphenyl)-1H- pyrazol-4-yl]-N-(piperidin- 4-yl)-N-(propan-2-yl)-1,3- thiazole-4-carboxamide |
| 58 | 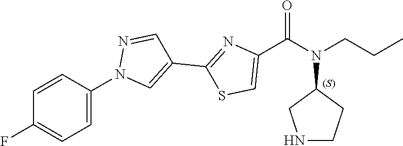 | 2-[1-(4-fluorophenyl)-1H- pyrazol-4-yl]-N-propyl-N- [(3S)-pyrrolidin-3-yl]-1,3- thiazole-4-carboxamide |
| 59 | 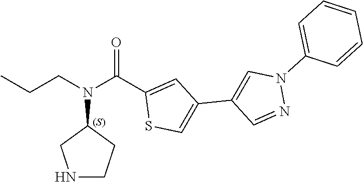 | 4-(1-phenyl-1H-pyrazol-4- yl)-N-propyl-N-[(3S)- pyrrolidin-3-yl]thiophene-2- carboxamide |
| 61 | 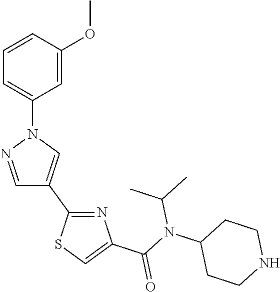 | 2-[1-(3-methoxyphenyl)- 1H-pyrazol-4-yl]-N- (piperidin-4-yl)-N-(propan- 2-yl)-1,3-thiazole-4-carboxamide |
| 64 | 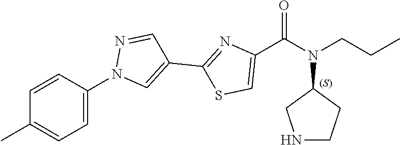 | 2-[1-(4-methylphenyl)-1H- pyrazol-4-yl]-N-propyl-N- [(3S)-pyrrolidin-3-yl]-1,3- thiazole-4-carboxamide |
| 65 | 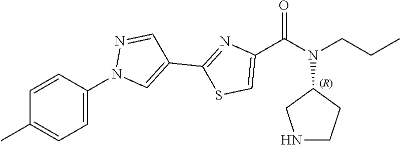 | 2-[1-(4-methylphenyl)-1H- pyrazol-4-yl]-N-propyl-N- [(3R)-pyrrolidin-3-yl]-1,3- thiazole-4-carboxamide |
| 66 | 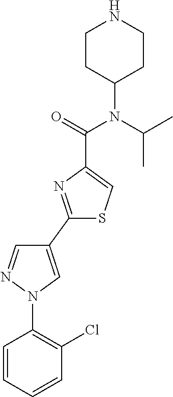 | 2-[1-(2-chlorophenyl)-1H- pyrazol-4-yl]-N-(piperidin- 4-yl)-N-(propan-2-yl)-1,3- thiazole-4-carboxamide |
| 68 | 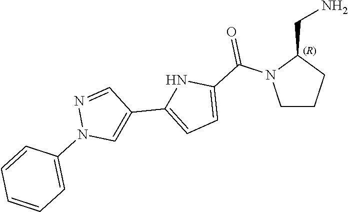 | 1-[(2R)-1-[5-(1-phenyl-1H- pyrazol-4-yl)-1H-pyrrole-2- carbonyl]pyrrolidin-2- yl]methanamine |
| 69 | 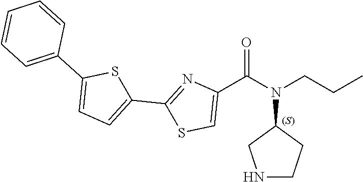 | 2-(5-phenylthiophen-2-yl)- N-propyl-N-[(3S)- pyrrolidin-3-yl]-1,3- thiazole-4-carboxamide |
| 70 | 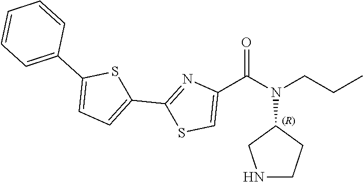 | 2-(5-phenylthiophen-2-yl)- N-propyl-N-[(3R)- pyrrolidin-3-yl]-1,3- thiazole-4-carboxamide |
| 71 | 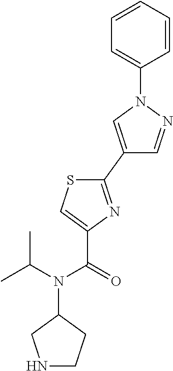 [SMILES: CC(C)N(C(=O)c1csc(-c2cnn(-c3ccccc3)c2)n1)C1CCNC1] | 2-(1-phenyl-1H-pyrazol- 4-yl)-N-(propan-2-yl)-N- (pyrrolidin-3-yl)-1,3- thiazole-4-carboxamide |
| 73 | 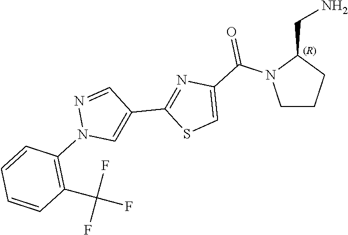 | 1-[(2R)-1-(2-{1-[2- (trifluoromethyl)phenyl]- 1H-pyrazol-4-yl}-1,3- thiazole-4- carbonyl)pyrrolidin-2- yl]methanamine |
| 74 | 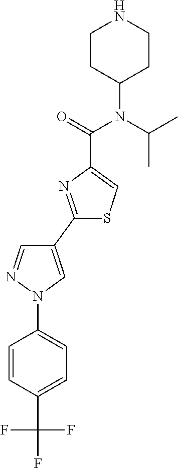 | N-(piperidin-4-yl)-N- (propan-2-yl)-2-{1-[4- (trifluoromethyl)phenyl]- 1H-pyrazol-4-yl}-1,3- thiazole-4-carboxamide |
| 76 | 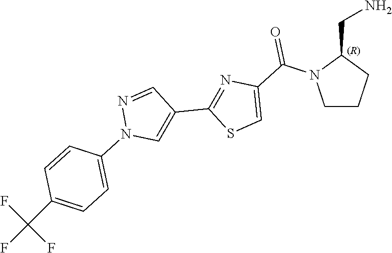 | 1-[(2R)-1-(2-{1-[4- (trifluoromethyl)phenyl]- 1H-pyrazol-4-yl}-1,3- thiazole-4- carbonyl)pyrrolidin-2- yl]methanamine |
| 77 | 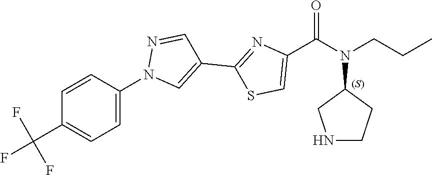 | N-propyl-N-[(3S)- pyrrolidin-3-yl]-2-{1-[4- (trifluoromethyl)phenyl]- 1H-pyrazol-4-yl}-1,3- thiazole-4-carboxamide |
| 78 | 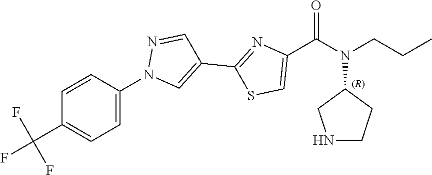 | N-propyl-N-[(3R)- pyrrolidin-3-yl]-2-{1-[4- (trifluoromethyl)phenyl]- 1H-pyrazol-4-yl}-1,3- thiazole-4-carboxamide |
| 80 | 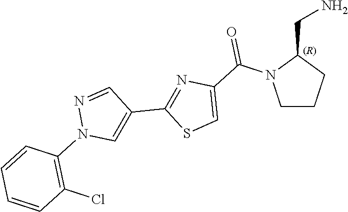 | 1-[(2R)-1-{2-[1-(2- chlorophenyl)-1H-pyrazol- 4-yl]-1,3-thiazole-4- carbonyl}pyrrolidin-2- yl]methanamine |
| 81 | 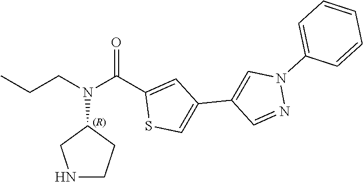 | 4-(1-phenyl-1H-pyrazol-4- yl)-N-propyl-N-[(3R)- pyrrolidin-3-yl]thiophene-2- carboxamide |
| 82 | 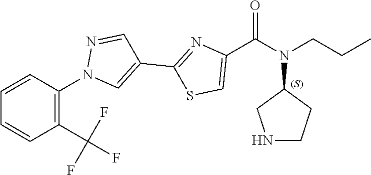 | N-propyl-N-[(3S)- pyrrolidin-3-yl]-2-{1-[2- (trifluoromethyl)phenyl]- 1H-pyrazol-4-yl}-1,3- thiazole-4-carboxamide |
| 83 | 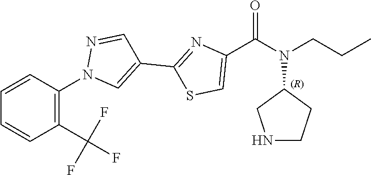 | N-propyl-N-[(3R)- pyrrolidin-3-yl]-2-{1-[2- (trifluoromethyl)phenyl]-1H-pyrazol- 4-yl}-1,3-thiazole-4-carboxamide |
| 84 | 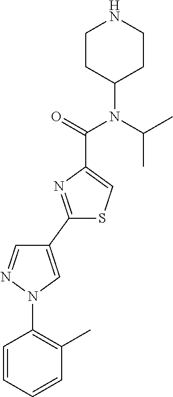 | 2-[1-(2-methylphenyl)-1H-pyrazol-4- yl]-N-(piperidin-4-yl)-N-(propan-2-yl)- 1,3-thiazole-4-carboxamide |
| 86 | 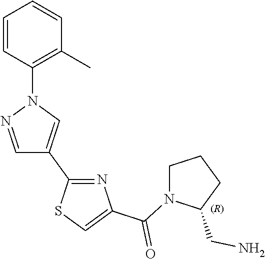 | 1-[(2R)-1-{2-[1-(2- methylphenyl)-1H-pyrazol-4-yl]-1,3- thiazole-4-carbonyl}pyrrolidin-2- yl]methanamine |
| 87 | 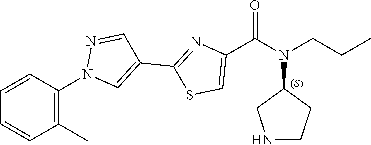 | 2-[1-(2-methylphenyl)-1H-pyrazol-4- yl]-N-propyl-N-[(3S)-pyrrolidin-3-yl]- 1,3-thiazole-4-carboxamide |
| 88 | 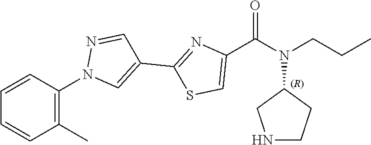 | 2-[1-(2-methylphenyl)-1H-pyrazol-4- yl]-N-propyl-N-[(3R)-pyrrolidin-3-yl]- 1,3-thiazole-4-carboxamide |
| 89 | 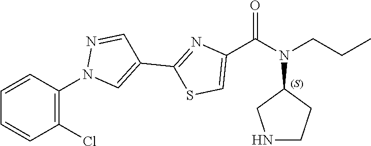 | 2-[1-(2-chlorophenyl)-1H-pyrazol-4- yl]-N-propyl-N-[(3S)-pyrrolidin-3-yl]- 1,3-thiazole-4-carboxamide |
| 90 | 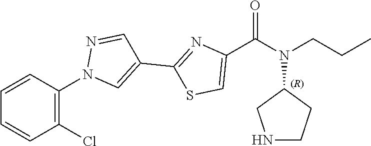 | 2-[1-(2-chlorophenyl)-1H-pyrazol-4- yl]-N-propyl-N-[(3R)-pyrrolidin-3-yl]- 1,3-thiazole-4-carboxamide |
| 91 | 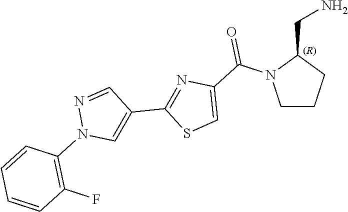 | 1-[(2R)-1-{2-[1-(2- fluorophenyl)-1H-pyrazol-4yl]-1,3- thiazole-4-carbonyl}pyrrolidin-2- yl]methanamine |
| 92 | 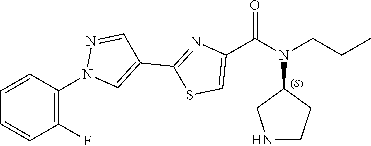 | 2-[1-(2-fluorophenyl)-1H-pyrazol-4- yl]-N-propyl-N-[(3S)-pyrrolidin-3-yl]- 1,3-thiazole-4-carboxamide |
| 93 | 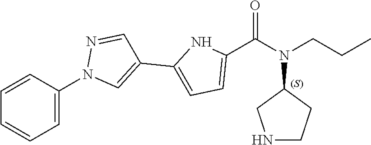 | 5-(1-phenyl-1H-pyrazol-4-yl)-N-propyl- N-[(3S)-pyrrolidin-3-yl]-1H-pyrrole-2- carboxamide |
| 94 | 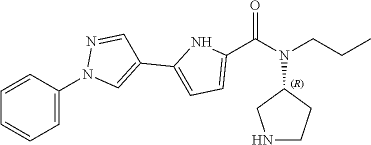 | 5-(1-phenyl-1H-pyrazol-4-yl)-N-propyl- N-[(3R)-pyrrolidin-3-yl]-1H-pyrrole-2- carboxamide |
| 95 | 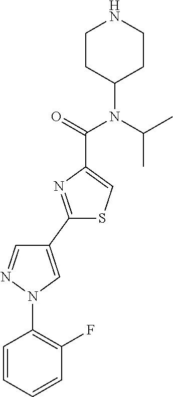 | 2-[1-(2-fluorophenyl)-1H-pyrazol-4- yl]-N-(piperidin-4-yl)-N-(propan-2-yl)- 1,3-thiazole-4-carboxamide |
| 97 | 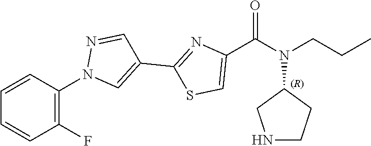 | 2-[1-(2-fluorophenyl)-1H-pyrazol-4- yl]-N-propyl-N-[(3R)-pyrrolidin-3-yl]- 1,3-thiazole-4-carboxamide |
| 98 | 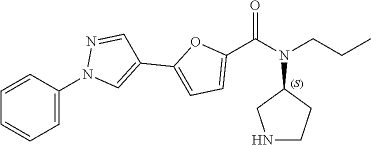 | 5-(1-phenyl-1H-pyrazol-4-yl)-N- propyl-N-[(3S)-pyrrolidin-3-yl]furan- 2-carboxamide |
| 99 | 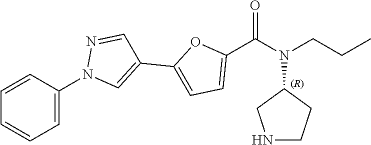 | 5-(1-phenyl-1H-pyrazol-4-yl)-N- propyl-N-[(3R)-pyrrolidin-3-yl]furan- 2-carboxamide |
| 100 | 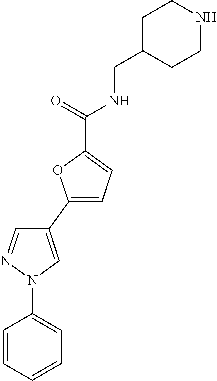 | 5-(1-phenyl-1H-pyrazol-4-yl)-N- [(piperidin-4-yl)methyl]furan-2- carboxamide |
| 101 | 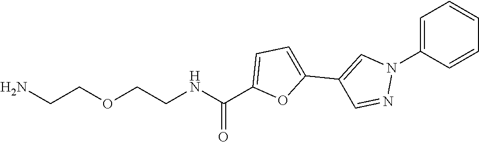 [SMILES: NCCOCCNC(=O)c1ccc(-c2cnn(-c3ccccc3)c2)o1] | N-[2-(2- aminoethoxy)ethyl]-5-(1-phenyl-1H- pyrazol-4-yl)furan-2-carboxamide |
| 104 | 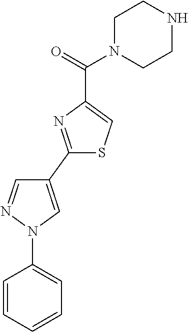 | 1-[2-(1-phenyl-1H-pyrazol- 4-yl)-1,3-thiazole-4- carbonyl]piperazine |
| 106 | 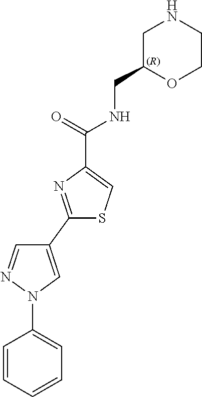 | N-{[(2R)-morpholin-2- yl]methyl}-2-(1-phenyl-1H-pyrazol-4- yl)-1,3-thiazole-4-carboxamide |
| 107 | 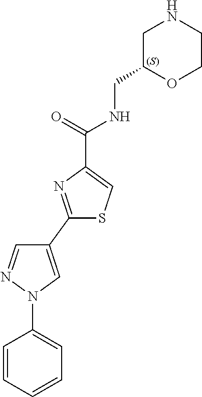 | N-{[(2S)-morpholin-2- yl]methyl}-2-(1-phenyl-1H-pyrazol-4- yl)-1,3-thiazole-4carboxamide |
| 108 | 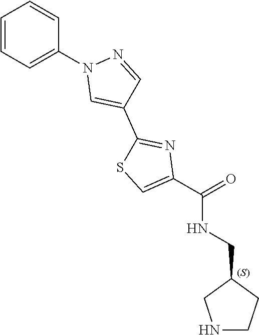 | 2-(1-phenyl-1H-pyrazol-4-yl)-N-{[(3S)- pyrrolidin-3-yl]methyl}-1,3-thiazole-4- carboxamide |
| 110 | 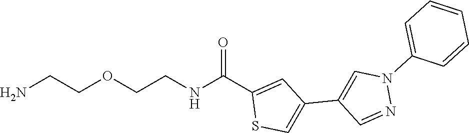 [SMILES: NCCOCCNC(=O)c1cc(-c2cnn(-c3ccccc3)c2)cs1] | N-[2-(2-aminoethoxy)ethyl]-4-(1-phenyl- 1H-pyrazol-4-yl)thiophene-2- carboxamide |
| 111 | 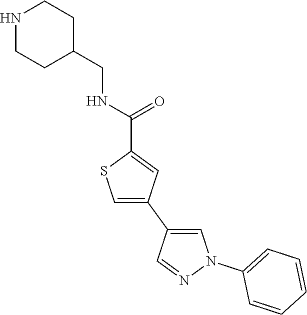 [SMILES: O=C(NCC1CCNCC1)c1cc(-c2cnn(-c3ccccc3)c2)cs1] | 4-(1-phenyl-1H-pyrazol-4-yl)-N- [(piperidin-4-yl)methyl]thiophene-2- carboxamide |
| 112 | 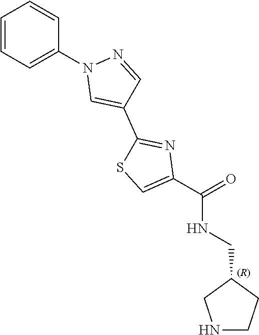 | 2-(1-phenyl-1H-pyrazol-4-yl)-N- {[(3R)-pyrrolidin-3-yl]methyl}-1,3- thiazole-4-carboxamide |
| 114 | 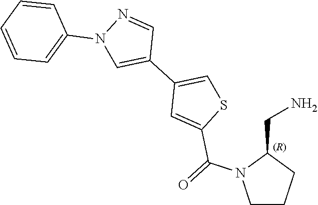 | 1-[(2R)-1-[4-(1-phenyl-1H- pyrazol-4-yl)thiophene-2- carbonyl]pyrrolidin-2- yl]methanamine |
| 115 | 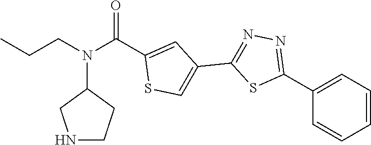 [SMILES: CCCN(C(=O)c1cc(-c2nnc(-c3ccccc3)s2)cs1)C1CCNC1] | 4-(5-phenyl-1,3,4-thiadiazol-2-yl)-N- propyl-N-(pyrrolidin-3-yl)thiophene- 2-carboxamide |
| 116 | 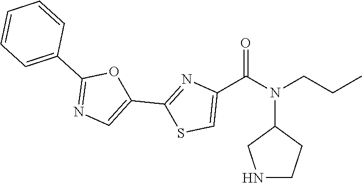 [SMILES: CCCN(C(=O)c1csc(-c2cnc(-c3ccccc3)o2)n1)C1CCNC1] | 2-(2-phenyl-1,3-oxazol-5-yl)-N-propyl- N-(pyrrolidin-3-yl)-1,3-thiazole-4- carboxamide |
| 117 | 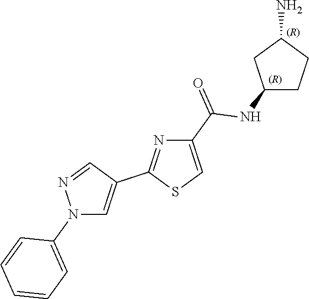 | N-[(1R,3R)-3- aminocyclopentyl]-2-(1-phenyl-1H- pyrazol-4-yl)-1,3-thiazole-4- carboxamide |
| 119 | 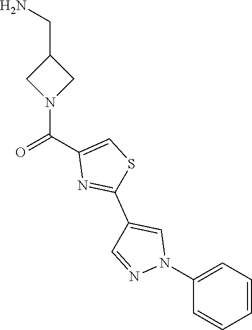 [SMILES: NCC1CN(C(=O)c2csc(-c3cnn(-c4ccccc4)c3)n2)C1] | 1-{1-[2-(1-phenyl-1H- pyrazol-4-yl)-1,3-thiazole-4- carbonyl]azetidin-3-yl}methanamine |
| 120 | 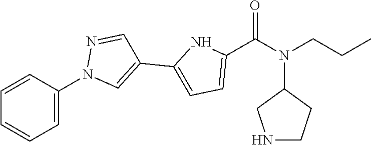 [SMILES: CCCN(C(=O)c1ccc(-c2cnn(-c3ccccc3)c2)[nH]1)C1CCNC1] | 5-(1-phenyl-1H-pyrazol-4-yl)-N-propyl- N-(pyrrolidin-3-yl)-1H-pyrrole-2- carboxamide |
| 121 | 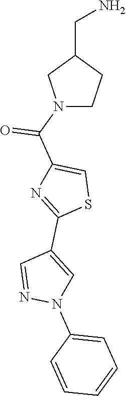 [SMILES: NCC1CCN(C(=O)c2csc(-c3cnn(-c4ccccc4)c3)n2)C1] | 1-{1-[2-(1-phenyl-1H- pyrazol-4-yl)-1,3-thiazole-4- carbonyl]pyrrolidin-3-yl}methanamine |
| 122 | 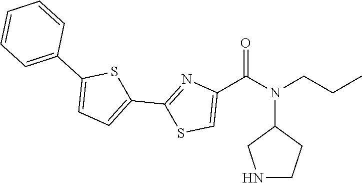 [SMILES: CCCN(C(=O)c1csc(-c2ccc(-c3ccccc3)s2)n1)C1CCNC1] | 2-(5-phenylthiophen-2-yl)-N-propyl-N- (pyrrolidin-3-yl)-1,3-thiazole-4- carboxamide |
| 123 | 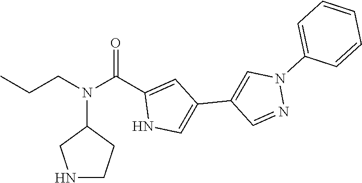 [SMILES: CCCN(C(=O)c1cc(-c2cnn(-c3ccccc3)c2)c[nH]1)C1CCNC1] | 4-(1-phenyl-1H-pyrazol-4-yl)-N-propyl- N-(pyrrolidin-3-yl)-1H-pyrrole-2- carboxamide |
| 124 | 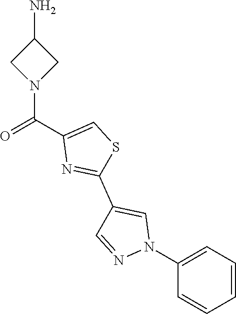 [SMILES: NC1CN(C(=O)c2csc(-c3cnn(-c4ccccc4)c3)n2)C1] | 1-[2-(1-phenyl-1H-pyrazol-4-yl)-1,3- thiazole-4-carbonyl]azetidin-3-amine |
| 126 | 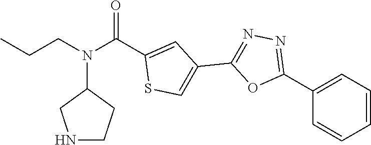 [SMILES: CCCN(C(=O)c1cc(-c2nnc(-c3ccccc3)o2)cs1)C1CCNC1] | 4-(5-phenyl-1,3,4-oxadiazol-2-yl)-N- propyl-N(pyrrolidin-3-yl)thiophene-2- carboxamide |
| 127 | 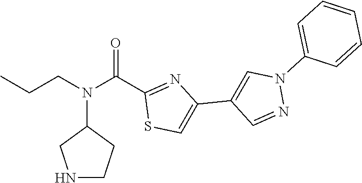 [SMILES: CCCN(C(=O)c1nc(-c2cnn(-c3ccccc3)c2)cs1)C1CCNC1] | 4-(1-phenyl-1H-pyrazol-4-yl)-N-propyl- N-(pyrrolidin-3-yl)-1,3-thiazole-2- carboxamide |
| 128 | 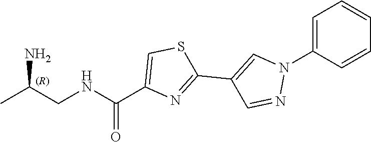 | N-[(2R)-2-aminopropyl]-2-(1-phenyl- 1H-pyrazol-4-yl)-1,3-thiazole-4- carboxamide |
| 129 | 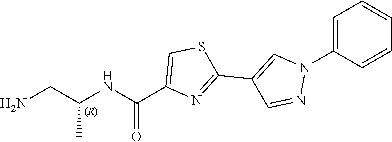 | N-[(2R)-1-aminopropan-2-yl]-2-(1- phenyl-1H-pyrazol-4-yl)-1,3-thiazole-4- carboxamide |
| 130 | 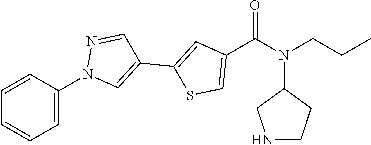 [SMILES: CCCN(C(=O)c1csc(-c2cnn(-c3ccccc3)c2)c1)C1CCNC1] | 5-(1-phenyl-1H-pyrazol-4-yl)-N-propyl- N-(pyrrolidin-3-yl)thiophene-3- carboxamide |
| 131 | 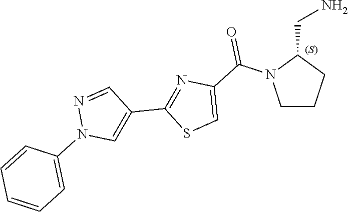 | 1-[(2S)-1-[2-(1-phenyl-1H- pyrazol-4-yl)-1,3-thiazole-4- carbonyl]pyrrolidin-2-yl]methanamine |
| 132 | 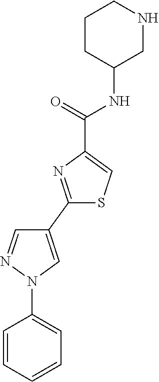 [SMILES: O=C(NC1CCCNC1)c1csc(-c2cnn(-c3ccccc3)c2)n1] | 2-(1-phenyl-1H-pyrazol-4-yl)-N- (piperidin-3-yl)-1,3-thiazole-4- carboxamide |
| 133 | 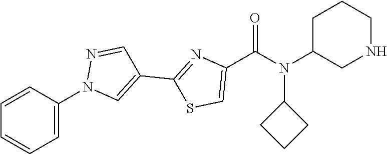 [SMILES: O=C(c1csc(-c2cnn(-c3ccccc3)c2)n1)N(C1CCC1)C1CCCNC1] | N-cyclobutyl-2-(1-phenyl-1H-pyrazol- 4-yl)-N- (piperidin-3-yl)-1,3-thiazole-4- carboxamide |
| 135 | 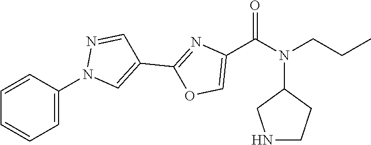 [SMILES: CCCN(C(=O)c1coc(-c2cnn(-c3ccccc3)c2)n1)C1CCNC1] | 2-(1-phenyl-1H-pyrazol-4-yl)-N-propyl- N-(pyrrolidin-3-yl)-1,3-oxazole-4- carboxamide |
| 136 | 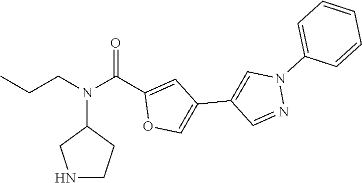 [SMILES: CCCN(C(=O)c1cc(-c2cnn(-c3ccccc3)c2)co1)C1CCNC1] | 4-(1-phenyl-1H-pyrazol-4-yl)-N-propyl- N-(pyrrolidin-3-yl)furan-2-carboxamide |
| 137 | 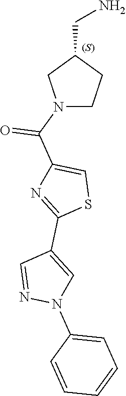 | 1-[(3S)-1-[2-(1-phenyl-1H- pyrazol-4-yl)-1,3-thiazole-4- carbonyl]pyrrolidine-3-yl]methanamine |
| 139 | 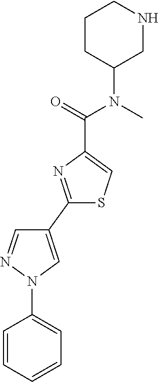 [SMILES: CN(C(=O)c1csc(-c2cnn(-c3ccccc3)c2)n1)C1CCCNC1] | N-methyl-2-(1-phenyl-1H-pyrazol-4- yl)-N-(piperidin-3-yl)-1,3-thiazole-4- carboxamide |
| 140 | 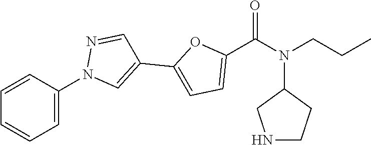 [SMILES: CCCN(C(=O)c1ccc(-c2cnn(-c3ccccc3)c2)o1)C1CCNC1] | 5-(1-phenyl-1H-pyrazol-4-yl)-N-propyl- N-(pyrrolidin-3-yl)furan-2-carboxamide |
| 141 | 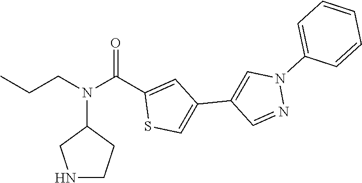 [SMILES: CCCN(C(=O)c1cc(-c2cnn(-c3ccccc3)c2)cs1)C1CCNC1] | 4-(1-phenyl-1H-pyrazol-4-yl)-N-propyl- N-(pyrrolidin-3-yl)thiophene-2- carboxamide |
| 142 | 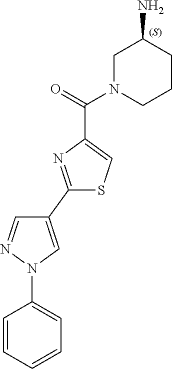 | (3S)-1-[2-(1-phenyl-1H- pyrazol-4-yl)-1,3-thiazole-4- carbonyl]piperidin-3-amine |
| 143 | 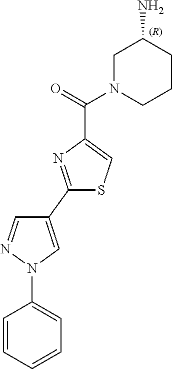 | (3R)-1-[2-(1-phenyl-1H- pyrazol-4-yl)-1,3-thiazole-4- carbonyl]piperidin-3-amine |
| 145 | 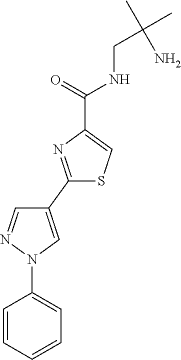 [SMILES: CC(C)(N)CNC(=O)c1csc(-c2cnn(-c3ccccc3)c2)n1] | N-(2-amino-2-methylpropyl)-2-(1- phenyl-1H-pyrazol-4-yl)-1,3-thiazole-4- carboxamide |
| 146 | 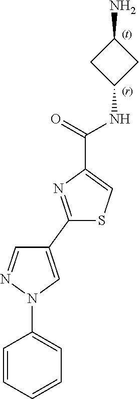 | 2-(1-phenyl-1H-pyrazol-4-yl)-N- [(1r,3r)-3- aminocyclobutyl]-1,3-thiazole- 4-carboxamide |
| 149 | 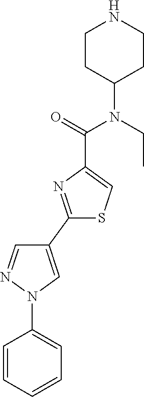 [SMILES: CCN(C(=O)c1csc(-c2cnn(-c3ccccc3)c2)n1)C1CCNCC1] | N-ethyl-2-(1-phenyl-1H-pyrazol-4-yl)- N-(piperidin-4-yl)-1,3-thiazole-4- carboxamide |
| 150 | 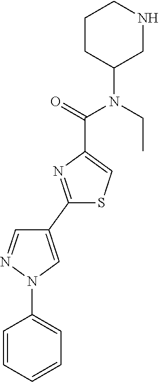 [SMILES: CCN(C(=O)c1csc(-c2cnn(-c3ccccc3)c2)n1)C1CCCNC1] | N-ethyl-2-(1-phenyl-1H-pyrazol-4-yl)- N-(piperidin-3-yl)-1,3-thiazole-4- carboxamide |
| 152 | 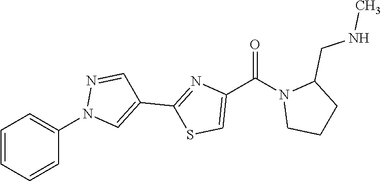 [SMILES: CNCC1CCCN1C(=O)c1csc(-c2cnn(-c3ccccc3)c2)n1] | methyl({1-[2-(1-phenyl-1H-pyrazol-4- yl)-1,3-thiazole-4carbonyl]pyrrolidin-2- yl}methyl)amine |
| 153 | 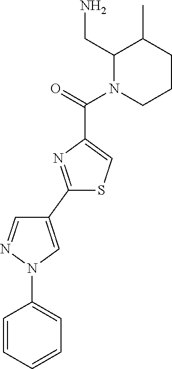 [SMILES: CC1CCCN(C(=O)c2csc(-c3cnn(-c4ccccc4)c3)n2)C1CN] | 1-{3-methyl-1-[2-(1-phenyl-1H-pyrazol- 4-yl)-1,3-thiazole-4-carbonyl]piperidin- 2-yl}methanamine |
| 154 | 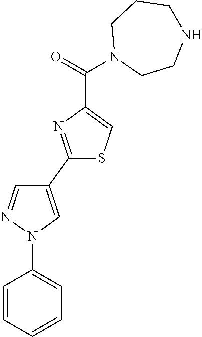 [SMILES: O=C(c1csc(-c2cnn(-c3ccccc3)c2)n1)N1CCCNCC1] | 1-[2-(1-phenyl-1H-pyrazol- 4-yl)-1,3-thiazole-4-carbonyl]-1,4- diazepane |
| 156 | 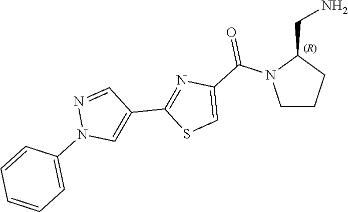 | 1-[(2R)-1-[2-(1-phenyl-1H- pyrazol-4-yl)-1,3-thiazole-4- carbonyl]pyrrolidine-2-yl]methanamine |
| 158 | 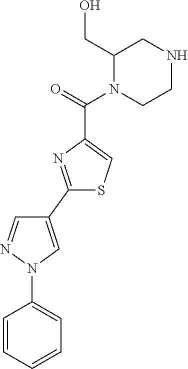 [SMILES: O=C(c1csc(-c2cnn(-c3ccccc3)c2)n1)N1CCNCC1CO] | {1-[2-(1-phenyl-1H-pyrazol 4-yl)-1,3-thiazole-4- carbonyl]piperazin-2-yl}methanol |
| 159 | 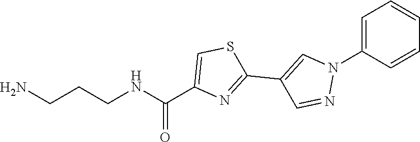 [SMILES: NCCCNC(=O)c1csc(-c2cnn(-c3ccccc3)c2)n1] | N-(3-aminopropyl)-2-(1-phenyl-1H- pyrazol-4-yl)-1,3-thiazole-4- carboxamide |
| 160 | 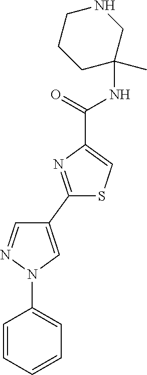 [SMILES: CC1(NC(=O)c2csc(-c3cnn(-c4ccccc4)c3)n2)CCCNC1] | N-(3-methylpiperidin-3-yl)-2-(1-phenyl- 1H-pyrazol-4-yl)-1,3-thiazole-4- carboxamide |
| 161 | 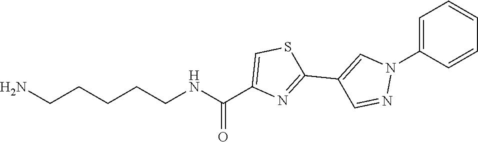 [SMILES: NCCCCCNC(=O)c1csc(-c2cnn(-c3ccccc3)c2)n1] | N-(5-aminopentyl)-2-(1-phenyl-1H- pyrazol-4-yl)-1,3-thiazole-4- carboxamide |
| 162 | 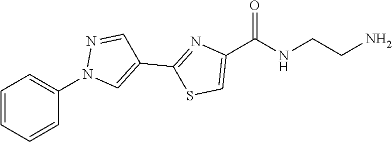 [SMILES: NCCNC(=O)c1csc(-c2cnn(-c3ccccc3)c2)n1] | N-(2-aminoethyl)-2-(1-phenyl-1H- pyrazol-4-yl)-1,3-thiazole-4- carboxamide |
| 163 | 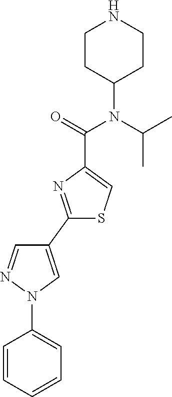 [SMILES: CC(C)N(C(=O)c1csc(-c2cnn(-c3ccccc3)c2)n1)C1CCNCC1] | 2-(1-phenyl-1H-pyrazol-4-yl)-N- (piperidin-4-yl)-N-(propan-2-yl)-1,3- thiazole-4-carboxamide |
| 165 | 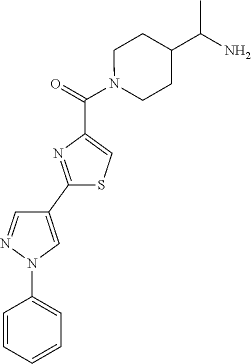 [SMILES: CC(N)C1CCN(C(=O)c2csc(-c3cnn(-c4ccccc4)c3)n2)CC1] | 1-{1-[2-(1-phenyl-1H- pyrazol-4-yl)-1,3-thiazole-4 carbonyl]piperidin-4-yl}ethan-1-amine |
| 167 | 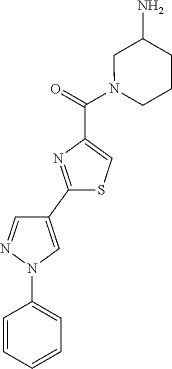 [SMILES: NC1CCCN(C(=O)c2csc(-c3cnn(-c4ccccc4)c3)n2)C1] | 1-[2-(1-phenyl-1H-pyrazol- 4-yl)-1,3-thiazole-4-carbonyl]piperidin- 3-amine |
| 170 | 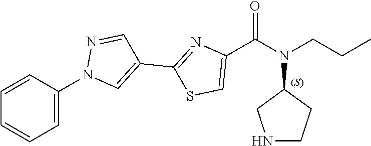 | 2-(1-phenyl-1H-pyrazol-4-yl)-N- propyl-N-[(3S)-pyrrolidin-3-yl]-1,3- thiazole-4-carboxamide |
| 171 | 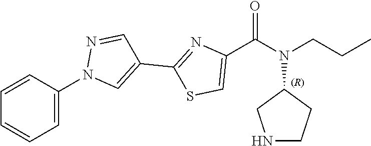 | 2-(1-phenyl-1H-pyrazol-4-yl)-N- propyl-N-[(3R)-pyrrolidin-3-yl]-1,3- thiazole-4-carboxamide |
| 175 | 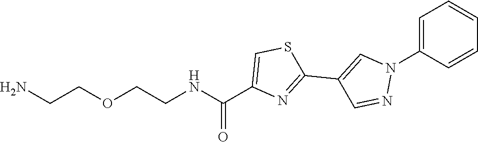 [SMILES: NCCOCCNC(=O)c1csc(-c2cnn(-c3ccccc3)c2)n1] | N-[2-(2- aminoethoxy)ethyl]-2-(1-phenyl-1H- pyrazol-4-yl)-1,3-thiazole-4- carboxamide |
| 177 | 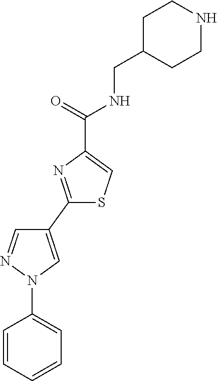 [SMILES: O=C(NCC1CCNCC1)c1csc(-c2cnn(-c3ccccc3)c2)n1] | 2-(1-phenyl-1H-pyrazol-4-yl)-N- [(piperidin-4-yl)methyl]-1,3-thiazole- 4-carboxamide |
| 178 | 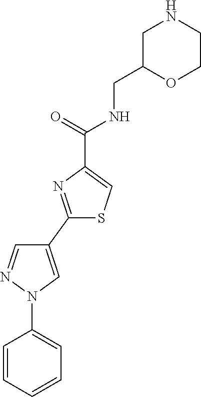 [SMILES: O=C(NCC1CNCCO1)c1csc(-c2cnn(-c3ccccc3)c2)n1] | N-[(morpholin-2-yl)methyl]-2-(1- phenyl-1H-pyrazol-4-yl)-1,3-thiazole-4- carboxamide |
| 179 | 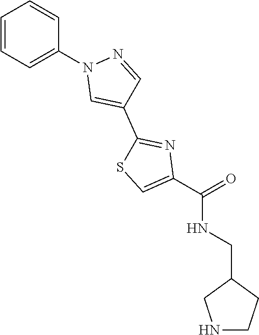 [SMILES: O=C(NCC1CCNC1)c1csc(-c2cnn(-c3ccccc3)c2)n1] | 2-(1-phenyl-1H-pyrazol-4-yl)-N- [(pyrrolidin-3-yl)methyl]-1,3-thiazole- 4-carboxamide |
| 180 | 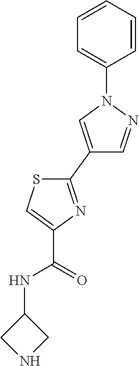 [SMILES: O=C(NC1CNC1)c1csc(-c2cnn(-c3ccccc3)c2)n1] | N-(azetidin-3-yl)-2-(1-phenyl-1H- pyrazol-4-yl)-1,3-thiazole-4- carboxamide |
| 181 | 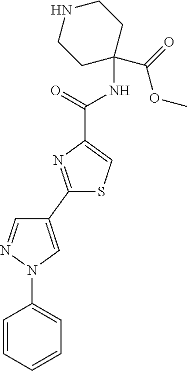 [SMILES: COC(=O)C1(NC(=O)c2csc(-c3cnn(-c4ccccc4)c3)n2)CCNCC1] | methyl 4-[2-(1-phenyl-1H-pyrazol-4- yl)-1,3-thiazole-4amido]piperidine-4- carboxylate |
| 183 | 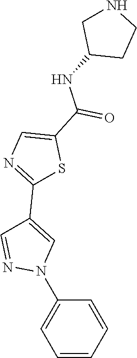 | 2-(1-phenyl-1H-pyrazol-4-yl)-N-[(3R)- pyrrolidin-3-yl]-1,3-thiazole-4- carboxamide |
| 184 | 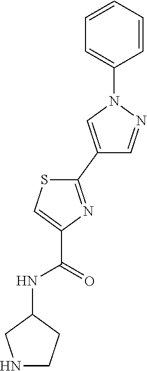 [SMILES: O=C(NC1CCNC1)c1csc(-c2cnn(-c3ccccc3)c2)n1] | 2-(1-phenyl-1H-pyrazol-4-yl)-N- (pyrrolidin-3-yl)-1,3-thiazole-4- carboxamide |
| Compound/ | | |
| Formula Number | | Chemical Name |
| 305 | 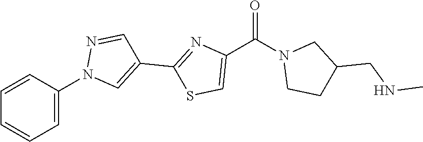 [SMILES: CNCC1CCN(C(=O)c2csc(-c3cnn(-c4ccccc4)c3)n2)C1] | methyl({1-[2-(1-phenyl-1H- pyrazol-4-yl)-1,3-thiazole-4- carbonyl]pyrrolidin-3- yl}methyl)amine |
| 306 | 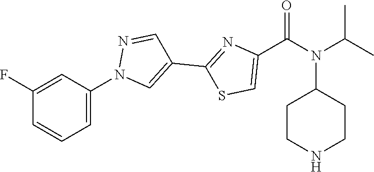 [SMILES: CC(C)N(C(=O)c1csc(-c2cnn(-c3cccc(F)c3)c2)n1)C1CCNCC1] | 2-[1-(3-fluorophenyl)-1H- pyrazol-4-yl]-N-(piperidin-4- yl)-N-(propan-2-yl)-1,3- thiazole-4-carboxamide |
| 307 | 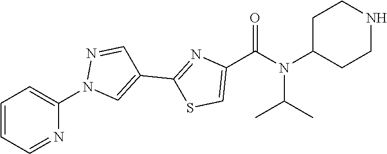 [SMILES: CC(C)N(C(=O)c1csc(-c2cnn(-c3ccccn3)c2)n1)C1CCNCC1] | N-(piperidin-4-yl)-N-(propan- 2-yl)-2-[1-(pyridin-2-yl)-1H- pyrazol-4-yl]-1,3-thiazole-4-carboxamide |
| 308 | 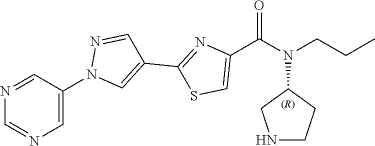 | N-propyl-2-[1-(pyrimidin-5- yl)-1H-pyrazol-4-yl]-N-[(3R)- pyrrolidin-3-yl]-1,3-thiazole-4-carboxamide |
| 309 | 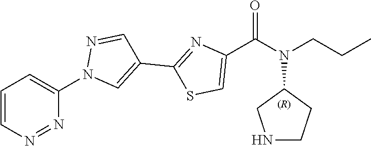 | N-propyl-2-[1-(pyridazin-3-yl)- 1H-pyrazol-4-yl]-N-[(3R)- pyrrolidin-3-yl]-1,3-thiazole-4- carboxamide |
| 310 | 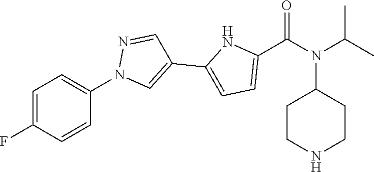 [SMILES: CC(C)N(C(=O)c1ccc(-c2cnn(-c3ccc(F)cc3)c2)[nH]1)C1CCNCC1] | 5-[1-(4-fluorophenyl)-1H- pyrazol-4-yl]-N-(piperidin-4- yl)-N-(propan-2-yl)-1H- pyrrole-2-carboxamide |
| 311 | 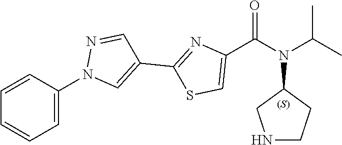 | 2-(1-phenyl-1H-pyrazol-4-yl)- N-(propan-2-yl)-N-[(3S)- pyrrolidin-3-yl]-1,3-thiazole-4- carboxamide |
| 312 | 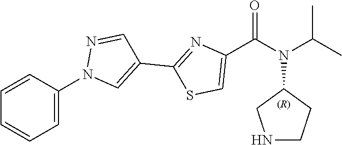 | 2-(1-phenyl-1H-pyrazol-4-yl)- N-(propan-2-yl)-N-[(3R)- pyrrolidin-3-yl]-1,3-thiazole-4- carboxamide |
| 313 | 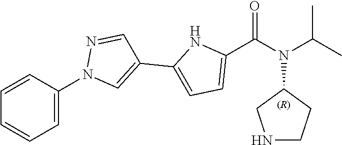 | 5-(1-phenyl-1H-pyrazol-4-yl)- N-(propan-2-yl)-N-[(3R)- pyrrolidin-3-yl]-1H-pyrrole-2- carboxamide |
| 314 | 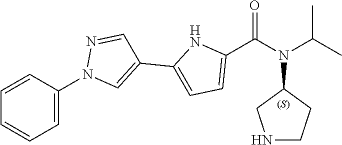 | 5-(1-phenyl-1H-pyrazol-4-yl)- N-(propan-2-yl)-N-[(3S)- pyrrolidin-3-yl]-1H-pyrrole-2- carboxamide |
| 315 | 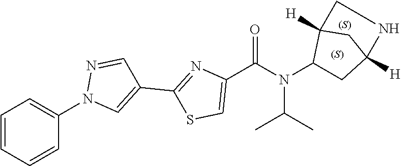 | N-[(1S,4S)-2- azabicyclo[2.2.1]heptan-5-yl]- 2-(1-phenyl-1H-pyrazol-4-yl)- N-(propan-2-yl)-1,3-thiazole- 4-carboxamide |
| 316 | 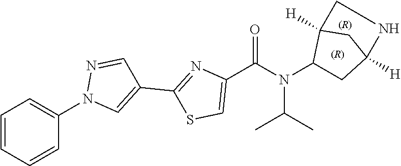 | N-[(1R,4R)-2- azabicyclo[2.2.1]heptan-5-yl]- 2-(1-phenyl-1H-pyrazol-4-yl)- N-(propan-2-yl)-1,3-thiazole- 4-carboxamide |
| 317 | 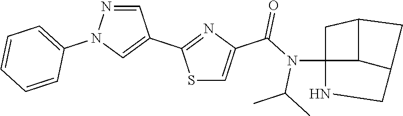 [SMILES: CC(C)N(C(=O)c1csc(-c2cnn(-c3ccccc3)c2)n1)C12CC3CC(CN1)C32] | N-{3-azabicyclo[3.1.1]heptan- 6-yl}-2-(1-phenyl-1H-pyrazol- 4-yl)-N-(propan-2-yl)-1,3- thiazole-4-carboxamide |
| 318 | 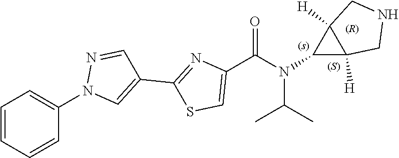 | N-[(1R,5S,6S)-3- azabicyclo[3.1.0]hexan-6-yl]- 2-(1-phenyl-1H-pyrazol-4-yl)- N-(propan-2-yl)-1,3-thiazole- 4-carboxamide |
| 319 | 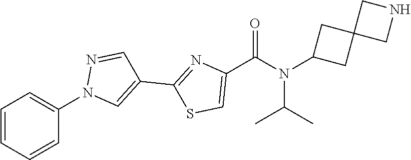 [SMILES: CC(C)N(C(=O)c1csc(-c2cnn(-c3ccccc3)c2)n1)C1CC2(CNC2)C1] | N-{2-azaspiro[3.3]heptan-6- yl}-2-(1-phenyl-1H-pyrazol-4- yl)-N-(propan-2-yl)-1,3- thiazole-4-carboxamide |
| 320 | 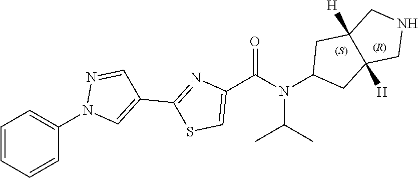 | N-[(3aR,6aS)- octahydrocyclopenta[c]pyrrol- 5-yl]-2-(1-phenyl-1H-pyrazol- 4-yl)-N-(propan-2-yl)-1,3- thiazole-4-carboxamide |
| 321 | 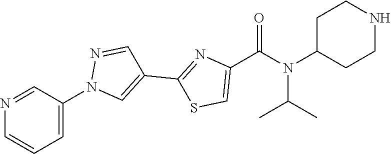 [SMILES: CC(C)N(C(=O)c1csc(-c2cnn(-c3cccnc3)c2)n1)C1CCNCC1] | N-(piperidin-4-yl)-N-(propan- 2-yl)-2-[1-(pyridin-3-yl)-1H- pyrazol-4-yl]-1,3-thiazole-4- carboxamide |
| 322 | 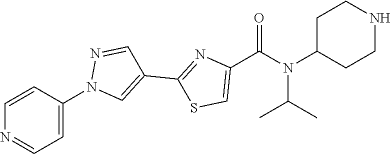 [SMILES: CC(C)N(C(=O)c1csc(-c2cnn(-c3ccncc3)c2)n1)C1CCNCC1] | N-(piperidin-4-yl)-N-(propan- 2-yl)-2-[1-(pyridin-4-yl)-1H- pyrazol-4-yl]-1,3-thiazole-4- carboxamide |
| 323 | 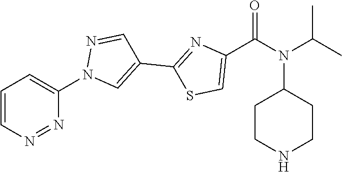 [SMILES: CC(C)N(C(=O)c1csc(-c2cnn(-c3cccnn3)c2)n1)C1CCNCC1] | N-(piperidin-4-yl)-N-(propan- 2-yl)-2-[1-(pyridazin-3-yl)-1H- pyrazol-4-yl]-1,3-thiazole-4- carboxamide |
| 324 | 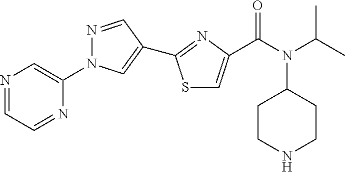 [SMILES: CC(C)N(C(=O)c1csc(-c2cnn(-c3cnccn3)c2)n1)C1CCNCC1] | N-(piperidin-4-yl)-N-(propan- 2-yl)-2-[1-(pyrazin-2-yl)-1H- pyrazol-4-yl]-1,3-thiazole-4- carboxamide |
| 325 | 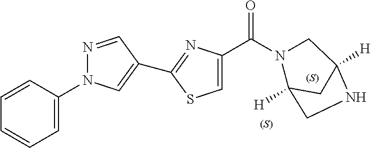 | (1S,4S)-2-[2-(1-phenyl-1H- pyrazol-4-yl)-1,3-thiazole-4- carbonyl]-2,5- diazabicyclo[2.2.1]heptane |
| 326 | 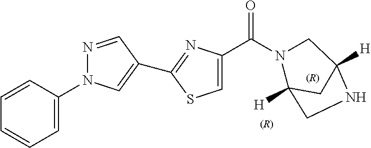 | (1R,4R)-2-[2-(1-phenyl-1H- pyrazol-4-yl)-1,3-thiazole-4- carbonyl]-2,5- diazabicyclo[2.2.1]heptane |
| 327 | 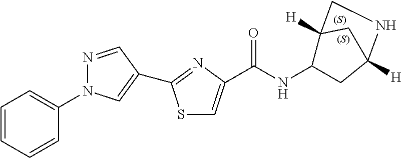 | N-[(1S,4S)-2- azabicyclo[2.2.1]heptan-5-yl]- 2-(1-phenyl-1H-pyrazol-4-yl)- 1,3-thiazole-4-carboxamide |
| 328 | 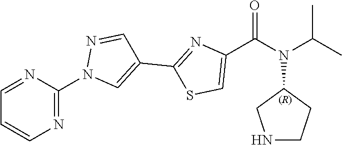 | N-(propan-2-yl)-2-[1- (pyrimidin-2-yl)-1H-pyrazol-4- yl]-N-[(3R)-pyrrolidin-3-yl]- 1,3-thiazole-4-carboxamide |
| 329 | 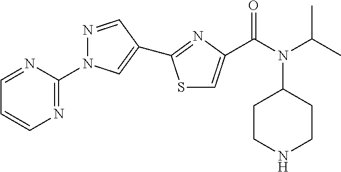 [SMILES: CC(C)N(C(=O)c1csc(-c2cnn(-c3ncccn3)c2)n1)C1CCNCC1] | N-(piperidin-4-yl)-N-(propan- 2-yl)-2-[1-(pyrimidin-2-yl)-1H- pyrazol-4-yl]-1,3-thiazole-4- carboxamide |
| 330 | 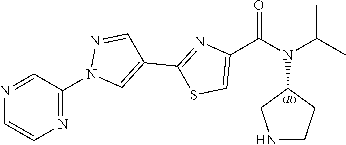 | N-(propan-2-yl)-2-[1- (pyrimidin-5-yl)-1H-pyrazol-4- yl]-N-[(3R)-pyrrolidin-3-yl]- 1,3-thiazole-4-carboxamide |
| 331 | 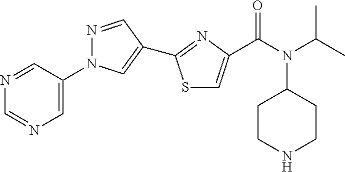 [SMILES: CC(C)N(C(=O)c1csc(-c2cnn(-c3cncnc3)c2)n1)C1CCNCC1] | N-(piperidin-4-yl)-N-(propan- 2-yl)-2-[1-(pyrimidin-5-yl)-1H- pyrazol-4-yl]-1,3-thiazole-4- carboxamide |
| 332 | 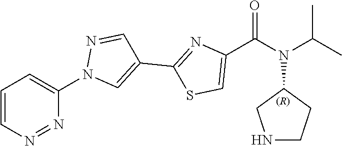 | N-(propan-2-yl)-2-[1- (pyridazin-3-yl)-1H-pyrazol-4- yl]-N-[(3R)-pyrrolidin-3-yl]- 1,3-thiazole-4-carboxamide |
| 333 | 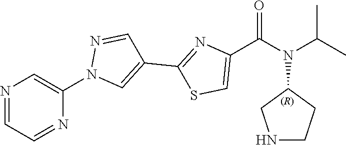 | N-(propan-2-yl)-2-[1-(pyrazin- 2-yl)-1H-pyrazol-4-yl]-N-[(3R)- pyrrolidin-3-yl]-1,3-thiazole-4-carboxamide |
| 334 | 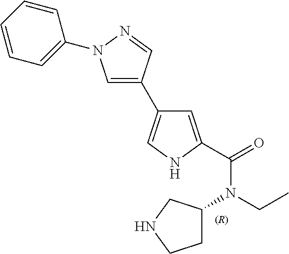 | 4-(1-phenyl-1H-pyrazol-4-yl)- N-(propan-2-yl)-N-[(3R)- pyrrolidin-3-yl]-1H-pyrrole-2- carboxamide |
| 335 | 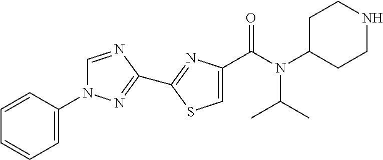 [SMILES: CC(C)N(C(=O)c1csc(-c2ncn(-c3ccccc3)n2)n1)C1CCNCC1] | 2-(1-phenyl-1H-1,2,4-triazol- 3-yl)-N-(piperidin-4-yl)-N- (propan-2-yl)-1,3-thiazole-4-carboxamide |
| 336 | 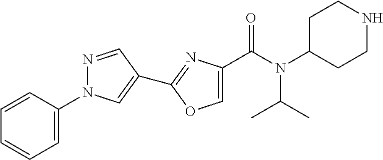 [SMILES: CC(C)N(C(=O)c1coc(-c2cnn(-c3ccccc3)c2)n1)C1CCNCC1] | 2-(1-phenyl-1H-pyrazol-4-yl)- N-(piperidin-4-yl)-N-(propan- 2-yl)-1,3-oxazole-4- carboxamide |
| 337 | 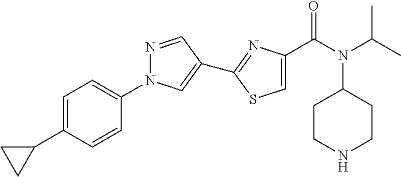 [SMILES: CC(C)N(C(=O)c1csc(-c2cnn(-c3ccc(C4CC4)cc3)c2)n1)C1CCNCC1] | 2-[1-(4-cyclopropylphenyl)- 1H-pyrazol-4-yl]-N-(piperidin- 4-yl)-N-(propan-2-yl)-1,3- thiazole-4-carboxamide |
| 338 | 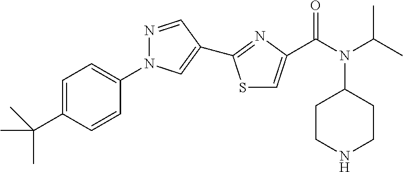 [SMILES: CC(C)N(C(=O)c1csc(-c2cnn(-c3ccc(C(C)(C)C)cc3)c2)n1)C1CCNCC1] | 2-[1-(4-tert-butylphenyl)-1H- pyrazol-4-yl]-N-(piperidin-4- yl)-N-(propan-2-yl)-1,3- thiazole-4-carboxamide |
| 339 | 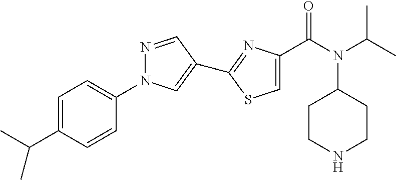 [SMILES: CC(C)c1ccc(-n2cc(-c3nc(C(=O)N(C(C)C)C4CCNCC4)cs3)cn2)cc1] | N-(piperidin-4-yl)-N-(propan- 2-yl)-2-{1-[4-(propan-2- yl)phenyl]-1H-pyrazol-4-yl}- 1,3-thiazole-4-carboxamide |
| 340 | 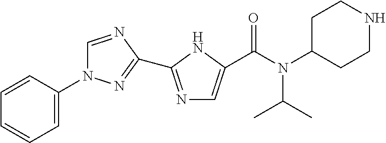 [SMILES: CC(C)N(C(=O)c1cnc(-c2ncn(-c3ccccc3)n2)[nH]1)C1CCNCC1] | 2-(1-phenyl-1H-pyrazol-4-yl)- N-(piperidin-4-yl)-N-(propan- 2-yl)-1H-imidazole-4- carboxamide |
| 341 | 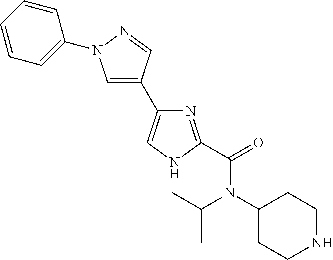 [SMILES: CC(C)N(C(=O)c1nc(-c2cnn(-c3ccccc3)c2)c[nH]1)C1CCNCC1] | 5-(1-phenyl-1H-pyrazol-4-yl)- N-(piperidin-4-yl)-N-(propan- 2-yl)-1H-imidazole-2- carboxamide |
| 342 | 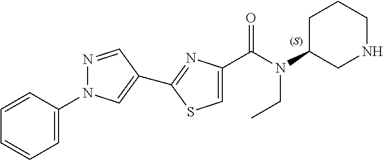 | N-ethyl-2-(1-phenyl-1H- pyrazol-4-yl)-N-[(3S)- piperidin-3-yl]-1,3-thiazole-4- carboxamide |
| 343 | 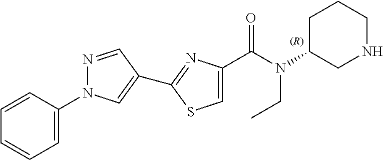 | N-ethyl-2-(1-phenyl-1H- pyrazol-4-yl)-N-[(3R)- piperidin-3-yl]-1,3-thiazole-4- carboxamide |
| 344 | 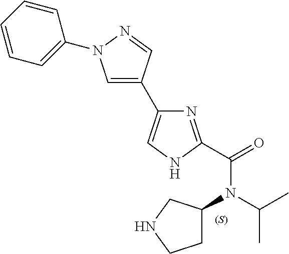 | 5-(1-phenyl-1H-pyrazol-4-yl)- N-(propan-2-yl)-N-[(3S)- pyrrolidin-3-yl]-1H-imidazole- 2-carboxamide |
| 345 | 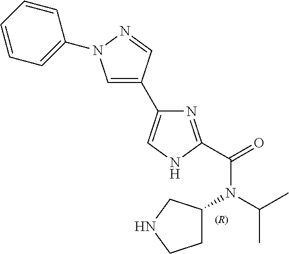 | 5-(1-phenyl-1H-pyrazol-4-yl)- N-(propan-2-yl)-N-[(3R)- pyrrolidin-3-yl]-1H-imidazole- 2-carboxamide |
| 346 | 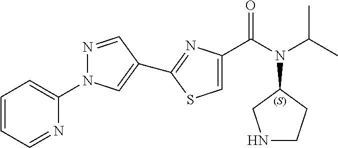 | N-(propan-2-yl)-2-[1-(pyridin- 2-yl)-1H-pyrazol-4-yl]-N-[(3S)- pyrrolidin-3-yl]-1,3-thiazole-4-carboxamide |
| 347 | 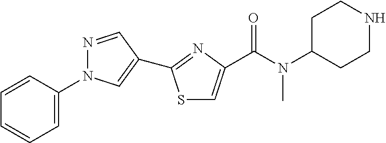 [SMILES: CN(C(=O)c1csc(-c2cnn(-c3ccccc3)c2)n1)C1CCNCC1] | N-methyl-2-(1-phenyl-1H- pyrazol-4-yl)-N-(piperidin-4- yl)-1,3-thiazole-4- carboxamide |
| 348 | 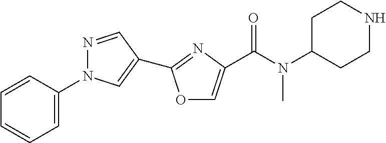 [SMILES: CN(C(=O)c1coc(-c2cnn(-c3ccccc3)c2)n1)C1CCNCC1] | N-methyl-2-(1-phenyl-1H- pyrazol-4-yl)-N-(piperidin-4- yl)-1,3-oxazole-4-carboxamide |
| 349 | 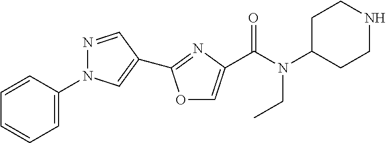 [SMILES: CCN(C(=O)c1coc(-c2cnn(-c3ccccc3)c2)n1)C1CCNCC1] | N-ethyl-2-(1-phenyl-1H- pyrazol-4-yl)-N-(piperidin-4- yl)-1,3-oxazole-4-carboxamide |
| 350 | 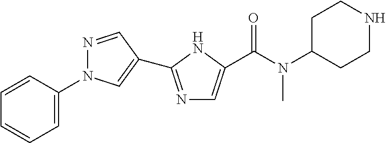 [SMILES: CN(C(=O)c1cnc(-c2cnn(-c3ccccc3)c2)[nH]1)C1CCNCC1] | N-methyl-2-(1-phenyl-1H- pyrazol-4-yl)-N-(piperidin-4- yl)-1H-imidazole-4- carboxamide |
| 351 | 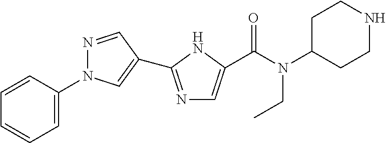 [SMILES: CCN(C(=O)c1cnc(-c2cnn(-c3ccccc3)c2)[nH]1)C1CCNCC1] | N-ethyl-2-(1-phenyl-1H- pyrazol-4-yl)-N-(piperidin-4- yl)-1H-imidazole-4- carboxamide |
| 352 | 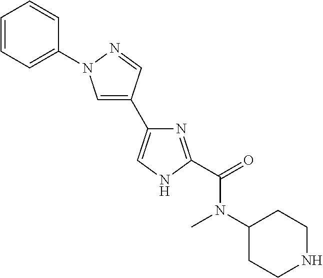 [SMILES: CN(C(=O)c1nc(-c2cnn(-c3ccccc3)c2)c[nH]1)C1CCNCC1] | N-methyl-5-(1-phenyl-1H- pyrazol-4-yl)-N-(piperidin-4- yl)-1H-imidazole-2- carboxamide |
| 353 | 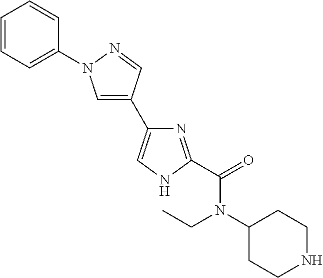 [SMILES: CCN(C(=O)c1nc(-c2cnn(-c3ccccc3)c2)c[nH]1)C1CCNCC1] | N-ethyl-5-(1-phenyl-1H- pyrazol-4-yl)-N-(piperidin-4- yl)-1H-imidazole-2- carboxamide |
| 354 | 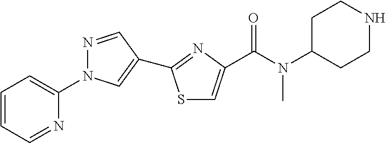 [SMILES: CN(C(=O)c1csc(-c2cnn(-c3ccccn3)c2)n1)C1CCNCC1] | N-methyl-N-(piperidin-4-yl)-2- [1-(pyridin-2-yl)-1H-pyrazol-4- yl]-1,3-thiazole-4- carboxamide |
| 355 | 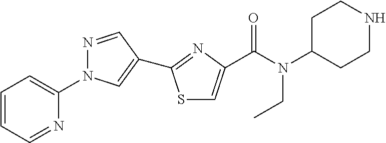 [SMILES: CCN(C(=O)c1csc(-c2cnn(-c3ccccn3)c2)n1)C1CCNCC1] | N-ethyl-N-(piperidin-4-yl)-2- [1-(pyridin-2-yl)-1H-pyrazol-4- yl]-1,3-thiazole-4- carboxamide |
| 356 | 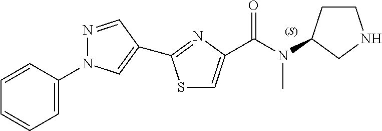 | N-methyl-2-(1-phenyl-1H- pyrazol-4-yl)-N-[(3S)- pyrrolidin-3-yl]-1,3-thiazole-4- carboxamide |
| 357 | 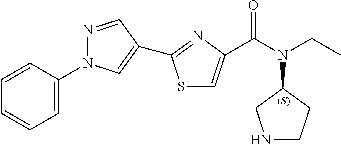 | N-ethyl-2-(1-phenyl-1H- pyrazol-4-yl)-N-[(3S)- pyrrolidin-3-yl]-1,3-thiazole-4- carboxamide |
| 358 | 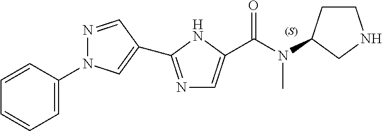 | N-methyl-2-(1-phenyl-1H- pyrazol-4-yl)-N-[(3S)- pyrrolidin-3-yl]-1H-imidazole- 4-carboxamide |
| 359 | 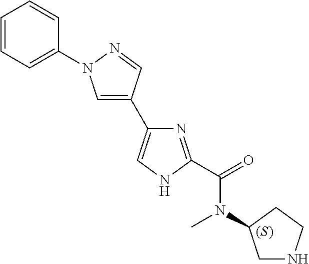 | N-methyl-5-(1-phenyl-1H- pyrazol-4-yl)-N-[(3S)- pyrrolidin-3-yl]-1H-imidazole- 2-carboxamide |
| 360 | 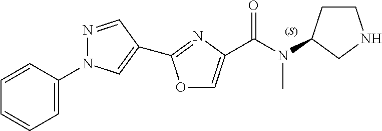 | N-methyl-2-(1-phenyl-1H- pyrazol-4-yl)-N-[(3S)- pyrrolidin-3-yl]-1,3-oxazole-4- carboxamide |
| 361 | 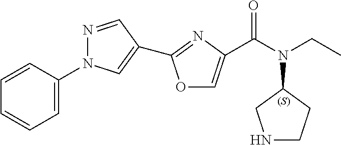 | N-ethyl-2-(1-phenyl-1H- pyrazol-4-yl)-N-[(3S)- pyrrolidin-3-yl]-1,3-oxazole-4- carboxamide |
| 362 | 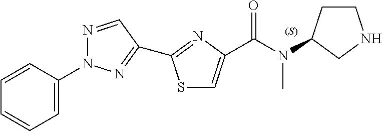 | N-methyl-2-(2-phenyl-2H- 1,2,3-triazol-4-yl)-N-[(3S)- pyrrolidin-3-yl]-1,3-thiazole-4-carboxamide |
| 363 | 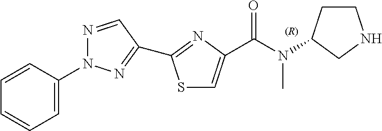 | N-methyl-2-(2-phenyl-2H- 1,2,3-triazol-4-yl)-N-[(3R)- pyrrolidin-3-yl]-1,3-thiazole-4-carboxamide |
| 364 | 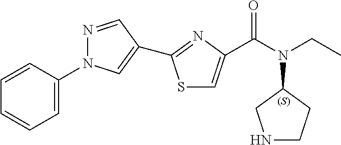 | N-ethyl-2-(2-phenyl-2H-1,2,3- triazol-4-yl)-N-[(3S)- pyrrolidin-3-yl]-1,3-thiazole-4- carboxamide |
| 365 | 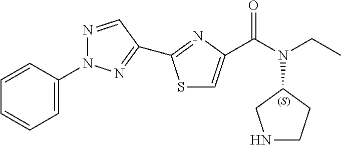 | N-ethyl-2-(2-phenyl-2H-1,2,3- triazol-4-yl)-N-[(3R)- pyrrolidin-3-yl]-1,3-thiazole-4- carboxamide |
| 366 | 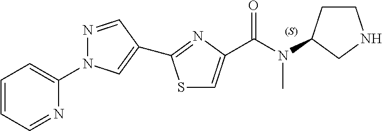 | N-methyl-2-[1-(pyridin-2-yl)- 1H-pyrazol-4-yl]-N-[(3S)- pyrrolidin-3-yl]-1,3-thiazole-4- carboxamide |
| 367 | 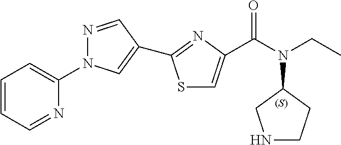 | N-ethyl-2-[1-(pyridin-2-yl)-1H- pyrazol-4-yl]-N-[(3S)- pyrrolidin-3-yl]-1,3-thiazole-4- carboxamide |
| 368 | 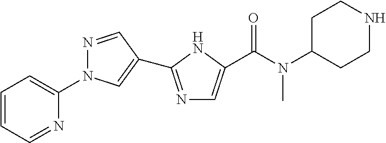 [SMILES: CN(C(=O)c1cnc(-c2cnn(-c3ccccn3)c2)[nH]1)C1CCNCC1] | N-methyl-N-(piperidin-4-yl)-2- [1-(pyridin-2-yl)-1H-pyrazol-4- yl]-1H-imidazole-4- carboxamide |
| 369 | 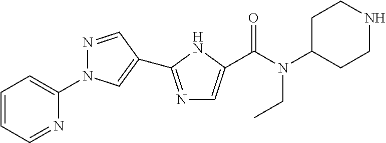 [SMILES: CCN(C(=O)c1cnc(-c2cnn(-c3ccccn3)c2)[nH]1)C1CCNCC1] | N-ethyl-N-(piperidin-4-yl)-2- [1-(pyridin-2-yl)-1H-pyrazol-4- yl]-1H-imidazole-4- carboxamide |
| 370 | 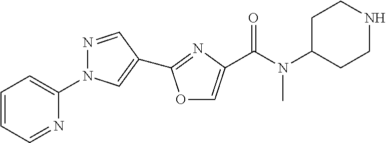 [SMILES: CN(C(=O)c1coc(-c2cnn(-c3ccccn3)c2)n1)C1CCNCC1] | N-methyl-N-(piperidin-4-yl)-2- [1-(pyridin-2-yl)-1H-pyrazol-4- yl]-1,3-oxazole-4- carboxamide |
| 371 | 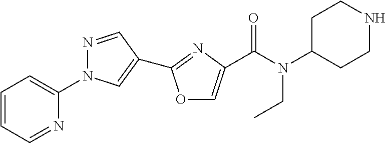 [SMILES: CCN(C(=O)c1coc(-c2cnn(-c3ccccn3)c2)n1)C1CCNCC1] | N-ethyl-N-(piperidin-4-yl)-2- [1-(pyridin-2-yl)-1H-pyrazol-4- yl]-1,3-oxazole-4- carboxamide |
| 372 | 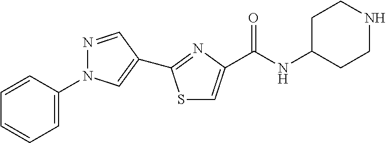 [SMILES: O=C(NC1CCNCC1)c1csc(-c2cnn(-c3ccccc3)c2)n1] | 2-(1-phenyl-1H-pyrazol-4-yl)- N-(piperidin-4-yl)-1,3- thiazole-4-carboxamide |
| 373 | 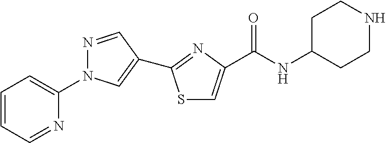 [SMILES: O=C(NC1CCNCC1)c1csc(-c2cnn(-c3ccccn3)c2)n1] | N-(piperidin-4-yl)-2-[1- (pyridin-2-yl)-1H-pyrazol-4- yl]-1,3-thiazole-4- carboxamide |
| 374 | 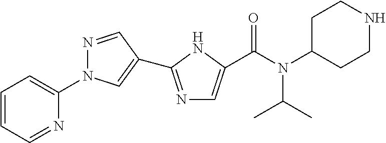 [SMILES: CC(C)N(C(=O)c1cnc(-c2cnn(-c3ccccn3)c2)[nH]1)C1CCNCC1] | N-(piperidin-4-yl)-N-(propan- 2-yl)-2-[1-(pyridin-2-yl)-1H- pyrazol-4-yl]-1H-imidazole-4- carboxamide |
| 375 | 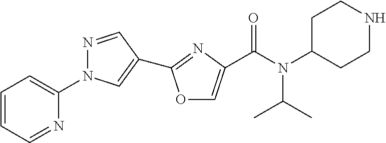 [SMILES: CC(C)N(C(=O)c1coc(-c2cnn(-c3ccccn3)c2)n1)C1CCNCC1] | N-(piperidin-4-yl)-N-(propan- 2-yl)-2-[1-(pyridin-2-yl)-1H- pyrazol-4-yl]-1,3-oxazole-4- carboxamide |
| 376 | 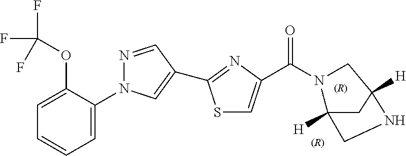 | (1R,4R)-2-(2-{1-[2- (trifluoromethoxy)phenyl]- 1H-pyrazol-4-yl}-1,3-thiazole- 4-carbonyl)-2,5- diazabicyclo[2.2.1]heptane |
| 377 | 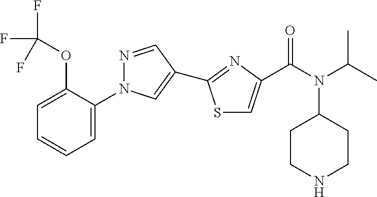 [SMILES: CC(C)N(C(=O)c1csc(-c2cnn(-c3ccccc3OC(F)(F)F)c2)n1)C1CCNCC1] | N-(piperidin-4-yl)-N-(propan-2-yl)-2-{1- [2-(trifluoromethoxy)phenyl]- 1H-pyrazol-4-yl}-1,3-thiazole- 4-carboxamide |
| 378 | 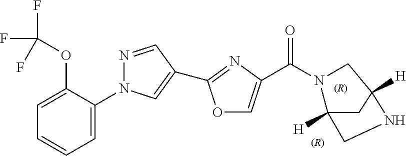 | (1R,4R)-2-(2-{1-[2- (trifluoromethoxy)phenyl]- 1H-pyrazol-4-yl}-1,3-oxazole- 4-carbonyl)-2,5- diazabicyclo[2.2.1]heptane |
| 379 | 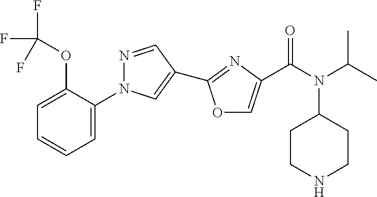 [SMILES: CC(C)N(C(=O)c1coc(-c2cnn(-c3ccccc3OC(F)(F)F)c2)n1)C1CCNCC1] | N-(piperidin-4-yl)-N-(propan- 2-yl)-2-{1-[2- (trifluoromethoxy)phenyl]- 1H-pyrazol-4-yl}-1,3-oxazole- 4-carboxamide |
| 380 | 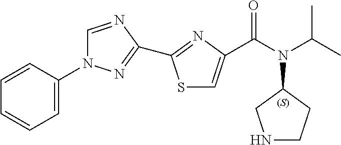 | 2-(1-phenyl-1H-1,2,4-triazol- 3-yl)-N-(propan-2-yl)-N-[(3S)- pyrrolidin-3-yl]-1,3-thiazole-4- carboxamide |
| 381 | 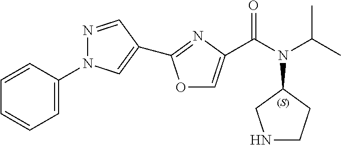 | 2-(1-phenyl-1H-pyrazol-4-yl)- N-(propan-2-yl)-N-[(3S)- pyrrolidin-3-yl]-1,3-oxazole-4- carboxamide |
| 382 | 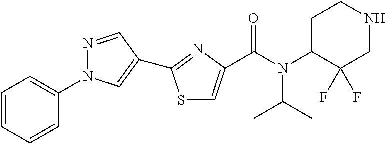 [SMILES: CC(C)N(C(=O)c1csc(-c2cnn(-c3ccccc3)c2)n1)C1CCNCC1(F)F] | N-(3,3-difluoropiperidin-4-yl)- 2-(1-phenyl-1H-pyrazol-4-yl)- N-(propan-2-yl)-1,3-thiazole- 4-carboxamide |
| 383 | 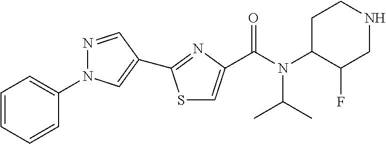 [SMILES: CC(C)N(C(=O)c1csc(-c2cnn(-c3ccccc3)c2)n1)C1CCNCC1F] | N-(3-fluoropiperidin-4-yl)-2- (1-phenyl-1H-pyrazol-4-yl)-N- (propan-2-yl)-1,3-thiazole-4- carboxamide |
| 384 | 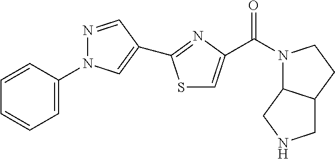 [SMILES: O=C(c1csc(-c2cnn(-c3ccccc3)c2)n1)N1CCC2CNCC21] | 4-{octahydropyrrolo[2,3- c]pyrrole-1-carbonyl}-2-(1- phenyl-1H-pyrazol-4-yl)-1,3- thiazole |
| 385 | 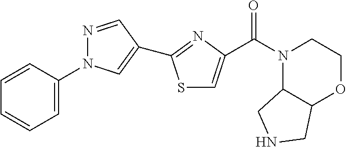 [SMILES: O=C(c1csc(-c2cnn(-c3ccccc3)c2)n1)N1CCOC2CNCC21] | 4-{octahydropyrrolo[3,4- b][1,4]oxazine-4-carbonyl}-2- (1-phenyl-1H-pyrazol-4-yl)- 1,3-thiazole |
| 386 | 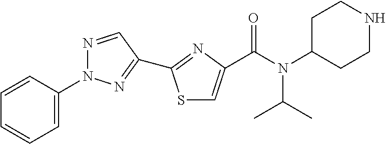 [SMILES: CC(C)N(C(=O)c1csc(-c2cnn(-c3ccccc3)n2)n1)C1CCNCC1] | 2-(2-phenyl-2H-1,2,3-triazol- 4-yl)-N-(piperidin-4-yl)-N- (propan-2-yl)-1,3-thiazole-4- carboxamide |
| 387 | 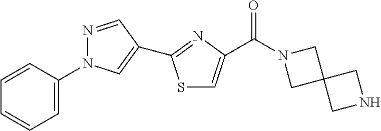 [SMILES: O=C(c1csc(-c2cnn(-c3ccccc3)c2)n1)N1CC2(CNC2)C1] | 2-[2-(1-phenyl-1H-pyrazol-4- yl)-1,3-thiazole-4-carbonyl]- 2,6-diazaspiro[3.3]heptane |
| 388 | 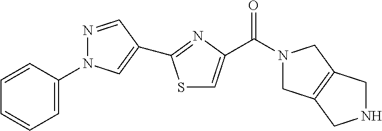 [SMILES: O=C(c1csc(-c2cnn(-c3ccccc3)c2)n1)N1CC2=C(CNC2)C1] | 2-(1-phenyl-1H-pyrazol-4-yl)- 4-{1H,2H,3H,4H,5H,6H- pyrrolo[3,4-c]pyrrole-2- carbonyl}-1,3-thiazole |
| 389 | 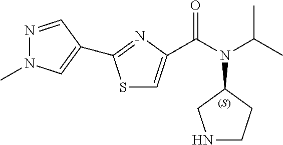 | 2-(1-methyl-1H-pyrazol-4-yl)- N-(propan-2-yl)-N-[(3S)- pyrrolidin-3-yl]-1,3-thiazole-4- carboxamide |
| 390 | 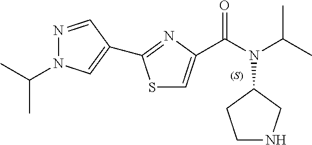 | N-(propan-2-yl)-2-[1-(propan- 2-yl)-1H-pyrazol-4-yl]-N-[(3S)- pyrrolidin-3-yl]-1,3-thiazole-4- carboxamide |
| 391 | 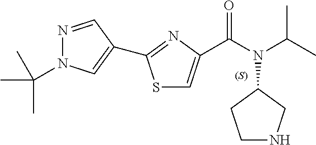 | 2-(1-tert-butyl-1H-pyrazol-4- yl)-N-(propan-2-yl)-N-[(3S)- pyrrolidin-3-yl]-1,3-thiazole-4- carboxamide |
| 392 | 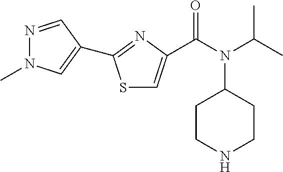 [SMILES: CC(C)N(C(=O)c1csc(-c2cnn(C)c2)n1)C1CCNCC1] | 2-(1-methyl-1H-pyrazol-4-yl)- N-(piperidin-4-yl)-N-(propan- 2-yl)-1,3-thiazole-4- carboxamide |
| 393 | 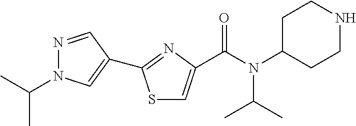 [SMILES: CC(C)N(C(=O)c1csc(-c2cnn(C(C)C)c2)n1)C1CCNCC1] | N-(piperidin-4-yl)-N-(propan- 2-yl)-2-[1-(propan-2-yl)-1H- pyrazol-4-yl]-1,3-thiazole-4- carboxamide |
| 394 | 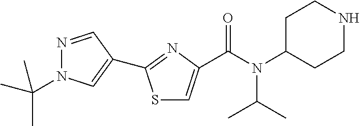 [SMILES: CC(C)N(C(=O)c1csc(-c2cnn(C(C)(C)C)c2)n1)C1CCNCC1] | 2-(1-tert-butyl-1H-pyrazol-4- yl)-N-(piperidin-4-yl)-N- (propan-2-yl)-1,3-thiazole-4- carboxamide |
| 395 | 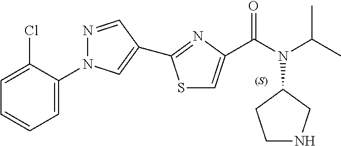 | 2-[1-(2-chlorophenyl)-1H- pyrazol-4-yl]-N-(propan-2-yl)- N-[(3S)-pyrrolidin-3-yl]-1,3- thiazole-4-carboxamide |
| 396 | 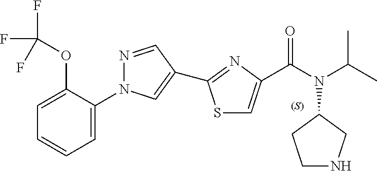 | N-(propan-2-yl)-N-[(3S)- pyrrolidin-3-yl]-2-{1-[2- (trifluoromethoxy)phenyl]- 1H-pyrazol-4-yl}-1,3-thiazole- 4-carboxamide |
| 397 | 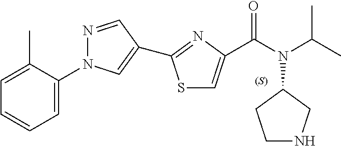 | 2-[1-(2-methylphenyl)-1H- pyrazol-4-yl]-N-(propan-2-yl)- N-[(3S)-pyrrolidin-3-yl]-1,3- thiazole-4-carboxamide |
| 398 | 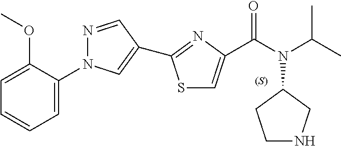 | 2-[1-(2-methoxyphenyl)-1H- pyrazol-4-yl]-N-(propan-2-yl)- N-[(3S)-pyrrolidin-3-yl]-1,3- thiazole-4-carboxamide |

### Biological Assays and Data

As stated above, the compounds of the present invention induce and/or stimulate autophagy and are useful in treating autophagy-related diseases. The biological activity of the compounds of the present invention can be determined by any appropriate test to determine the ability to induce and/or stimulate autophagy.

### Assessment of Stimulation of Autophagy

Compounds of this invention were assessed for their ability to stimulate autophagy using one, or more, of the assays described below

### Assay to Measure Lysosome and Autolysosome Formation

Lysosomes play a fundamental role in the autophagic pathway by fusing with autophagosomes and creating ‘autolysosomes’ in order to digest their contents. Stimulation of lysosome and autolysosome formation by a compound is indicative of a stimulation of autophagy. The ability of compounds to stimulate lysosome and autolysosome formation, and thus autophagy, in live cells was assessed via fluorescent microscopy using various fluorescent stains for labelling and tracking acidic organelles (including lysosomes and autolysosomes) such as: LysoView™ 650 (70059 and 70059-T, Biotium), LysoViewTM633 (70058 and 70058-T, Biotium) and LysoTracker™ Deep Red (L12492, ThermoFisher Scientific). The cellular phenotype was quantitatively assessed for the induction of acidic vesicle formation and compared to a non-treated control, thus providing a measure of the ability of the compound under investigation to stimulate autophagy.

Representative procedure using LysoView™633 dye or LysoTracker™ Deep Red dye: Human osteosarcoma U20S cells (40,000 cells/well) were seeded in a 24 well glass bottom plate (Sensoplate, Greiner Bio-One) and were incubated overnight in a humidified atmosphere at 37° C. and 5% CO2. Cells were grown in DMEM (Gibco) supplemented with 10% Fetal Bovine Serum (FBS) and 100 units/ml penicillin and 100 μg/ml streptomycin (Invitrogen). After the attachment period, cells were treated with different compounds of interest (at various concentrations in DMSO) or DMSO (non-treated control) in cell culture medium and were incubated for 24 hours. Compound-containing medium was removed and cells were incubated with pre-warmed cell culture medium containing 1× LysoView™ 633 (70058 and 70058-T, Biotium) or 50 nM LysoTracker™ Deep Red (L12492, ThermoFisher Scientific) for 45 minutes at 37° C. Finally, cell nuclei were stained for 10 minutes using Hoechst 33342 (1 μg/mL) and then the medium was replaced with fresh medium. The 24 well plate was then fitted into a heated stage on the microscope and cells maintained at 37° C. Images were captured using appropriate filter set for Cy5 and DAPI detection with the EVOS M7000 Microscope (ThermoFisher Scientific). The cellular phenotype was visually assessed for the induction of acidic vesicle formation relative to the non-treated control; multiple images were acquired and analyzed using ImageJ or Cell Profiler.

All of the compounds according to the invention, as well as the preferred racemic mixture of compounds 170 and 171 showed clearly increased acidic vesicle formation in human osteosarcoma U20S cells relative to a DMSO-treated control (non-treated control) at a concentration of 10 μM except compounds 317, 322 and 332, which showed only a slight increase. In this assay, acidic vesicle formation was increased by at least 25% for all compounds including the racemic mixture of compounds 170 and 171 except compounds 310, 317, 320, 321, 322, 323, 329, 332 and 334, which showed only a slight increase. This is evidence for a significant stimulation of autophagy by these compounds under the conditions as tested.

Selected compounds of this invention were also tested and showed increased acidic vesicle formation in human osteosarcoma U2OS cells relative to a DMSO-treated control (non-treated control) at a concentration of 0.8 μM. This is evidence for significant stimulation of autophagy by the compounds of this invention, and particularly by these compounds under the conditions as tested. Table 2 shows this data for the selected compounds. Compounds having an activity designated as “+” provided a percentage increase in acidic vesicle formation of between 10% and 30% over the DMSO-treated control. Compounds having an activity designated as “++” provided a percentage increase in acidic vesicle formation of between 30% and 50% over the DMSO-treated control. Compounds having an activity designated as “+++” provided a percentage increase in acidic vesicle formation of between 50% and 100% over the DMSO-treated control. Compounds having an activity designated as “++++” provided a percentage increase in acidic vesicle of greater than 100% over the DMSO-treated control. Preferred compounds are shown in Table 2a, more preferred compounds are shown in Table 2b.

TABLE 2

| Compound | |
|---|---|
| No. | Activity |
| 5 | ++ |
| 11 | + |
| 16 | + |
| 17 | + |
| 23 | + |
| 25 | + |
| 26 | ++ |
| 46 | ++ |
| 28 | + |
| 29 | ++ |
| 31 | + |
| 35 | + |
| 36 | + |
| 40 | + |
| 41 | + |
| 42 | + |
| 43 | + |
| 44 | + |
| 45 | ++ |
| 47 | ++ |
| 48 | +++ |
| 49 | ++ |
| 50 | +++ |
| 51 | ++ |
| 52 | + |
| 53 | + |
| 54 | + |
| 76 | + |
| 56 | + |
| 57 | ++ |
| 58 | + |
| 59 | + |
| 65 | ++ |
| 66 | + |
| 69 | ++ |
| 70 | ++ |
| 94 | ++++ |
| 95 | +++ |
| 97 | ++ |
| 82 | ++ |
| 83 | ++ |
| 84 | + |
| 86 | ++ |
| 87 | + |
| 89 | + |
| 90 | + |
| 92 | +++ |
| 93 | ++++ |
| 98 | +++ |
| 99 | +++ |
| 100 | ++ |
| 101 | ++ |
| 104 | + |
| 107 | + |
| 110 | +++ |
| 111 | ++ |
| 116 | +++ |
| 120 | +++ |
| 122 | ++++ |
| 123 | ++++ |
| 124 | +++ |
| 130 | + |
| 140 | + |
| 141 | +++ |
| 142 | + |
| 143 | + |
| 153 | + |
| 159 | ++ |
| 162 | + |
| 163 | + |
| 167 | + |
| 170 | + |
| 171 | + |
| 177 | + |
| 179 | ++ |
| 180 | ++ |
| 183 | + |
| 316 | ++ |
| 319 | ++ |
| 321 | ++ |
| 323 | ++ |
| 327 | ++ |
| 334 | ++ |
| 307 | +++ |
| 311 | +++ |
| 312 | +++ |
| 326 | +++ |
| 313 | ++++ |
| 314 | ++++ |
| 318 | ++++ |

TABLE 2a
Preferred compounds

| Compound | |
|---|---|
| No. | Activity |
| 110 | +++ |
| 116 | +++ |
| 120 | +++ |
| 122 | +++ |
| 123 | +++ |
| 124 | +++ |
| 141 | +++ |
| 48 | +++ |
| 50 | +++ |
| 92 | +++ |
| 93 | +++ |
| 94 | +++ |
| 95 | +++ |
| 98 | +++ |
| 99 | +++ |

TABLE 2b
More preferred compounds

| Compound No. | Activity |
|---|---|
| 122 | ++++ |
| 123 | ++++ |
| 93 | ++++ |
| 94 | ++++ |

Certain compounds of this invention also showed increased acidic vesicle formation in other cell types, relative to a DMSO-treated control (non-treated control). For example, the racemic mixture of compounds 170 and 171 showed significantly greater acidic vesicle formation in adult human dermal fibroblast cells (HDFa cells) relative to a DMSO-treated control at a concentration of 10 μM. This is evidence for significant stimulation of autophagy by this compound under these conditions.

In another example, certain compounds of this invention were found to show increased acidic vesicle formation relative to a DMSO-treated control (non-treated control) in SH-SY5Y cells (Table 3). This is evidence for significant stimulation of autophagy by this compound under these conditions. A brief description of the procedure is outlined here:
Day 1: Cells were seeded at 15K cells/well in the middle 60 wells of 6×96 well Cellvis imaging plates
Day 2: Media was replaced with 90 μl of DMEM/F12 (+10% FBS/1% P/S) containing NucBlue Live and the cells were treated with 10 μl of compound (10× concentrated) in triplicate for 24 hours.
Day3: Treatment was removed and cells were incubated for 30 minutes with LysoTracker™ Deep Red dye. Cells were washed twice with media and immediately imaged on EVOS M7000 at x20 (6 images per well, 30 minutes imaging time/plate). Images were analysed using Celleste Image analysis software.

TABLE 3
Fold-change in acidic vesicle formation relative
to DMSO-treated control (non-treated control)
in SH-SY5Y cells

| | Fold change at | Fold change at |
|---|---|---|
| | compound | compound |
| | concentration | concentration |
| Compound | of 0.8 μM | of 10 μM |
| 311 | 2.3 | 3.5 |
| 313 | 2.0 | 13.2 |
| 314 | 2.0 | 14.3 |
| 320 | 1.9 | 3.3 |
| 310 | 1.9 | 4.5 |
| 319 | 2.4 | 2.7 |

### Tandem Reporter Assay (Assay to Assess Autophagic Flux)

Selected compounds of this invention were also assessed for their ability to stimulate autophagy using a U20S cell line stabling expressing RFP-eGFP-hLC3b (a tandem reporter cell line). During the process of autophagy, the cargo to be degraded is first enveloped by organelles called autophagosomes. These autophagosomes then fuse with lysosomes, causing them to become acidified and their digestive enzymes to become activated. The term “autophagic flux” is used to represent the dynamic process of autophagy: autophagic flux refers to the whole process of autophagy, including autophagosome formation, maturation, fusion with lysosomes, subsequent breakdown and the release of macromolecules back into the cytosol (Zhang X J, Chen S, Huang K X, Le W D. Why should autophagic flux be assessed? Acta Pharmacol Sin. 2013 May; 34(5): 595-9. doi: 10.1038/aps.2012.184. Epub 2013 Mar. 11). Compounds that stimulate autophagic flux stimulate the dynamic process of autophagy (the whole process of autophagy).

LC3b is a protein found in the membrane of autophagosomes which has been used to generate genetic reporters of autophagy in cells. In these systems a tandem fusion of LC3b is engineered with two fluorescent proteins of different wavelengths: one which is acid sensitive (for example eGFP, which fluoresces green) and the other which is acid insensitive (for example RFP, which fluoresces red). Cells expressing RFP-eGFP-hLC3b can be examined using fluorescent microscopy: the autophagosomes present will fluoresce in both channels (either yellow in an overlay of both channels or puncta that are present in both the individual red and green channels) but the autolysosomes present will fluoresce in the RFP (red) channel only. Using a fluorescent microscope, the number of autophagosomes and autolysosomes can be counted, allowing for the monitoring of both the induction of autophagy (total puncta count) and the rate of autophagic flux (ratio of red-only to red-and-green puncta). The influence of small molecules on autophagy induction and flux can be assessed using this system by comparison to a non-treated control.

Representative Procedure: Human osteosarcoma U2OS cells stably expressing RFP-eGFP-hLC3b were seeded (10,000 cells/well) into a 96-well glass-bottom plate (Cell Carrier Ultra, Perkin Elmer) in DMEM Glutamax media (Gibco) supplemented with 10% Fetal Bovine Serum (FBS) and antibiotics (100 u/ml penicillin, 100 μg/ml streptomycin, Invitrogen), then incubated overnight in a humified atmosphere at 37° C. and 5% CO2. The cells were then treated with the compounds of interest, dissolved in DMSO, in a duplicated 5- or 10-point dose-response. 8 wells were treated with an equivalent volume of DMSO, 4 with 0.3 μM torin-1 as a positive control (MCE) and 4 wells with 0.4 μM torin-1 plus 0.2 μM bafilomycin (MCE) as a control for flux inhibition. The cells were returned to the incubator and the treatment continued for 24 hours. After treatment, the media was aspirated off and the cells fixed with a solution of 4% formaldehyde+1% glutaraldehyde (Sigma) in dPBS (plus magnesium and calcium, Gibco).

Fixation was allowed to proceed for 15 minutes at room temperature then the fixative solution discarded and replaced with PBS (Gibco) plus Hoescht 33342 (1 g/ml, Sigma). After 30 minutes the plates were imaged on an Opera Phenix confocal microscope (Perkin-Elmer) using a 40× water objective, collecting using DAPI, mCherry and GFP channels. Cells were detected based upon the staining of their nuclei with DAPI using the Harmony image analysis software (Perkin-Elmer) and the number of autophagosomes (GFP and RFP puncta) and autolysosomes (RFP only puncta) per cell were counted using the spot picking function within the software.

Selected compounds of this invention showed an increase in the number of RFP-only puncta (autolysosomes) relative to a DMSO-treated control (non-treated control) at different concentrations (Table 4). This is evidence for significant stimulation of autophagic flux (and thus the whole process of autophagy) by the compounds of this invention, and particularly by these compounds under the conditions as tested.

TABLE 4
Change in the number of RFP-only puncta (autolysosomes)
relative to a DMSO-treated control (non-treated control)
in a U2OS cell line stabling expressing RFP-eGFP-hLC3b
(a tandem reporter cell line) “Autolysosome % DMSO activity”
is the activity of the compound (based on the number of RFP-only
puncta ((autolysosomes)) as a percentage of the activity of the
DMSO-treated control (non-treated control).

| | Autolysosome % DMSO | Autolysosome % DMSO |
|---|---|---|
| | activity (compound | activity (compound |
| Compound | concentration of 10 μM) | concentration of 1 μM)) |
| 163 | 187% (average of four runs) | 144% (average of four runs) |
| 171 | 294% (average of two runs) | 200% (average of two runs) |
| 398 | 190 | 116 |
| 397 | 191 | 115 |
| 396 | 78 | 138 |
| 395 | 191 | 137 |
| 394 | 176 | 114 |
| 393 | 110 | 106 |
| 392 | 109 | 97 |
| 391 | 161 | 109 |
| 390 | 116 | 106 |
| 389 | 108 | 101 |
| 382 | 108 | 96 |
| 345 | 72 | 116 |
| 383 | 194 | 98 |
| 359 | 144 | 113 |
| 381 | 179 | 118 |
| 378 | 199 | 122 |
| 365 | — | 183 |
| 376 | 260 | 176 |
| 364 | — | 166 |
| 367 | 215 | 114 |
| 363 | — | 171 |
| 360 | 214 | 113 |
| 358 | 103 | 102 |

Based on these results this is evidence that these compounds induce autophagy. Additional evidence that this activity will be therapeutic in various autophagy-related diseases are disclosed in the following examples.

### Assessment of Activity in Models of Various Diseases and Conditions

### Example 1

Demonstration of Activity in an Induced Pluripotent Stem Cell (iPSQ) Model of Glycogen Storage Disease Type 1A (GSD1A) Derived from Human Patient Cells.

The racemic mixture of compounds 170 and 171 was selected as an example compound of this invention to be evaluated in this model.

Timetable for Treatment of Cells with the Compounds:
Day 1. Differentiated hepatocyte GSD1A disease model cells (DefiniGEN Ltd) were cultured in a collagen I coated 48-well plate. Empty wells were filled with PBS.
Day 9. The racemic mixture of compounds 170 and 171 (as a solution in DMSO) was added to the disease model cells
Day 11. Media for disease model cells was replaced with fresh media with compound.
Day 12. Cells were washed once in PBS and once in HBSS and 30 μL of NP40 Lysis buffer supplied with Phosphatase and Protease inhibitor cocktail was added to each well. Plate was immediately placed on dry ice.

Lysate preparation: Cell lysates were thawed and 30 μL of RIPA buffer supplied with Phosphatase and Protease inhibitor cocktail was added to each.

Protein quantification: An aliquot of the lysates was used for protein quantification using BCA.

Glycogen quantification: An aliquot of the lysates was boiled at 96° C. for 5 minutes. Boiled lysates were then centrifuged at 1000×g for 5 minutes to remove precipitates. Supernatant was used for Glycogen quantification using MAK016 kit (Merck), following manufacturer's instructions. Briefly, in 2 aliquots 2 μL of each supernatant was transferred to a half-area 96-well plate. 8 μL of Hydrolysis buffer from the kit was added to one aliquot from each sample (-HE; to quantify free glucose content of the lysate). The other sample was supplied with 8 μL of Hydrolysis buffer supplied with Hydrolysis Enzyme (+HE; to quantify free glucose+glycogen content of the lysate). After 30 minutes of incubation at room temperature, 10 μL of probe mix was added to each well and the absorbance at 570 nm was measured after 30 minutes incubation at room temperature. A570 readings were first adjusted based on the blank readings. Glycogen content of the cells was estimated by subtracting −HE readings from +HE readings, and the values were normalised to DMSO only control. The quantification experiment was performed twice.
Triglyceride quantification: Boiled supernatants from the previous experiment were also used for Triglyceride quantification using MAK266 kit (Merck), following manufacturer's instructions. Briefly, 2 μL of each supernatant was transferred to a half-area 96-well plate. 8 μl of Lipase reaction mix from the kit was added each sample. After 20 minutes of incubation at room temperature, 10 μL of probe mix was added to each well and the absorbance at 570 nm was measured after 30 minutes incubation at room temperature. A570 readings were first adjusted based on the blank readings and the values were normalised to DMSO only control. Note that the quantification experiment was performed twice.

Results: The racemic mixture of compounds 170 and 171 at a concentration of 10 μM successfully reduced cellular glycogen and triglycerides in hepatocytes differentiated from GSD1A patient derived iPSCs (by over 75% and 20%, respectively) when compared with the DMSO-only control.

Conclusion: The demonstrated breakdown of glycogen in patient-derived differentiated hepatocytes strongly supports efficacy of compounds in this invention in the treatment of GSD1A. The breakdown of glycogen is a specific type of autophagy known as glycophagy.

### Example 2

### Demonstration of Antiviral Activity Against Human Cytomegalovirus (HCMV) and Zika Virus (ZIKV)

The racemic mixture of compounds 170 and 171 was selected as an example compound of this invention to be evaluated in this model.

### Infective Assay:

Cells were seeded in 96 well plates and the following day pre-treated with a 3-fold serial dilution of compound starting from 20 μM, in 100 μL of assay media. 24 hours later, cells were infected (without removing the media) with ZIKV or HCMV at a multiplicity of infection (MOI) compatible with multiple rounds of virus replication. DMSO-treated infected and uninfected cells were included as positive and negative controls. The experimental conditions for each virus are summarized in Table 5.

TABLE 5
Experimental conditions used in the infectivity assays.

| | | Assay | | Assay | Stained |
|---|---|---|---|---|---|
| Virus | Cell line | media | MOI | duration | antigen |
| ZIKV | antigenSH- | DMEM/F12 | 5 | 48 h | E |
| | SY5Y | 2% FBS | | | |
| HCMV | HFF | DMEM | 1 | 15 days | gB |
| | | 5% FBS | | | |

FBS: foetal bovine serum, E: envelope, gB: glycoprotein B.
MOI: multiplicity of infection (number of infectious viruses per cell).
At the indicated time points, selected to allow for more than one round of virus replication, the cells were fixed and immuno-stained for virus specific antigens.
Images were acquired on a Perkin Elmer Opera LTX and the percentage of infected cells was calculated using Columbus software.

### Cytotoxicity Assay:

In parallel to the infectivity assays, in order to determine the cytotoxic properties of the compound, cells were seeded in 96 well plates and the following day pre-treated with the same serial dilutions of compound in 100 μl of assay media. The compound was left on the cells for the duration of the infectivity assays, after which a metabolic assay (MTT assay) was used to determine cell viability compared to a DMSO-treated control.

Results: The IC50 and TC50 values of compound 170/171 for ZIKV and HCMV are summarized in Table 6.

TABLE 6

| | HCMV | | ZIKV | |
|---|---|---|---|---|
| | IC50 | TC50 | IC50 | TC50 |
| The racemic | <10 | >75 | <15 | — |
| mixture of | | | | |
| compounds | | | | |
| 170 and 171 | | | | |

IC50 and TC50 values for the compound according to formula 170/171 against HCMV and ZIKV.
IC50 and TC50 are reported in micromolar.
A “—” symbol indicates no inhibition.

Conclusion: The racemic mixture of compounds 170 and 171 was found to demonstrate antiviral activity against both HCMV and ZIKV over a range of concentrations less than 20 μM (IC50<10 μM and <15 μM respectively) with a limited toxicity profile. This is evidence that compounds of this invention are efficacious in the treatment of certain autophagy-related viral infections

### Example 3

The racemic mixture of compounds 170 and 171 was selected as an example compound of this invention to be evaluated in this model.

### Activity in a Cellular Model of NAFLD: Gel Assay for Mutant PNPLA3-Expressing HepG2 Cells

Method: HepG2 cells were transfected with GFP-PNPLA3 (1148>M) overexpression plasmid using Lipofectamine LTX in DMEM media supplied with 1% Oleic Acid-BSA and were left overnight. The next day, cells were trypsinated and were seeded in a 96-well plate in DMEM media supplied with 1% Oleic Acid-BSA. After 7 hours, media was changed to standard DMEM supplied with the compound of interest. After 24 hours, cells were washed once with PBS and were lysed in RIPA buffer. Lysates were collected, centrifuged and the supernatants were mixed with Laemmli buffer (without DTT or boiling) and were loaded on a 4-20% SDS-PAGE gel. After electrophoresis, gels were directly imaged in the FITC channel to visualise the GFP signal.

Results: The racemic mixture of compounds 170 and 171 showed a 3.7 fold increase in GFP breakdown Bafilomycin-sensitive-product signal versus an inactive control. Therefore, the racemic mixture of compounds 170 and 171 is responsible for lysosomal breakdown of mutated PNPLA3 in this model.

The 1148>M mutated form of PNPLA3 is associated with increased risk of Non-Alcohol Fatty Liver Disease [NAFLD]. Therefore, this model functions as an in vitro model of NAFLD, and it is an example of conditions and diseases relating to degradation of misfolded proteins. Activity in this model is also evidence that compounds of this invention are active versus various diseases involving misfolded proteins.

### Example 4

### Activity in a Cellular Model of Parkinson's Disease

Compounds 156 and 171 were selected as example compounds of this invention to be evaluated in this model.

This model (run by Neuro-Sys SAS, France) is based on a primary culture of dopaminergic Tyrosine hydroxylase (TH)-positive neurons injured with the dopaminergic neurons-specific toxin (DA-toxin) 1-methyl-4-phenyl-1,2,3,6 tetrahydropyridine (MPTP, a prodrug of MPP*) (Dauer and Przedborski, 2003). Any substances reducing DA-toxin neurotoxicity may be useful as a new therapeutic agent for the treatment or prevention of PD.

### Methods

Primary culture of mesencephalic neurons: Rat dopaminergic neurons were cultured as described by Visanji et al., 2008 and Callizot et al., 2019. Briefly, pregnant female rat (Wistar) of 15 days of gestation were killed using a deep anesthesia with CO2 chamber and a cervical dislocation. The midbrains obtained from 15-day-old rat embryos (Janvier, France) were dissected under a microscope. The embryonic midbrains will be removed and placed in ice-cold medium of Leibovitz (L15) containing 2% of Penicillin-Streptomycin (PS) and 1% of bovine serum albumin (BSA). The ventral portion of the mesencephalic flexure, a region of the developing brain rich in dopaminergic neurons, was used for the cell preparations.

The midbrains were dissociated by trypsinisation for 20 min at 37° C. (solution at a final concentration of 0.05% trypsin and 0.02% EDTA). The reaction was stopped by the addition of Dulbecco's modified Eagle's medium (DMEM) containing DNAase I grade II (0.5 mg/mL) and 10% of foetal calf serum (FCS). Cells were then mechanically dissociated by 3 passages through a 10 ml pipette. Cells were then centrifuged at 180×g for 10 min at +4° C. on a layer of BSA (3.5%) in L15 medium. The supernatant was discarded and the cell pellets re-suspended in a defined culture medium consisting of Neurobasal supplemented with B27 (2%), L-glutamine (2 mM) and 2% of PS solution and 10 ng/mL of Brain-derived neurotrophic factor (BDNF) and 1 ng/mL of Glial-Derived Neurotrophic Factor (GDNF). Viable cells were counted in a Neubauer cytometer using the trypan blue exclusion test. The cells were seeded at a density of 40,000 cells/well in 96 well-plates (pre-coated with poly-L-lysine) and maintained in a humidified incubator at 37° C. in 5% CO2/95% air atmosphere. Half of the medium was changed every 2 days with fresh medium. The wells of first and last lines and columns were used (to avoid any edge effect) and were be filled with sterile water.

Test compounds and MPTP:
i) Pre-incubation. On day 6 of culture, the test compounds were dissolved in PBS or DMSO and incubated for 1 hour before the MPTP exposure.
ii) Injury. One hour after the application of test compound, MPTP was added to a final concentration of 4 μM, diluted in control medium still in presence of compounds/inhibitors for 48h. Test compounds were tested on one culture in 96-well plate (n=6 culture wells per condition).

### Endpoint Evaluation:

Immunostaining: TH and α-Synuclein. 48 Hours after intoxication, the cell culture supernatant was removed, and the cells were fixed by a solution of 4% paraformaldehyde in PBS, pH=7.3 for 20 min at room temperature. The cells were washed twice in PBS, and then permeabilized. Non-specific sites were blocked with a solution of PBS containing 0.1% of saponin and 1% FCS for 15 min at room temperature. The cultures were incubated with: (a) monoclonal anti-Tyrosine Hydroxylase (TH) antibody produced in mouse at dilution of 1/10000 in PBS containing 1% FCS, 0.1% saponin, for 2 hours at room temperature, and (b) polyclonal anti-alpha synuclein (α-synuclein) antibody produced in rabbit at dilution of 1/200 in PBS containing 1% FCS, 0.1% saponin, for 2 h at room temperature. These antibodies were revealed with Alexa Fluor 488 goat anti-mouse IgG at the dilution 1/800 and with Alexa Fluor 568 goat anti-rabbit IgG at the dilution 1/400 in PBS containing 1% FCS, 0.1% saponin, for 1 h at room temperature.

Automatic computer analysis. For each condition, 20 pictures (representing the whole well area) were automatically taken using ImageXpress® (Molecular Devices) at 10× magnification (20 pictures, for TH and α-synuclein into TH neurons) using the same acquisition parameters. From images, analyses was directly and automatically performed by MetaXpress® (Molecular Devices).

The Following Read-Outs were Measured:
i) Analysis of total number of TH neurons (dopaminergic TH positive neurons)
ii) Total neurite network of dopaminergic TH positive neurons (in m)
iii) α-Synuclein aggregation (overlapping between TH and α-syn staining) Compounds 156 and 171 showed positive effects in this in vitro model of PD based on MPP+ injury.

Compound 156 significantly protected the dopaminergic neurons at concentrations of 10 μM, 1 μM and 500 nM (>18% increase in the number of dopaminergic TH positive neurons compared to MPP+ injury alone) and protected the neurite network at concentrations of 10 μM, 1 μM and 500 nM (>39% increase in the length of the neurite network of dopaminergic TH positive neurons compared to the MPP+ injury alone). This compound lowered α-Synuclein aggregation at concentrations of 10 μM, 1 μM, 500 nM and 100 nM (>13% decrease in α-syn area per dopaminergic TH positive neuron compared to MPP+ injury alone).

Compound 171 showed significant positive effects at concentrations of 500 μM and 1 μM on all three read-outs: (i) a >25% increase in the number of dopaminergic TH positive neurons compared to MPP+ injury alone, (ii) a >31% increase in the length of the neurite network of dopaminergic TH positive neurons compared to the MPP+ injury alone, and (iii) a >18% decrease in α-syn area per dopaminergic TH positive neuron compared to MPP+ injury alone.

This assay is an in vitro model of Parkinson's disease. Activity in this model (positive effects) strongly support that compounds of this invention are efficacious in the treatment of Parkinson's disease and other autophagy-related neurodegenerative diseases or conditions.

### REFERENCES

Callizot N, Combes M, Henriques A, Poindron P. Necrosis, apoptosis, necroptosis, three modes of action of dopaminergic neuron neurotoxins. PLoS ONE 14(4): e0215277
Dauer W., Przedborski S. Parkinson's disease: mechanisms and models. Neuron 2003, 39(6):889-909
Visanji N P, Orsi A, Johnston T H, Howson P A, Dixon K, Callizot N, Brotchie J M and Rees D D. PYM50028, a novel, orally active, nonpeptide neurotrophic factor inducer, prevents and reverses neuronal damage induced by MPP+ in mesencephalic neurons and by MPTP in a mouse model of Parkinson's disease. FASEB J. 2008; 22(7):2488-97

### Example 5

### Activity in a Mouse Model of Charcot-Marie-Tooth 1A (CMT1A)

Compound 163 was selected as an example compound of this invention to be evaluated in this model. This model (run by In vivex SAS, France) uses C3-PMP22 transgenic mice (B6·Cg-Tg(PMP22)C3Fbas/J) which express three copies of a wild-type human peripheral myelin protein 22 (PMP22) gene. These mice present an age-dependent demyelinating neuropathy characterized by predominantly distal loss of strength and sensation. C3-PMP mice show no overt clinical signs at 3 weeks and develop mild neuromuscular impairment in an age-dependent manner. They have stable, low nerve conduction velocities similar to adults with human CMT1A. Myelination is delayed in these mice, and they contain reduced numbers of myelinated fibres at 3 weeks of age.

### Overview of the Study

Animal characteristics:
Sex/Species/Strain: B6.Cg-Tg(PMP22)C3Fbas/JMale
Age: 3 weeks old
Supplier: Jackson Laboratory (USA)
Approximate weight at initiation of treatment: 19.0 g±2.5 g
Animal number: 6 mice per group
Animal identification: At the beginning of the study, animals were identified with a number on the tail. Each parameter was noted in the lab book.
Acclimation and clinical signs: Animals arrived on site 7 days (one week) before the experiment to allow optimal acclimation. Clinical signs and mortality were recorded daily. Body weight was determined twice a week.
Study Outline:
Dose: 30 mg/kg
Administration: Oral gavage
Readouts:
i) Rotarod (neuromuscular performance)
ii) Sciatic nerve electrophysiology (EMG)
iii) PMP22 analysis by western blot
iv) Histology of sciatic nerve
Experimental time duration: Readouts measures at 1 and 2 months old.
   Baseline rotarod and EMG readouts at 1 month old
   Rotarod and EMG readouts at 2 months old
   Western blot for PMP22 and histopathology of sciatic nerve at 2 months old
Vehicle used for test Compound 163: 10% DMSO/5% Cremophor R H 40 and 85% of 10% HP-β-CD in water (v/v)
Vehicle control group: vehicle only (non-treated control)
Statistics: Statistical analyses were performed using Prism software. A probability less than 5% (p<0.05) was considered as significant.

### Operating Procedures for Study Readouts

Sciatic nerve electrophysiology. Electromyography was performed on mice anesthetized with ketamine/xylazine mixture. A pair of steel needle electrodes (AD Instruments, MLA1302) were placed subcutaneously along the nerve at the sciatic notch (proximal stimulation). A second pair of electrodes were placed along the tibial nerve above the ankle (distal stimulation). Supramaximal square-wave pulses, lasting 10 ms at 1 mA were delivered using a PowerLab 26T (AD Instruments). Compound muscle action potential (CMAP) was recorded from the intrinsic foot muscles using steel electrodes. Both amplitudes and latencies of CMAP were determined. The distance between the 2 sites of stimulation was measured alongside the skin surface with fully extended legs, and nerve conduction velocities (NCVs) were calculated automatically from sciatic nerve latency measurements using Excel.

Rotarod. A rotating rod apparatus (Bioseb, France) was used to measure neuromuscular coordination and balance. Mice were first given a pretraining trial to familiarize them with the rotating rod. Latency to fall was measured at a successively increased speed from 4 to 40 rpm over a 300-second max. time period. Each animal underwent 3 trials a day. For each day, values from the 3 trials were averaged for each animal, and then averaged for each group.

### PMP22 Western Blot Analysis

After electrophysiology analysis and animal sacrifice, the left sciatic nerve of all animals were sampled, solubilized in 4° C. RIPA lysis buffer completed with protease inhibitors (Fisher Scientific, France), sonicated three times during 10 s on ice (Microson ultrasonic cell disruptorXL, Microsonic) and centrifuged for 30 min at 10,000 rpm at 4° C. 50 μL of the supernatant was sampled and total protein was quantified by NanoDrop using bovine serum albumin dilutions as standard. Then, ten micrograms of protein samples were denatured using Laemmli/β-mercaptoethanol (Merck, S3401) incubation at 95° C. during 10 minutes, separated on a denaturing 10% SDS-polyacrylamide gel and transferred onto nitrocellulose membranes. Membranes were blocked for 60 min with 5 mL of Licor Blocking Buffer. The following primary antibodies were incubated overnight at 4° C. in the same blocking buffer: rabbit anti-PMP22 (1:750, Sigma-Aldrich, SAB4502217) and mouse anti-tubulin (1:1000, Sigma Aldrich, T9026-100UL). The following day the membranes were washed 3 times for 10 minutes in TBS Tween-20 (0.1% V/V) and then incubated for 1 hour at room temperature with the secondary fluorescence antibodies: donkey anti-mouse IRDye 680 (1:10.000, LI-COR Biosciences) and donkey anti-rabbit IRDye 800 (1:10.000, LI-COR Biosciences). After secondary antibody incubation, the membranes were washed three times for 10 min with TBS Tween-20 (0.1% V/V). Samples were leaded randomly to avoid membrane variability. Visualization of the bands was performed using Odyssey CLX LI-COR Imaging System and band quantification was performed using Image J software (version 4.0).

### Sciatic Nerve Toluidine Blue Staining

After animal sacrifice, the right sciatic nerves of 3 mice/group were fixed for 20 minutes in situ with 4% PFA and 2.5% glutaraldehyde in 0.1 M phosphate buffer (pH 7.3). Then, nerves were removed and post-fixed overnight in the same buffer. After washing for 30 minutes in 0.2 M PBS buffer, the samples were dehydrated using ethanol gradient solutions, and embedded in epoxy resin. Semithin cross sections were cut and stained with 0.5% of toluidine blue+1% borax+100 mL MilliQ water. The axonal diameter, number of myelinated axons and the myelin g-ratio were quantified using Image J g-ratio plugging.

### Summary of In Vivo Study Phase:

### Day 1: From 8 AM to 11 AM

Progressive behavioral test learning (rotarod test learning) was carried out one day before to start the in vivo part of the study. In order to reduce animal stress and anxiety due to the new environment, animals remained for 2 hours in the behavioral test room.

### Day 2: From 8 AM to 11 AM—Rotarod:

Neuromuscular performances were analyzed using a rotarod test. The values from 3 trials were averaged for each animal, and then averaged for each animal group.

### Day 3: From 8 AM to 11 AM—Electrophysiology (EMG):

The animals were anesthetized using ketamine/xylazine mixture and sciatic nerve electrophysiology recording was performed. The compound muscle action potential (CMAP) was recorded from the intrinsic foot muscles using steel electrodes. Then, the nerve conduction velocities (NCVs) of each animal were calculated from sciatic nerve latency measurements.

### From Day 3 to day 31:At 7 AM and at 7 PM

Compound 163 was administrated by oral gavage twice a day to the members of one group of mice (compound treated group). Vehicle only was administrated by oral gavage twice a day to the members of another group of mice (vehicle control group).

### Day 29: From 8 AM to 11 AM

Progressive behavioral test learning (rotarod test learning) was carried out. In order to reduce animal stress and anxiety due to the new environment, animals remained for 2 hours in the behavioral test room.

### Day 30: From 8 AM to 11 AM—Rotarod:

Neuromuscular performances were analyzed using a rotarod test. The values from 3 trials were averaged for each animal, and then averaged for each animal group

### Day 31:

### From 8 AM to 11 AM—Electrophysiology (EMG):

The animals were anesthetized using ketamine/xylazine mixture and sciatic nerve electrophysiology recording was performed. The CMAP was recorded from the intrinsic foot muscles using steel electrodes. Then, the NCVs of each animal were calculated from sciatic nerve latency measurements as stated before.

### 12 AM—Terminal Procedure, Sciatic Nerve Sampling:

After electrophysiology recording, mice were sacrificed by cervical dislocation. Left and right sciatic nerve were sampled for PMP22 western blot and histological analysis respectively.

### Results

### Mortality and Body Weight

No mortality was observed during the study.

No pathological clinical sign was observed during the study.

No significant differences on body weight were observed between groups suggesting the absence of a systemic toxic effect of Compound 163 at the analyzed time points.

Rotarod test. Similar rotarod latencies were observed in the vehicle and Compound 163 group at the baseline (1 month old). Neuromuscular impairment was observed at 2 months old in the vehicle treated group characterized by a significant decrease of the rotarod latency. In CMT1A mice at 2 months old, the Compound 163 treated group presented a significant increase of the rotarod latency compared to the vehicle group (a 133% increase in mean rotarod latency was observed in the Compound 163 treated group compared to the vehicle group). These data suggest a protective effect of Compound 163 on the neuromuscular impairment induced by the CMT1A disorder when administrated twice a day during one month at 30 mg/kg.

### Sciatic Nerve Electrophysiology.

### Compound Muscle Action Potential (CMAP) Amplitude

Similar CMAP amplitudes were observed in the vehicle and Compound 163 group at the baseline (1 month old). Sciatic nerve conduction impairment was observed at 2 months old in the vehicle treated group characterized by a significant decrease of the CMAP amplitude. In CMT1A mice at 2 months old, the Compound 163 treated group presented a significant increase of the CMAP amplitude compared to the vehicle group (an 86% increase in CMAP amplitude was observed in the Compound 163 treated group compared to the vehicle group). These data suggest a protective effect of Compound 163 compound on the CMAP impairment induced by the CMT1A disorder when administrated twice a day during one month at 30 mg/kg. Moreover, because the CMAP amplitude is directly linked to the axonal activity, these results suggest a direct or indirect efficacy of Compound 163 on the axon functionality.

### Nerve Conduction Velocity (NCV)

Concerning the nerve conduction velocities, similar nerve conduction velocities were observed in vehicle and Compound 163 groups at the baseline (1 month old). Sciatic nerve conduction impairment was observed at 2 months old in the vehicle treated group characterized by a significant decrease of the nerve conduction velocity. In CMT1A mice at 2 months old, an increase of the nerve conduction velocity was observed the Compound 163 treated group (a 94% increase in NCV was observed in the Compound 163 treated group compared to the vehicle group).

### PMP22 Western Blot Analysis

Compound 163 treated group presented a significant decrease of the overexpression of PMP22 in the sciatic nerve of CMTla mice at 2 months old compared to the vehicle group (a 33% decrease in the level of PMP22 was observed in the Compound 163 treated group compared to the vehicle group), confirming the positive efficacy of Compound 163 on the CMT1A disorder, and corroborating the behavioral test and electrophysiology data.

### Histology Analysis of Sciatic Nerve

Sciatic nerve toluidine blue staining was performed to determine the efficacy of Compound 163 from a histological point of view. In CMT1A mice at 2 months old, a significant increase of the number of myelinating axons and decrease of g-ratio was observed after Compound 163 treatment compared to the vehicle group (a 64% increase in the number of myelinating axons and a 22% decrease in the g-ratio). In CMT1A mice at 2 months old, an increase in the axonal diameter was also observed in the Compound 163 treatment group compared to the vehicle group (a 45% increase). These data confirm the functional efficacy data observed using the behavioral test and electrophysiological analysis and suggest that Compound 163 targets axonal and myeling Schwann cell pathology induced by CMT disorder.

### SUMMARY AND CONCLUSIONS

Neuromuscular impairment and decrease of nerve conduction amplitude and velocity were observed in the preclinical CMT1A mouse model treated with vehicle. The Compound 163 treated group presented a significant increase of the rotarod latency and CMAP amplitude, decrease of PMP22 overexpression and an increase in the number of axons and myelin diameter compared to the vehicle group. Also, an increase of the nerve conduction velocity and axonal diameter were also observed in the Compound 163 treated group but these increases were not statistically significant compared to the vehicle group.

Taken together, this study confirms that Compound 163 presents significant protective effect on CMT1A neuropathy targeting myelin sheath and axons. The compound increases neuromuscular and electrophysiological performances and decreases the molecular pathomechanism when administrated twice a day during 30 days at 30 mg/kg.

This model functions as an in vivo model of Charcot-Marie-Tooth 1A (CMT1A). Activity (positive effects) in this model strongly supports that compounds of this invention are efficacious in the treatment of Charcot-Marie-Tooth 1A (CMT1A and other autophagy-related diseases or conditions.

The invention claimed is:

1. A pharmaceutical composition comprising one or more of a compound according to Formula (I), 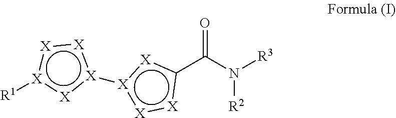
a physiologically acceptable salt of the compound according to Formula (I), a solvate of the compound according to Formula (I), a hydrate of the compound according to Formula (I), an enantiomer of the compound of Formula (I), and a polymorph of the compound according to Formula (I),
wherein
X is independently selected from chemically possible combinations of C, N, O, and S, and is optionally substituted with one of —CH₃, —CH₂—CH₃ or COOH;
R¹ is selected from cyclic C₅ or C₆ alkyl, optionally comprising chemically possible N, O, and S, and optionally mono- or bi-substituted with one or more of —CH₃, —NH₂, —COOH, C₁ to C₄ alkoxy, halo, trifluoromethyl, and trifluoromethoxy;
R² is selected from H, CH₃, straight or branched C₂ to C₆ alkyl, optionally comprising at least one of chemically possible N, O, S, and optionally substituted with one of —CH₃, —NH₂, —OH, —COOH, cyclopentyl, cyclohexyl, bicyclo[2.2.1]heptane, and bicyclo[3.1.1]heptane, bicyclo[2.2.2]octane, optionally comprising chemically possible N, O, and S, and optionally substituted with one of —CH₃, —CH₂—CH₃, —OH, -isopropyl, —COOH, —COOCH₃, —CH₂-cyclohexyl, and NH₂;
R³ is selected from H, straight or branched C₁ to C₆ alkyl, optionally substituted with one of —CH₃, —OH, —CH₂—CH₃, —NH₂, —CH₂—NH₂, —CH₂—NH—CH₃, —COOH, optionally comprising chemically possible one or more N, O, and S;
the C₁ to C₄ alkoxy, optionally at least one of chemically possible one or more N, O, and S, and optionally mono- or bi substituted with, —NH₂, —OH, —COOH;
cyclopropyl, cyclobutyl, optionally comprising chemically possible at least one of one or more N, O, S, and optionally mono- or bi substituted with one or more of —CH₃, —CH₂—CH₃, —NH₂, —CH₂—NH₂, —CH₂—NH—CH₃, —OH, —COOH;
with the provisio that R² and R³ are not both H or CH₃;
R2 and R3 may be connected to form a 4-membered, 5-membered, 6-membered, 7-membered ring, a bicyclic ring structure of at least one of fused cyclopentyl and cyclohexyl, bicyclo[3.1.1]heptane, optionally comprising chemically possible one or more N, O, and S, and optionally being mono- or bi-substituted with one or more of straight and branched C1 to C6 alkyl, optionally substituted with one or more of —CH₃, —OH, —CH₂—CH₃, —NH₂, —NH—CH₃, —CH₂—NH₂, —CH₂—NH—CH₃,—OH, —COOH, —COOCH₃, ═O, isopropyl, sulfonamide, and optionally including chemically possible one or more N, O, and S;
with the provisio that R², R³, or the combination thereof comprises at least one NH or NH₂.

2. A pharmaceutical composition according to claim 1, wherein the compound according to Formula (I) is also according to the Formula (II), 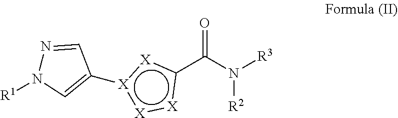
wherein
X, R¹, R² and R³ are as above.

3. A compound according to claim 1, wherein the compound according to Formula (I) is also according to Formula III), 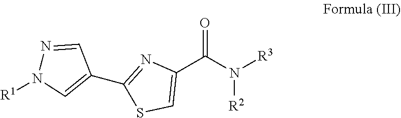
wherein
R¹, R² and R³ are as above.

4. A pharmaceutical composition according to claim 1, wherein
R¹ is selected from functional groups 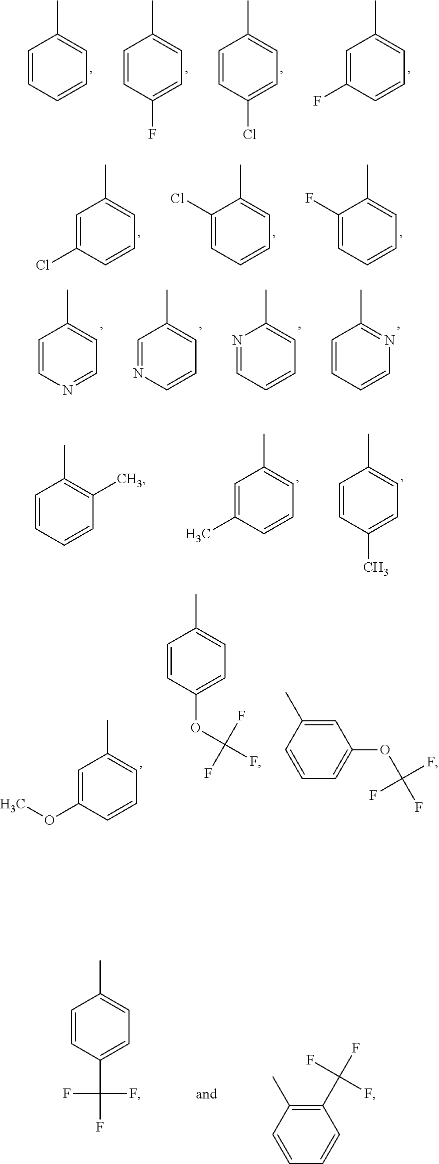
a physiologically acceptable salts of the groups, solvates of the groups, hydrates of the groups, enantiomers of the groups, and polymorphs of the groups.

5. A pharmaceutical composition according to any one of claim 1, wherein
R² is selected from functional groups H, —CH₃, —CH₂—CH₃, —CH₂—CH₂—CH₃, —CH₂—CH₂—NH₂, —CH₂—CH₂—CH₂—CH₂—NH₂, —CH₂—CH₂—CH₂—CH₂—CH₂—NH₂, —CH₂—CH₂—O—CH₂—CH₂—NH₂, cyclohexyl, —CH₂(CH₃)—CH₂—NH₂ 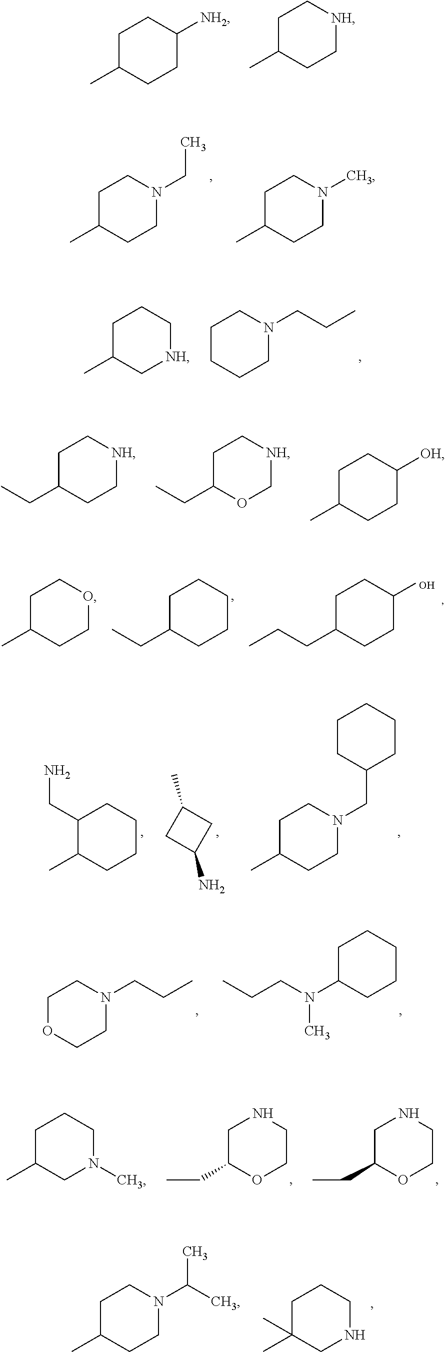 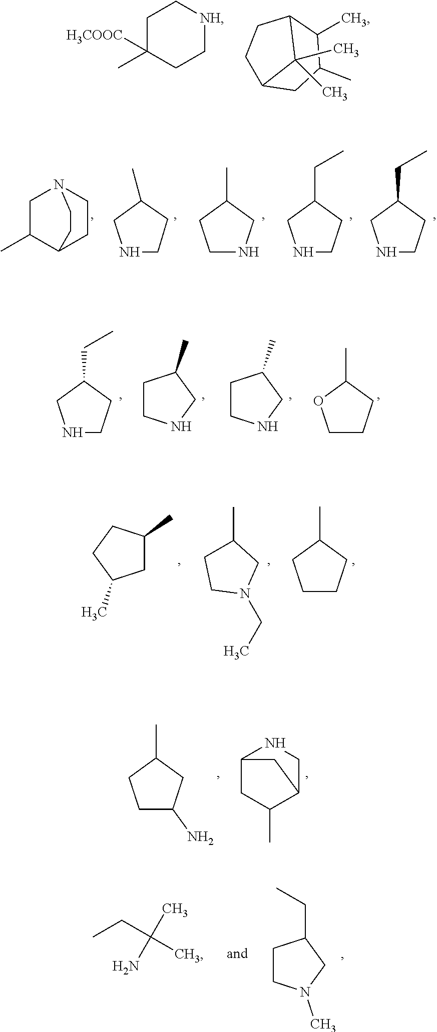
R³ is selected from further functional groups H, —CH₃, —CH₂—CH₃, propyl, isopropyl, —CH₂—CH₂—OH, —CH₂—CH₂—NH₂, —CH₂—CH₂—CH₂—NH₂, —CH(NH₂)—CH₃, —CH₂—CH₂—CH₂—CH₂—CH₂—NH₂, —CH₂—CH₂—O—CH₂—CH₂—NH₂, —CH₂—CH(CH₃)₂, —CH(CH₃)₂, —CH₂—CH(NH₂)—CH₃, —CH(—CH₃)—CH₂—NH₂, —CH₂—CH₂—N(CH₃)₂, 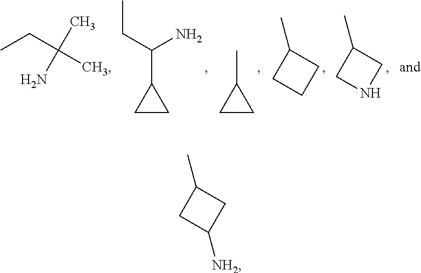
and
R² and R³ may be connected to form a ring selected from still further functional groups 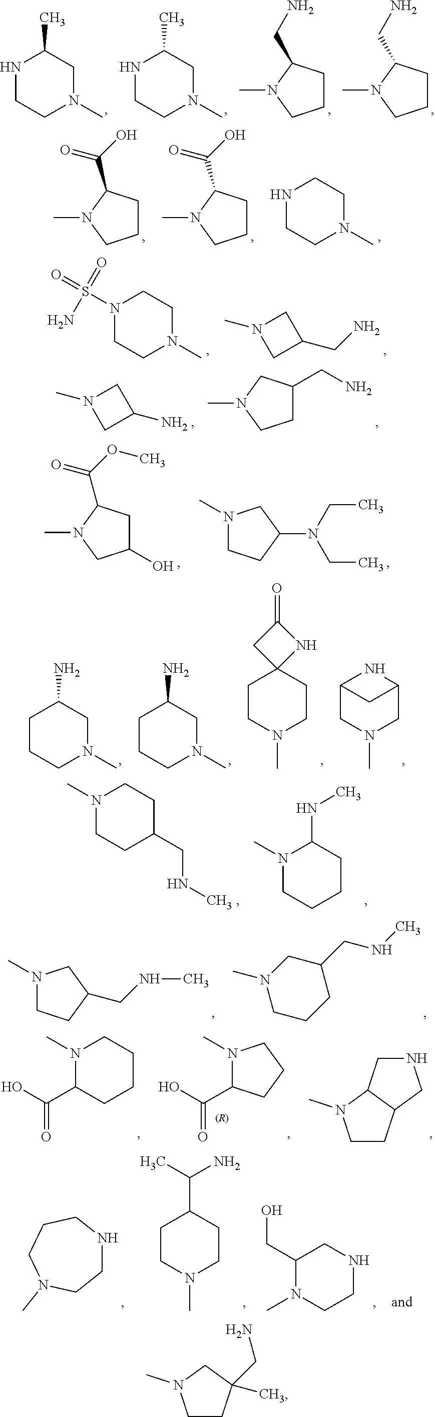
physiologically acceptable salts of the still further functional groups, solvates of the still further functional groups, hydrates of the functional groups, enantiomers of the still further functional groups, and polymorphs of the still further functional groups.

6. A pharmaceutical composition according to claim 1, wherein the compound according to Formula (I) is also according to the following, Formula (IV), 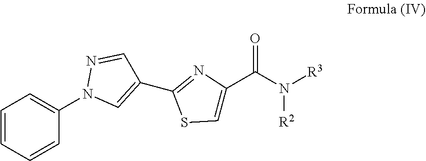
wherein R² is selected from functional groups 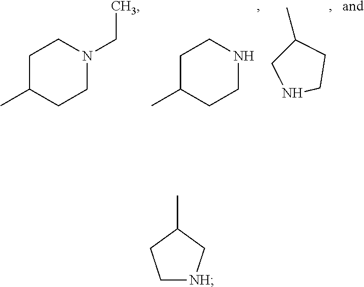
one of R³ is selected from further functional groups H, CH₃, —CH₂—CH₂—CH₃, and isopropyl, and
R² and R³ form a ring selected from still further functional groups 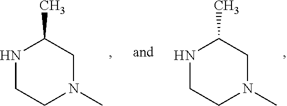
physiologically acceptable salts of the still further functional groups, solvates of the still further functional groups, hydrates of the still further functional groups, enantiomers of the still further functional groups, and polymorphs of the still further functional groups.

7. A pharmaceutical composition comprising one or more of a physiologically acceptable salt of a compound, a solvate of the compound, a hydrate of the compound, an enantiomer of the compound, and a polymorph of the compound, wherein the compound is selected from the following:
5-(1-phenyl-1H-pyrazol-4-yl)-N-propyl-N-[(3S)-pyrrolidin-3-yl]-1H-pyrrole-2-carboxamide (Formula 93);
5-(1-phenyl-1H-pyrazol-4-yl)-N-propyl-N-[(3R)-pyrrolidin-3-yl]-1H-pyrrole-2-carboxamide (Formula 94);
2-(5-phenylthiophen-2-yl)-N-propyl-N-(pyrrolidin-3-yl)-1,3-thiazole-4-carboxamide (Formula 122);
4-(1-phenyl-1H-pyrazol-4-yl)-N-propyl-N-(pyrrolidin-3-yl)-1H-pyrrole-2-carboxamide (Formula 123);
2-(1-phenyl-1H-pyrazol-4-yl)-N-(piperidin-4-yl)-N-(propan-2-yl)-1,3-thiazole-4-carboxamide (Formula 163);
2-(1-phenyl-1H-pyrazol-4-yl)-N-propyl-N-[(3S)-pyrrolidin-3-yl]-1,3-thiazole-4-carboxamide (Formula 170);
2-(1-phenyl-1H-pyrazol-4-yl)-N-propyl-N-[(3R)-pyrrolidin-3-yl]-1,3-thiazole-4-carboxamide (Formula 171).

8. A pharmaceutical composition according to claim 1 and exhibiting higher systemic (peripheral) concentrations compared to the CNS or at a minimum of a 2:1 peripheral/brain ratio, after an administration thereof.

9. (canceled)

10. A pharmaceutical composition according to claim 1, further comprising a pharmaceutically acceptable carrier.

11. A pharmaceutical composition according to claim 1, wherein the compound is for use in at least one of the prevention and treatment of diseases in a mammalian subject.

12. A pharmaceutical composition according to claim 1, wherein the compound is for use as an autophagy inducer, wherein preferably the use is at least one of cosmetic and in vitro.

13. A pharmaceutical composition according to claim 1, wherein the compound is for use in prevention and treatment of an autophagy-related disease or condition in a mammalian subject.

14. A pharmaceutical composition for use according to claim 13, wherein said autophagy-related disease or condition is selected from the group consisting of systemic lupus erythematosus, cancer, liver diseases, al antitrypsin deficiency, Charcot Marie Tooth syndrome, Rett Syndrome, Sickle Cell disease, Wilson Disease, amyloidosis, Gaucher's diseases, lysosomal and glycogen storage disorders, cystic fibrosis; viral infection and diseases, human cytomegalovirus (HCMV) infection, hepatitis B, human immunodeficiency virus infection, Zika virus infection, coronavirus infection, HCoV-229E, HCoV-NL63, betacoronavirus infection, such as HCoV—OC43, SARS-CoV-1, HCoV—HKU1, MERS-CoV or SARS-CoV-2, bacterial infections, metabolic disorders, diabetes, fibrosis, wound healing disorders, Niemann-Pick type C (NPC) disease, fibrinogen storage disease (FSB), inclusion body disease (IBD), muscular dystrophy, Duchenne muscular dystrophy, Limb-girdle muscular dystrophy, myopathy, myofibrillar myopathy, hereditary myopathy, diabetic cardiomyopathy, anti-inflammatory disorders, autoimmune diseases, multiple sclerosis, rheumatoid arthritis, irritable bowel syndrome, Crohn's disease, vascular disorders, coronary artery diseases, myocardial infarction, unstable angina pectoris, atherosclerosis or vasculitis, Behcet's syndrome, giant cell arteritis, polymyalgia rheumatica, Wegener's granulomatosis, Churg-Strauss syndrome, vasculitis, Henoch-Schonlein purpura, Kawasaki disease, viral infection or replication, pox virus infection, herpes virus infection, asthma, allergic rhinitis, COPD, osteoporosis, organ transplant rejection, psoriasis, hypertrophic scarring (keloid formation), adhesion formations following general or gynecological surgery, lung fibrosis, liver fibrosis, kidney fibrosis, disorders caused by intracellular parasites, malaria, tuberculosis, neuropathic pain, post-operative phantom limb pain or postherpetic neuralgia, allergies, antigen induced recall response, immune response suppression, muscle degeneration and atrophy, frailty in aging, spinal cord injury, and diseases and conditions involving at least one of misfolded and nonfolded proteins.

15. A pharmaceutical composition according to claim 1, further comprising at least one additional pharmaceutically active substance for the autophagy-related disease or condition.

16. A pharmaceutical composition for use according to claim 13, wherein said prevention and treatment comprises a systemic treatment, exhibiting higher systemic (peripheral) concentrations compared to the central nervous system or brain.

17. A pharmaceutical composition for use according to claim 13, wherein said prevention and treatment further comprise at least one detecting and monitoring in said subject a response of at least one autophagy-biomarker, preferably selected from the group of BECN1 and ATG8/LC3 family comprising LC3A, LC3B, LC3C), LC3-II, ULK1, p62, NBR1, ATG5 and ATG7.

18. A method for preventing and treating an autophagy-related disease or condition in a mammalian subject comprising administering to said mammalian subject an effective amount of a pharmaceutical composition according to claim 1.

19. A method according to claim 18, wherein said autophagy-related disease or condition is selected from the group consisting of systemic lupus erythematosus, cancer, liver diseases, al antitrypsin deficiency, Charcot Marie Tooth syndrome, Rett Syndrome, Sickle Cell disease, Wilson Disease, amyloidosis, Gaucher's diseases, lysosomal and glycogen storage disorders, cystic fibrosis; viral infection and diseases, human cytomegalovirus (HCMV) infection, hepatitis B, human immunodeficiency virus infection, Zika virus infection, coronavirus infection, HCoV-229E, HCoV-NL63, betacoronavirus infection, such as HCoV—OC43, SARS-CoV-1, HCoV—HKU1, MERS-CoV or SARS-CoV-2, bacterial infections, metabolic disorders, diabetes, fibrosis, wound healing disorders, Niemann-Pick type C (NPC) disease, fibrinogen storage disease (FSB), inclusion body disease (IBD), muscular dystrophy, Duchenne muscular dystrophy, Limb-girdle muscular dystrophy, myopathy, myofibrillar myopathy, hereditary myopathy, diabetic cardiomyopathy, anti-inflammatory disorders, autoimmune diseases, multiple sclerosis, rheumatoid arthritis, irritable bowel syndrome, Crohn's disease, vascular disorders, coronary artery diseases, myocardial infarction, unstable angina pectoris, atherosclerosis or vasculitis, Behcet's syndrome, giant cell arteritis, polymyalgia rheumatica, Wegener's granulomatosis, Churg-Strauss syndrome, vasculitis, Henoch-Schonlein purpura, Kawasaki disease, viral infection or replication, pox virus infection, herpes virus infection, asthma, allergic rhinitis, COPD, osteoporosis, organ transplant rejection, psoriasis, hypertrophic scarring (keloid formation), adhesion formations following general or gynecological surgery, lung fibrosis, liver fibrosis, kidney fibrosis, disorders caused by intracellular parasites, malaria, tuberculosis, neuropathic pain, post-operative phantom limb pain or postherpetic neuralgia, allergies, antigen induced recall response, immune response suppression, muscle degeneration and atrophy, frailty in aging, spinal cord injury, and diseases and conditions involving misfolded and/or nonfolded proteins.

20. A method according to claim 18, wherein said prevention and treatment comprise a systemic treatment, exhibiting higher systemic (peripheral) concentrations of the compound compared to the central nervous system or brain.

21. A method according 18, further comprising at least one of detecting and monitoring in said subject a response of at least one autophagy-biomarker, selected from the group of BECN1 and ATG8/LC3 family comprising LC3A, LC3B, LC3C), LC3-II, ULK1, p62, NBR1, ATG5 and ATG7.


SEQUENCE LISTING (2 sequences)

SEQ ID NO: 1 - DNA, 21 bp, Homo sapiens
  source 1..21: mol_type = genomic DNA
catcagccctaatccatctga

SEQ ID NO: 2 - DNA, 21 bp, Homo sapiens
  source 1..21: mol_type = genomic DNA
cgcgactaacaatcaaagtga